(19) United States
(12) Patent Application Publication
(10) Pub. No.: US 2006/0079787 A1
(43) Pub. Date: Apr. 13, 2006

(54) Transmembrane access systems and methods

(76) Inventors: James S. Whiting, Los Angeles, CA (US); Neal L. Eigler, Pacific Palisades, CA (US); John L. Wardle, San Clemente, CA (US); Werner Hafelfinger, Thousand Oaks, CA (US); Brian Mann, Edgartown, MA (US)

Correspondence Address: LAW OFFICE OF WILLIAM B. ANDERSON, 619 SOUTH VULCAN AVENUE, SUITE 214B, ENCINITAS, CA, 92024, US

(21) Appl. No.: 11/203,624

(22) Filed: Aug. 11, 2005

Related U.S. Application Data
(63) Continuation-in-part of application No. 10/956,899, filed on Sep. 30, 2004.

(51) Int. Cl. A61B 8/14

(52) U.S. Cl. 600/466

(57) ABSTRACT

Systems and methods for penetrating a tissue membrane to gain access to a target site are disclosed. In some examples, systems and methods for accessing the left atrium from the right atrium of a patient's heart are carried out by puncturing the intra-atrial septal wall. One embodiment provides a system for transseptal cardiac access that includes a stabilizer sheath having a side port, a shaped guiding catheter configured to exit the side port and a tissue penetration member disposed within and extendable from the distal end of the guide catheter. The tissue penetration member may be configured to penetrate tissue upon rotation and may be coupled to a distal portion of a torquable shaft. In some embodiments, the stabilizer sheath and shaped guiding catheter may be moved relative to the patient's body structure and relative to each other so that a desired approach angle may be obtained for the tissue penetration member with respect to the target tissue.

### RELATED APPLICATIONS

This application is a continuation-in-part of U.S. patent application Ser. No. 10/956,899, filed Sep. 30, 2004 by Whiting et al., titled Transmembrane Access Systems and Methods, which is incorporated by reference herein in its entirety.

### BACKGROUND

Access to the left side of the heart plays an important role in the diagnosis and treatment of cardiovascular disease. Invasive cardiologists commonly perform a left heart catheterization for angiographic evaluation or transcatheter intervention of cardiac or coronary artery disease. In a left heart catheterization, the operator achieves vascular access through a femoral artery and passes a catheter in a retrograde direction until the catheter tip reaches the coronary artery ostia or crosses the aortic valve and into the left ventricle. From a catheter positioned in the left ventricle, an operator can measure left ventricular systolic and end-diastolic pressures and evaluate aortic valve disease. Ventriculography, where contrast is injected into the left ventricle, may be performed to evaluate left ventricular function. Alternative insertion sites, such as the brachial or radial artery, are used sometimes when femoral artery access is contraindicated due to iliofemoral atherosclerosis, but manipulation of the catheter can be more difficult from these other insertion sites.

Although left heart catheterization can be a fast and relatively safe procedure for access to the coronary arteries and the left ventricle, its usefulness for accessing structures beyond the left ventricle, namely the left atrium and the pulmonary veins, is limited by the tortuous path required to access these structures from the left ventricle via the mitral valve. For example, electrophysiologic procedures requiring access to the left atrium or pulmonary veins, performance of balloon mitral valve commissurotomy, and left ventricular access across an aortic prosthetic disc valve can be difficult, and sometimes unfeasible, through traditional left heart catheterization techniques.

Transseptal cardiac catheterization is another commonly employed percutaneous procedure for gaining access to the left side of the heart from the right side of the heart. Access occurs by transiting across the fibro-muscular tissue of the intra-atrial septum from the right atrium and into the left atrium. From the left atrium, other adjoining structures may also be accessed, including the left atrial appendage, the mitral valve, left ventricle and the pulmonary veins.

Transseptal cardiac catheterization has been performed in tens of thousands of patients around the world, and is used for both diagnostic and therapeutic purposes. Diagnostically, operators utilize transseptal catheterization to carry out electrophysiologic procedures requiring access to the pulmonary veins and also to do left heart catheterizations where a diseased aortic valve or an aortic disc prosthetic valve prohibits retrograde left ventricular catheterization across the valve. Therapeutically, operators employ transseptal cardiac catheterization to perform a host of therapeutic procedures, including balloon dilatation for mitral or aortic valvuloplasty and radiofrequency ablation of arrhythmias originating from the left side of the heart. Transseptal cardiac catheterization is also used to implant newer medical devices, including occlusion devices in the left atrial appendage for stroke prevention and heart monitoring devices for the treatment of cardiovascular disease.

The vast majority of transseptal procedures is performed via a femoral vein access site, using special set of devices, called a Brockenbrough needle and catheter/dilator, designed for this approach. In this standard approach the Brockenbrough catheter/dilator, with the hollow Brockenbrough needle within, is advanced from a femoral vein, through the inferior vena cava, through the right atrium and into the superior vena cava. The distal end is then pulled back to the right atrium and rotated until it points at the foramen ovale of the atrial septum. The Brockenbrough needle has a gentle bend that facilitates guiding the system from the vena cava into and through the right atrium, to the intra-atrial septum. The right atrial surface of the septum faces slightly downward, toward the inferior vena cava, so that the natural path of the Brockenbrough needle/catheter brings it to the atrial surface at nearly a right angle of incidence. After verifying the location of the catheter tip at the septal surface by fluoroscopy and/or ultrasound imaging, the operator can firmly but gradually advance the needle within the catheter until its tip penetrates the septum. Contrast material is then injected through the lumen of the Brockenbrough needle and observed fluoroscopically to verify placement of the tip in the left atrium. Once this placement is verified, the catheter/dilator may be advanced through the septum into the left atrium, the Brockenbrough needle is removed and a guide wire can be placed into the left atrium through the dilator lumen. At this point, access to the left atrium has been established and the Brockenbrough needle can be removed, allowing introduction of other devices either over the guide wire or through a Mullins sheath placed over the dilator, or both, as is well known to those skilled in the art.

Transseptal cardiac catheterization using the standard technique described above is generally successful and safe when performed by skilled individuals such as invasive cardiologists, interventional cardiologists, and electrophysiologists with appropriate training and experience. Lack of success may be attributable to anatomic variations, especially with respect to the size, location and orientation of the pertinent cardiovascular structures and imaging-related anatomic landmarks. Another reason for failure may be the relatively fixed dimensions and curvatures of currently available transseptal catheterization equipment. One major risk of existing transseptal catheterization techniques lies in the inadvertent puncture of atrial structures, such as the atrial free wall or the coronary sinus, or entry into the aortic root or pulmonary artery. In some cases, these punctures or perforations can lead to bleeding around the heart resulting in impaired cardiac function known as cardiac tamponade, which if not promptly recognized and treated, may be fatal. As such, surgical repair of such a cardiac perforation is sometimes required.

One problem with the standard transseptal needle/catheter system is that once an inadvertent puncture has occurred, it may be difficult to realize what structure has been compromised because contrast injection through the needle is limited by the small bore lumen thereof. Thus, visualization of the structure entered may be inadequate and non-diagnostic. Also, the tip of the catheter dilator of existing devices may cross the puncture site which has the effect of further enlarging the puncture hole.

Other than minor refinements in technique and equipment, the standard transseptal catheterization procedure has remained relatively constant for years. Even so, the technique has several recognized limitations that diminish the efficacy and safety of this well-established procedure. Thus, there remains a need for an alternative system that effectively and safely provides access to the left atrium, or other desired site in the body.

As noted above, standard transseptal cardiac catheterization is performed via the inferior vena cava approach from an access site in a femoral vein. In some situations it is clinically desirable to perform transseptal cardiac catheterization via the superior vena cava from an access site in a vein in the neck or shoulder area, such as a jugular or subclavian vein. The superior vena cava approach is more problematic than the standard inferior vena cava approach because of the downward anatomical orientation of the intra-atrial septum, mentioned above. For such an approach the Brockenbrough needle must make more than a 90° bend to engage the atrial septum at a right angle of incidence, which makes it difficult to exert a sufficient force along the axis of the needle to penetrate the septum. In fact, it is in general problematic to exert an axial force around a bend in a flexible wire, rod, needle, or other elongated member, because the axial force tends to bend or flex the device rather than simply translate it axially. Thus, there is a need for improved apparatus and methods for performing procedures requiring an axial force, such as punctures, when a bend in the flexible member transmitting the force is unavoidable. Another problem not infrequently encountered with conventional transseptal catheterization is that advancement of a Brockenbrough needle against the septum can cause substantial displacement or tenting of the septum from right to left prior to puncture. Sudden penetration can result in the needle injuring other structures in the left atrium. As such, what has been needed are systems and methods that provide for the reduction or elimination of the force required to perform the procedure, such as a transseptal puncture; and provision of a stabilizing apparatus for transmitting an axial force around a bend.

### SUMMARY

One embodiment is directed to a transmembrane access system having a stabilizer sheath with a tubular configuration and an inner lumen extending therein and having a side port disposed on a distal section of the sheath and in communication with the inner lumen. The system also includes a tubular guide catheter having a shaped distal section that has a curved configuration in a relaxed state and an outer surface which is configured to move axially within a portion of the inner lumen of the stabilizer sheath that extends from the proximal end of the stabilizer sheath to the side port. A tissue penetration member is disposed within a distal end of the guiding catheter and is axially extendable from the distal end of the guiding catheter for membrane penetration. In one particular embodiment, the tissue penetration member is configured to penetrate tissue upon rotation and the system further includes an elongate torquable shaft coupled to the tissue penetration member.

Another embodiment of a transmembrane access system includes a tubular guide catheter having a shaped distal section that has a curved configuration in a relaxed state. A tissue penetration member configured to penetrate tissue on rotation includes a helical tissue penetration member. The tissue penetration member is configured to move axially within an inner lumen of the tubular guide catheter and is axially extendable from the guide catheter for membrane penetration. An activation modulator is coupled to the tissue penetration member by a torquable shaft and is configured to axially advance and rotate the torquable shaft upon activation of the activation modulator.

One embodiment of a method of use of a transmembrane access system includes a method of accessing the left atrium of a patient's heart from the right atrium of the patient's heart wherein a transmembrane access system is provided. The transmembrane access system includes a stabilizer sheath having a tubular configuration with an inner lumen extending therein and a side port disposed on a distal section of the sheath in communication with the inner lumen. The system also includes a tubular guide catheter having a shaped distal section that has a curved configuration in a relaxed state and an outer surface which is configured to move axially within a portion of the inner lumen of the stabilizer sheath that extends from the proximal end of the stabilizer sheath to the side port. A tissue penetration member is disposed within a distal end of the guiding catheter and is axially extendable from the distal end of the guiding catheter for membrane penetration.

Once the transmembrane access system has been provided, the stabilizer sheath is advanced over a guidewire from the vascular access site in a subclavian or jugular vein through superior vena cava of the patient and positioned with the distal end of the stabilizer sheath within the inferior vena cava with the side port of the stabilizer sheath within the right atrium facing the intra-atrial septum of the patient's heart. The guidewire is removed and the distal end of the guide catheter is advanced through the inner lumen of the stabilizer sheath until the distal end of the guide catheter exits the side port of the stabilizer sheath and is positioned adjacent target tissue of a desired site of the septum of the patient's heart. The tissue penetration member is advanced from the distal end of the guide catheter and activated so as to penetrate the target tissue. For some embodiments, the tissue penetration member is activated by rotation of the tissue penetration member. The tissue penetration member is then advanced distally through the septum.

Another embodiment of using a transmembrane access system includes a method of accessing a second side of a tissue membrane from a first side of a tissue membrane wherein a transmembrane access system is provided. The transmembrane access system includes a guide catheter with a shaped distal section that has a curved configuration in a relaxed state. The system also includes a tissue penetration member which is disposed within a distal end of the guide catheter and which is axially extendable from the distal end of the guide catheter for membrane penetration. The tissue penetration member is configured to penetrate tissue upon rotation and has a guidewire lumen disposed therein. The distal end of the guide catheter is positioned until the distal end of the guide catheter is adjacent to a desired site on the first side of the tissue membrane.

The tissue penetration member is advanced distally from the guide catheter until the distal end of the tissue penetration member is in contact with the tissue membrane. The tissue penetration member is then rotated and advanced distally through the tissue membrane. Contrast material may be injected through the guidewire lumen of the penetrating member while observing fluoroscopically to verify that the tissue penetration member has entered the desired distal chamber. Also, pressure can be monitored through the guidewire lumen to verify that the tissue penetration member has entered the desired distal chamber. Contrast may be injected under fluoroscopic observation as well as monitoring of pressure through the same lumen to verify positioning of the tissue penetration member. Finally, a guidewire is advanced through the guidewire lumen of the tissue penetration member until a distal end of the guidewire is disposed on the second side of the tissue membrane.

In another embodiment, a transmembrane access system includes a stabilizer sheath having an inner lumen extending therein and having a side port disposed on a distal section of the stabilizer sheath and in communication with the inner lumen. A guide catheter having a shaped distal section that has a curved configuration in a relaxed state and an outer surface is configured to move axially within a portion of the inner lumen of the stabilizer sheath that extends from the proximal end of the stabilizer sheath to the side port. A tissue penetration member is configured to move axially within an inner lumen of the guide catheter and is axially extendable from the guide catheter for membrane penetration. An ultrasound emission element and an ultrasound receiver are disposed at the distal section of the stabilizer sheath.

In another embodiment, a transmembrane access system includes a guide catheter having a shaped distal section that has a curved configuration in a relaxed state. A tissue penetration member which is axially extendable from the guide catheter is provided for membrane penetration, and an ultrasound emission member and an ultrasound receiver are disposed adjacent the shaped distal section of the guide catheter.

In another embodiment of a method of accessing the left atrium of a patient's heart from the right atrium of the patient's heart, a transmembrane access system is provided. The transmembrane access system includes a stabilizer sheath having an inner lumen extending therein and having a side port disposed on a distal section of the sheath and in communication with the inner lumen. The transmembrane access system also includes a guide catheter having a shaped distal section that has a curved configuration in a relaxed state and an outer surface which is configured to move axially within a portion of the inner lumen of the stabilizer sheath that extends from the proximal end of the stabilizer sheath to the side port. A tissue penetration member is also included which is configured to move axially within an inner lumen of the tubular guide catheter and which is axially extendable from the distal end of the guide catheter for membrane penetration. Finally, the access system includes an ultrasound emission element and an ultrasound receiver disposed at the distal section of the stabilizer sheath.

Once the transmembrane access system has been provided, the stabilizer sheath is advanced through a superior vena cava of the patient and positioned with the distal end of the sheath within the inferior vena cava and with the side port of the stabilizer sheath facing the right atrium of the patient's heart. The distal end of the guide catheter is advanced through the inner lumen and out the side port of the stabilizer sheath. Ultrasound energy is then emitted from the ultrasound emission member directed towards a desired site of tissue penetration. Reflected ultrasound energy is then received with the ultrasound receiver and information is generated from the reflected ultrasound energy about the desired site. In some embodiments, the information may include the location of the guide catheter relative to the atrial septum or other body structures. On some embodiments, the position of the distal end of the guide catheter is adjusted by advancing or withdrawing the guide catheter within the stabilizer sheath, advancing or withdrawing the stabilizer sheath, twisting the guide catheter to the right or the left, twisting the stabilizer sheath to the right or the left, or a combination of any of these maneuvers, until the distal end of the guide catheter is positioned adjacent a desired site of the septum of the patient's heart. This positioning may be facilitated by the information generated from the reflected ultrasound energy. The tissue penetration member is advanced from the distal end of the guide catheter, actuated, and advanced distally through the septum.

In an embodiment of a method of accessing a second side of a tissue membrane from a first side of a tissue membrane, a transmembrane access system is provided that includes a guide catheter with a shaped distal section that has a curved configuration in a relaxed state. The system also includes a tissue penetration member which is disposed within a distal end of the guide catheter and which is axially extendable from the distal end of the guide catheter for membrane penetration. An ultrasound emission member and an ultrasound receiver are disposed at a distal portion of the guide catheter. The distal end of the guide catheter is positioned until the distal end of the guide catheter is near a desired site on the first side of the tissue membrane. The ultrasound emission member emits ultrasound energy directed towards the desired site. Reflected ultrasound energy is then received with the ultrasound receiver and information is generated from the reflected ultrasound energy about the desired site. For some embodiments, such information may include the location of the guide catheter relative to the atrial septum or other body structures. On some embodiments, the position of the distal end of the guide catheter is adjusted by advancing or withdrawing the guide catheter within the stabilizer sheath, advancing or withdrawing the stabilizer sheath, twisting the guide catheter to the right or the left, twisting the stabilizer sheath to the right or the left, or a combination of any of these maneuvers, until the distal end of the guide catheter is positioned adjacent a desired site of the septum of the patient's heart. Such positioning may be facilitated by the information generated from the reflected ultrasound energy. The tissue penetration member is advanced distally from the guide catheter until the distal end of the tissue penetration member is adjacent the tissue membrane at the desired site. The tissue penetration member is activated so as to penetrate distally through the tissue membrane and thereafter a guidewire may then be advanced through a guidewire lumen of the tissue penetration member until a distal end of the guidewire is disposed on the second side of the tissue membrane.

In an embodiment of a method of positioning an access catheter within a chamber of a patient's body, an access system is provided including a stabilizer sheath having a tubular configuration with an inner lumen extending therein and having a side port disposed on a distal section of the sheath and in communication with the inner lumen, a guide catheter having a shaped distal section that has a curved configuration in a relaxed state and an outer surface which is configured to move axially within a portion of the inner lumen of the stabilizer sheath that extends from the proximal end of the stabilizer sheath to the side port, and an ultrasound emission member and an ultrasound receiver disposed at the distal section of the stabilizer sheath. The stabilizer sheath is advanced through a first tubular structure of the patient which is in fluid communication with the chamber. The stabilizer sheath is further positioned with the side port of the stabilizer sheath adjacent to the chamber of the patient's body and with a portion of the stabilizer sheath distal of the side port into a second tubular structure which is also in fluid communication with the chamber. The distal end of the guide catheter is advanced through the inner lumen of the stabilizer sheath until the distal end of the guide catheter exits the side port of the stabilizer sheath. Ultrasound energy is emitted by the ultrasound emission member directed towards a desired site within the chamber. Reflected ultrasound energy is received with the ultrasound receiver and information about the desired site is generated from the reflected ultrasound energy. The distal end of the guide catheter is then positioned adjacent the desired site of the chamber. In some embodiments, the stabilizer sheath and/or the guide catheter is rotated and axially translated until the distal end of the guide catheter is positioned adjacent the desired site of the chamber. Such positioning may be facilitated in some embodiments by the information about the desired site generated from the reflected ultrasound energy.

In another embodiment, a transmembrane access system includes a stabilizer sheath having an inner lumen extending therein, having a side port disposed on a distal section of the sheath and in communication with the inner lumen and having a curled section on a distal portion of the distal section wherein the discharge axis of the distal end of the elongate tubular shaft is greater than 180 degrees from the longitudinal axis of the stabilizer sheath proximal of the curled section and wherein the curled section is directed opposite the side port with respect to circumferential orientation about the stabilizer sheath. The access system also includes a guide catheter having a shaped distal section that has a curved configuration in a relaxed state and an outer surface which is configured to move axially within a portion of the inner lumen of the stabilizer sheath that extends from the proximal end of the stabilizer sheath to the side port. A tissue penetration member is configured to move axially within an inner lumen of the guide catheter and is axially extendable from a distal end of the guide catheter for membrane penetration.

In another embodiment, a transmembrane access system includes a stabilizer sheath having an inner work lumen extending therein, a port disposed on a distal section of the sheath and in communication with the inner lumen and a stabilizer member lumen substantially parallel to a longitudinal axis of the stabilizer sheath disposed at the distal section of the stabilizer sheath. An elongate stabilizer member is configured to extend from the stabilizer member lumen and provide lateral support to the distal end of the stabilizer sheath. A guide catheter having a shaped distal section with a curved configuration in a relaxed state has an outer surface which is configured to move axially within a portion of the inner lumen of the stabilizer sheath that extends from the proximal end of the stabilizer sheath to the port. A tissue penetration member is configured to move axially within an inner lumen of the tubular guide catheter and is axially extendable from the guide catheter for membrane penetration.

In another embodiment of a method of accessing the left atrium of a patient's heart from the right atrium of the patient's heart, a transmembrane access system is provided having a stabilizer sheath with an inner work lumen extending therein, a port disposed on a distal end of the sheath and in communication with the inner lumen and having a stabilizer member lumen substantially parallel to a longitudinal axis of the stabilizer sheath disposed at the distal section. An elongate stabilizer member is configured to extend from the stabilizer member lumen and provide lateral support to the distal end of the stabilizer sheath. A guide catheter having a shaped distal section that has a curved configuration in a relaxed state has an outer surface which is configured to move axially within a portion of the inner lumen of the stabilizer sheath that extends from the proximal end of the stabilizer sheath to the port. A tissue penetration member is configured to move axially within an inner lumen of the tubular guide catheter and is axially extendable from a distal end of the guide catheter for membrane penetration. The stabilizer sheath is advanced through a superior vena cava of the patient and positioned with the stabilizer member within the inferior vena cava. The port of the stabilizer sheath is positioned adjacent the right atrium of the patient's heart. The distal end of the guide catheter is advanced through the inner work lumen of the stabilizer sheath until the distal end of the guide catheter is positioned adjacent a desired site of the septum of the patient's heart. The tissue penetration member is advanced from the distal end of the guide catheter and activated. The tissue penetration actuator is then advanced distally through the septum.

In another embodiment, a transmembrane access system includes a guide catheter having a shaped distal section that includes a curved configuration in a relaxed state, an inner work lumen extending within a length thereof, a port disposed on a distal end of the catheter and in communication with the inner work lumen and a stabilizer member lumen which is substantially parallel to a nominal longitudinal axis of the guide catheter. The stabilizer member lumen extends proximally from a distal port of the stabilizer member lumen which is disposed proximal to the shaped distal section of the guide catheter. An elongate stabilizer member is configured to extend distally from the distal port of the stabilizer member lumen of the guide catheter and provide lateral support to the distal portion of the guide catheter. A tissue penetration member is configured to move axially within the inner work lumen of the guide catheter and is axially extendable from as distal end of the guide catheter for membrane penetration. In some embodiments, the system includes an elongate dilator configured to slide axially within the working lumen of the guide catheter and having a distal stabilizer member lumen configured to allow axial passage of the elongate stabilizer member. The distal stabilizer member lumen has a proximal port and distal port which are configured to extend beyond a distal end of the guide catheter.

In another embodiment of a method of accessing the left atrium of a patient's heart from the right atrium of the patient's heart, a transmembrane access system is provided, including a guide catheter having a shaped distal section that includes a curved configuration in a relaxed state, an inner work lumen extending therein, a port disposed on a distal end of the guide catheter and in communication with the inner work lumen and a stabilizer member lumen substantially parallel to a nominal longitudinal axis of the guide catheter proximal of the shaped distal section. An elongate stabilizer member is configured to extend from the stabilizer member lumen and provide lateral support to the distal end of the stabilizer sheath. A tissue penetration member is configured to move axially within the inner work lumen of the guide catheter and is axially extendable from the distal end of the guide catheter for membrane penetration. The guide catheter is advanced through a superior vena cava of the patient and positioned with the stabilizer member within the inferior vena cava. The port of the guide catheter is positioned adjacent a desired site of the septum of the patient's heart. The tissue penetration member is advanced from the distal port of the guide catheter and activated. The tissue penetration member is then advanced distally through the septum.

In another embodiment, a stabilized guide catheter system includes an elongate guide catheter having an inner work lumen and a distal port in fluid communication with the inner work lumen. The guide catheter has a shaped distal section that includes a curved configuration in a relaxed state, and a stabilizer member lumen substantially parallel to a longitudinal axis of the guide catheter. The stabilizer member lumen extends proximally from an intermediate port of the stabilizer member lumen which is disposed proximal to the shaped distal section of the guide catheter. The stabilizer member lumen also extends distally from the intermediate port to a distal port of the stabilizer member lumen which is disposed in the shaped distal section of the guide catheter. An elongate stabilizer member is configured to extend from the intermediate port and distal port of the stabilizer member lumen and provide lateral support to a distal portion of the guide catheter.

These features of embodiments will become more apparent from the following detailed description when taken in conjunction with the accompanying exemplary drawings.

### BRIEF DESCRIPTION OF THE DRAWINGS

FIG. 1 is an elevational view of an embodiment of a transmembrane access system.

FIG. 2 is an enlarged view in partial section of a side port portion of a stabilizer sheath of the transmembrane access system of FIG. 1 indicated by the encircled portion 2-2 of FIG. 1 and showing a distal portion of a guide catheter and an elongate tissue penetration device extending from the guide catheter.

FIG. 2A is an enlarged view of a tissue penetration member secured to a torquable shaft of the tissue penetration device, indicated by the encircled portion 2A-2A in FIG. 2.

FIG. 3 is an enlarged view in longitudinal section of the tissue penetration member and attachment of the tissue penetration member to the torquable shaft.

FIG. 3A is a transverse cross sectional view of the joint between the tissue penetration member and torquable shaft indicated by lines 3A-3A in FIG. 3.

FIG. 3B is an elevational view of the tissue penetration member and torquable shaft of the elongate tissue penetration device.

FIGS. 3C and 3D illustrate transverse cross sectional views of the elongate tissue penetration device taken along lines 3C-3C and 3D-3D of FIG. 3B, respectively,

FIG. 4 is an enlarged view in longitudinal section of the proximal adapters of a proximal portion of the transmembrane access system of FIG. 1.

FIG. 5 is an elevational view of the stabilizer sheath of the transmembrane access system of FIG. 1 with the curved distal section of the sheath lying in a plane which is orthogonal to the page.

FIG. 6 is an elevational view of the stabilizer sheath of FIG. 5 shown with the curved distal section lying in the plane of the page and with the proximal adapter not shown attached to the Luer connector fitting.

FIG. 7 is an enlarged transverse cross sectional view of the stabilizer sheath taken at the side port along lines 7-7 of FIG. 6.

FIG. 7A is a transverse cross sectional view of the stabilizer sheath taken along lines 7A-7A of FIG. 7.

FIG. 8 is an enlarged view in longitudinal section of the side port of the stabilizer sheath indicated by the encircled portion 8-8 in FIG. 6.

FIG. 8A is a perspective view of a reinforcement member of the side port section of the stabilizer sheath of FIG. 8.

FIG. 8B illustrates the side port section of an embodiment of the stabilizer sheath having an inflatable abutment.

FIG. 9 is an enlarged view in longitudinal section of a distal portion of the stabilizer sheath immediately distal of the side port indicated by the encircled portion 9-9 in FIG. 6 and illustrating the tapered characteristic of the distal portion of the stabilizer sheath.

FIG. 10 is an enlarged view in longitudinal section of the distal most portion of the stabilizer sheath indicated by the encircled portion 10-10 in FIG. 6 an illustrating the curled curvature or “pig tail” section of the distal most portion of the stabilizer sheath.

FIG. 11 is an enlarged view in longitudinal section of the proximal end portion of the stabilizer sheath indicated by the encircled portion 11-11 in FIG. 6 and illustrating the inner lumen of the stabilizer sheath and the Luer connector secured to the proximal end of the stabilizer sheath.

FIG. 12 illustrates the guide catheter of FIG. 1 showing the curved distal section of the guide catheter lying in the plane of the page with the guide catheter in a relaxed state.

FIG. 13 illustrates the guide catheter of FIG. 1 showing the shaped distal section of the guide catheter lying in a plane that is orthogonal to the page with the guide catheter in a relaxed state.

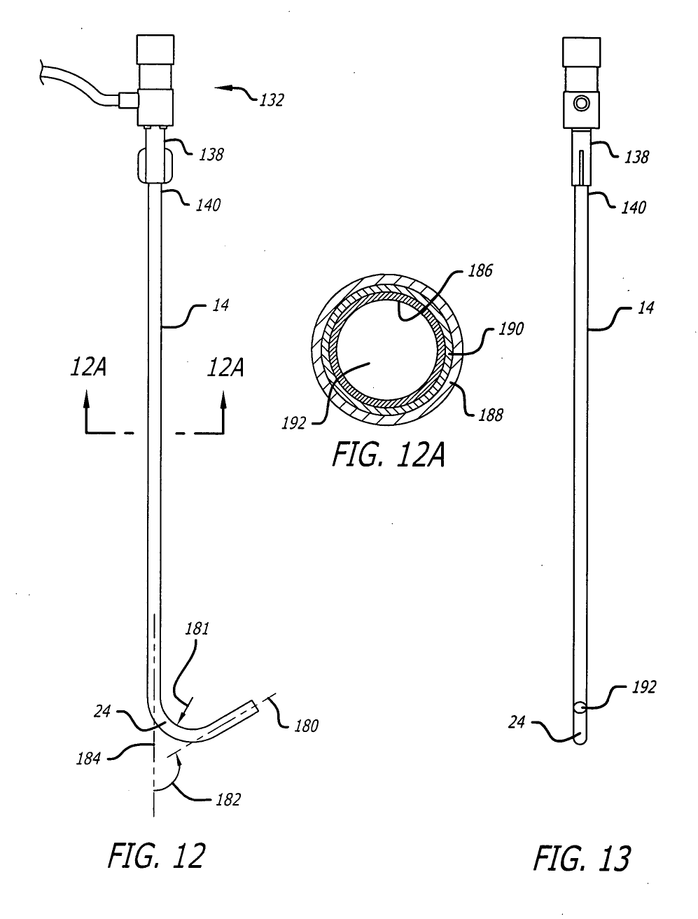

FIG. 12A illustrates a transverse cross sectional view of the guide catheter taken along lines 12A-12A of FIG. 12 and showing the braided layer of the guide catheter.

FIG. 14 illustrates an embodiment of an obturator sheath configured to be disposed within the inner lumen of the stabilizer sheath and block the side port of the stabilizer sheath to prevent damage to tissue adjacent the stabilizer sheath during insertion thereof.

FIG. 15 illustrates an enlarged view in longitudinal section of the obturator disposed within the side port of the stabilizer sheath and having a guidewire disposed within the inner lumen of the obturator sheath.

FIG. 16 is a transverse cross sectional view of the stabilizer sheath, obturator sheath and guidewire taken along lines 16-16 of FIG. 15.

FIG. 17 is an elevational view in longitudinal section of the distal end of the obturator sheath illustrating the tapered configuration of the distal end of the obturator sheath and showing the guidewire disposed within and extending from the inner lumen of the obturator sheath.

FIG. 17A illustrates an enlarged view in section of an embodiment of a side port configuration of an embodiment of a stabilizer sheath wherein the guidewire extending through the inner lumen of the stabilizer sheath embodiment is maintained in a concentric arrangement with the longitudinal axis of the stabilizer sheath by a sleeve portion that is also shaped within the side port to act as a deflective surface.

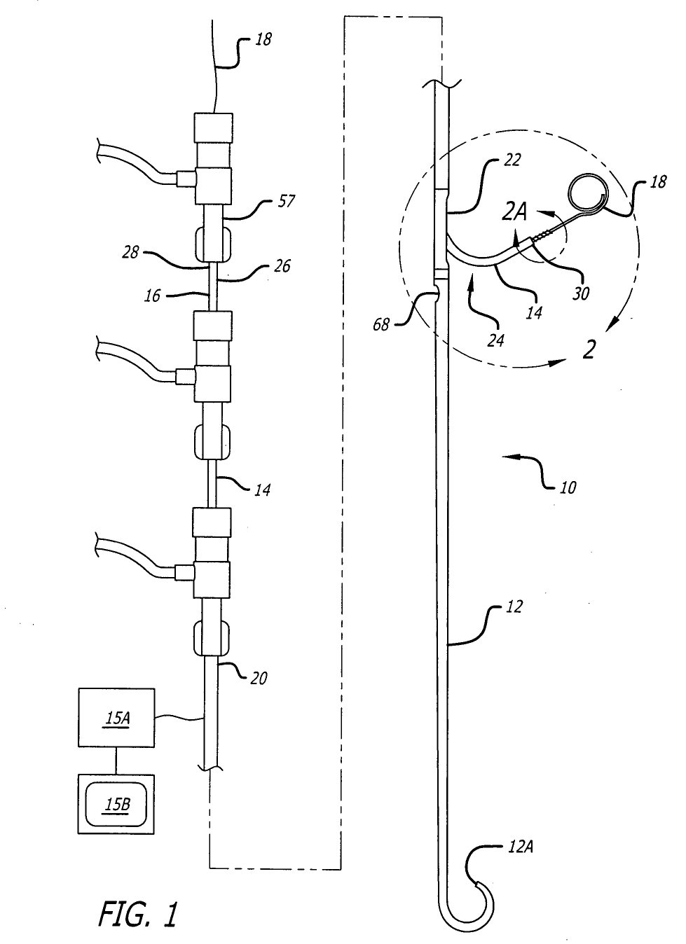

FIG. 18 shows a diagramatic view of the stabilizer sheath of the transmembrane access system of FIG. 1 being advanced into position over a guidewire with the distal end of the stabilizer sheath, which is being maintained is a straightened configuration by the guidewire, disposed within the inferior vena cava and the side port facing the right atrium of the patient. The obturator sheath is shown disposed within the inner lumen of the stabilizer sheath and is blocking the side port. The guidewire is also disposed within the inner lumen of the obturator sheath.

FIG. 19 shows an enlarged elevational view of the side port section of the stabilizer sheath after removal of the obturator sheath with the distal end of the guide catheter and the distal end of the tissue penetration device, disposed within the distal end of the guide catheter, being advanced distally through the inner lumen of the stabilizer sheath to the side port.

FIG. 20 shows the transmembrane access system with the elongate tissue penetration device disposed within the guide catheter which is disposed within the inner lumen of the stabilizer sheath. The guide catheter distal end is extending radially from the side port of the stabilizer sheath and is positioned adjacent a desired area of the septum for access.

FIG. 20A is an elevational view of a stylet having a shaped distal section that may be used within the inner lumen of the tissue penetration member.

FIGS. 20B-20D illustrate a tissue penetration sequence by the tissue penetration member through the septum of the patient.

FIG. 21 illustrates the tissue penetration member having been activated by rotation of the torquable shaft from a proximal portion of the torquable shaft and having penetrated the septal wall of the patient's heart with a guidewire having been extended through an inner lumen of the shaft into the left atrium of the patient's heart.

FIG. 22 is an enlarged view of the heart portion of FIG. 21 indicated by encircled portion 22 of FIG. 21.

FIG. 23 shows the guidewire in position across the septal wall with the distal end of the guidewire in position in the left atrium after the stabilizer sheath, guide catheter and elongate tissue penetration device have been withdrawn proximally.

FIGS. 24A-24C illustrate how the orientation of the distal end of the guide catheter can be controlled by advancing and retracting the guide catheter within the side port of the stabilizer sheath, axial movement of the stabilizer sheath relative to the right atrium and variation of the curvature of the shaped distal section of the guide catheter.

FIGS. 25 and 26 illustrate a method of transmembrane access across a patient's septal wall by using an embodiment of a guide catheter and elongate tissue penetration device having a tissue penetration member activated by rotation without the use of a stabilizer sheath.

FIG. 27 is an elevational view of an embodiment of a transmembrane access system that includes a proximal activation modulator.

FIG. 28 is an enlarged view in partial section of a side port portion of a stabilizer sheath of the transmembrane access system of FIG. 27 indicated by the encircled portion 28-28 of FIG. 27.

FIG. 29 is an enlarged view of a tissue penetration member secured to a torquable shaft of a tissue penetration device of the system, indicated by the encircled portion 29-29 in FIG. 27.

FIG. 29A is an enlarged view of another embodiment of a tissue penetration member having two helical tissue penetration members.

FIG. 30 is a perspective view of an embodiment of an activation modulator for applying controlled axial movement to the tissue penetration member and limiting the rotational movement of the tissue penetration member.

FIG. 31 is an exploded view of the activation modulator and proximal section of the torquable shaft of the transmembrane access system of FIG. 27.

FIG. 32 is an enlarged view of a distal portion of a threaded inner barrel of the activation modulator.

FIG. 33 is an elevational view of the activation modulator embodiment of FIG. 30.

FIG. 34 is an elevational view in longitudinal section of the activation modulator of FIG. 33 taken along lines 34-34 of FIG. 33 showing the threaded inner barrel disposed at a proximal limit of axial movement.

FIG. 35 is an enlarged view of a rotation seal disposed about the threaded inner barrel of the activation modulator indicated by the encircled portion 35-35 of FIG. 34.

FIG. 36 is an elevational view in longitudinal section of the activation modulator of FIG. 34 with the threaded inner barrel disposed at a distal limit of axial movement.

FIG. 37 is an elevational view, partially broken away, of an embodiment of a tissue penetration device.

FIG. 38 is an enlarged view in longitudinal section of the tissue penetration device of FIG. 37 indicated by the encircled portion 38-38 in FIG. 37.

FIG. 39 is an enlarged view in longitudinal section of the tissue penetration device of FIG. 37 indicated by the encircled portion 39-39 in FIG. 37.

FIG. 40 is an elevational view, partially broken away, of another embodiment of a tissue penetration device.

FIG. 41 illustrates a distal portion of a tubular needle of the tissue penetration device of FIG. 40 which has a series of alternating partial transverse cuts in the tubular member to enhance the flexibility of the distal portion of the tubular needle.

FIG. 42 is an enlarged view in longitudinal section of the tissue penetration device of FIG. 37 indicated by the encircled portion 42-42 in FIG. 40.

FIG. 43 is an elevational view of an embodiment of a transmembrane access system that has a pigtail tip of a stabilizer sheath laterally curling away from and extending opposite the side port of a stabilizer sheath of the system.

FIG. 44 is an enlarged view of the transmembrane access system of FIG. 43 taken along the encircled portion 44-44 of FIG. 43.

FIG. 45 is an enlarged view of the transmembrane access system of FIG. 44, without the tissue penetration device shown, illustrating the ultrasound energy propagation of the ultrasound transducers disposed on the stabilizer sheath.

FIG. 46 is an elevational view of another embodiment of a transmembrane access device having a stabilizer sheath with a guidewire extending from a distal end of the stabilizer sheath for lateral support of the stabilizer sheath and a guide catheter extending distally from a distal port of the stabilizer sheath.

FIG. 47 is an enlarged view of the transmembrane access system of FIG. 46 taken along the encircled portion 47-47 of FIG. 46.

FIG. 48 illustrates the transmembrane access system of FIG. 46 with the distal end of the stabilizer sheath disposed within a vena cava of a patient and a distal end of the guide catheter and tissue penetration device of the system extending from the vena cava and into the right atrium (not shown).

FIG. 49 shows another embodiment of a stabilizer sheath of the transmembrane access system of FIG. 46 wherein the guidewire used to stabilize the distal section of the stabilizer sheath is slidingly disposed within a short lumen at a distal section of the stabilizer sheath.

FIG. 50 illustrates an embodiment of a stabilized guide catheter having a separate stabilizer member lumen with a distal port disposed proximal to a shaped distal section of the guide catheter and a stabilizer member disposed in the stabilizer member lumen and extending from the distal port for stabilizing the distal section of the guide catheter.

FIG. 51 is a transverse cross section of the stabilized guide catheter of FIG. 50 taken along lines 51-51 of FIG. 50.

FIG. 52 is an enlarged view of a distal portion of the stabilized guide catheter of FIG. 50 taken along the encircled portion 52-52 of FIG. 50.

FIG. 53 shows a transmembrane access system including the stabilized guide catheter embodiment of FIG. 50 disposed within a vena cava of a patient with the distal end of the guide catheter extending from the vena cava and into the right atrium (not shown) and a distal portion of a tissue penetration device extending from a distal end of the stabilized guide catheter.

FIG. 54 shows an enlarged view of a distal portion of another embodiment of a stabilized guide catheter of FIG. 50 wherein an elongate stabilizer member is slidingly disposed within a short lumen at a distal portion of the stabilized guide catheter. The short stabilizer member lumen has a proximal port and a distal port. The distal port is disposed proximal to the shaped distal section of the stabilized guide catheter for the embodiment shown.

FIG. 55 shows another embodiment of a stabilized guide catheter wherein a stabilizer member is disposed within a stabilizer member lumen which has a distal port at its distal end and an intermediate port located a predefined distance proximal to the distal end of the stabilized guide catheter. In the embodiment shown, the intermediate port of the stabilizer member lumen is disposed just proximal of the shaped distal section of the guide catheter.

FIG. 56 shows the stabilized guide catheter embodiment of FIG. 55, in which the stabilizer member has been withdrawn from a portion of the stabilizer member lumen disposed distal of the intermediate port, allowing the stabilized guide catheter to assume its relaxed, curved configuration in the shaped distal section of the stabilized guide catheter. The stabilizer member has been re-advanced through the intermediate port to provide lateral support to the shaped distal section of the stabilized guide catheter.

FIG. 57 shows another embodiment of a stabilized guide catheter, in which an elongate dilator having a distal stabilizer member lumen is provided. The dilator is slidably disposed within the guide catheter and the distal stabilizer member lumen is positioned such that both the proximal and distal port of the distal stabilizer member lumen can extend beyond the distal end of the guide catheter. This allows a stabilizer member to enter the proximal port of the distal stabilizer member lumen and exit the elongate dilator at its distal tip through the distal port of the distal stabilizer member lumen.

FIG. 58 illustrates a transverse cross section of the stabilized guide catheter of FIG. 57 taken along lines 58-58 of FIG. 57.

FIG. 59 is a transverse cross section of the stabilized guide catheter of FIG. 57 taken along lines 59-59 of FIG. 57.

FIG. 60 is an enlarged view of a distal portion of the stabilized guide catheter of FIG. 57 indicated by the encircled portion 60-60 of FIG. 57.

FIG. 61 shows the transmembrane access system of FIG. 57, wherein the stabilized guide catheter and elongate dilator have been advanced over a stabilizer member into a desired location.

FIG. 62 shows the transmembrane access system of FIG. 57, wherein the stabilizer member has been withdrawn from the dilator and the stabilizer member has been re-advanced through a distal port of the stabilizer member lumen to provide lateral support for the shaped distal section of the stabilized guide catheter.

FIG. 63 shows the transmembrane access system of FIG. 57 wherein the dilator has been withdrawn proximally and the distal end of the stabilized guide catheter is disposed within a desired site.

### DETAILED DESCRIPTION

Embodiments are directed to systems and methods for accessing a second side of a tissue membrane from a first side of a tissue membrane. In more specific embodiments, devices and methods for accessing the left atrium of a patient's heart from the right atrium of a patient's heart are disclosed. Indications for such access devices and methods can include the placement of cardiac monitoring devices, transponders or leads for measuring intracardiac pressures, temperatures, electrical conduction patterns and voltages and the like. The deployment of cardiac pacemaker leads can also be facilitated with such access devices and methods. Such access may also be useful in order to facilitate the placement of mitral valve repair devices and prosthetics, as well as other indications.

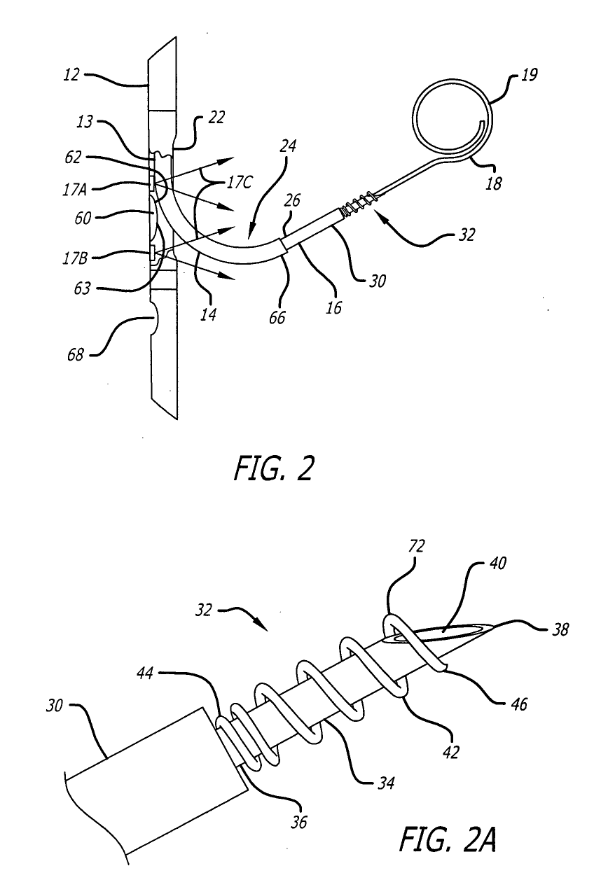

FIG. 1 illustrates an embodiment of a transmembrane access system 10. The system 10 shown in FIG. 1 includes a stabilizer sheath 12, a guide catheter 14, an elongate tissue penetration device 16 and a guidewire 18 disposed within an inner lumen of the elongate tissue penetration device 16. The stabilizer sheath 12 has a tubular configuration with an inner lumen 13, shown in FIG. 2, extending from a proximal end 20 of the stabilizer sheath 12 to a side port 22 disposed in the sheath 12. In one embodiment, the inner lumen 13 extends to the distal port 12A of the stabilizer sheath 12, and is open to one or more side ports 22 at one or more locations between the proximal and distal ends. The guide catheter 14 has a tubular configuration and is configured with an outer surface profile which allows the guide catheter 14 to be moved axially within the inner lumen 13 of the stabilizer sheath 12. The guide catheter 14 has a shaped distal section 24 with a curved configuration in a relaxed state which can be straightened and advanced through the inner lumen 13 of the stabilizer sheath 12 until it exits the side port 22 of the stabilizer sheath 12 as shown in more detail in FIG. 2.

An optional ultrasound energy generator or ultrasound emission member and ultrasound receiver, which may be separate elements or combined in the form of an ultrasound transducer, may be disposed on the access system 10 so as to allow visualization or imaging of the space surrounding the system 10 during a procedure. FIG. 1 shows an ultrasound signal controller 15A in communication with a display member in the form of a video monitor 15B. The ultrasound signal controller 15 is also in communication with a first ultrasound transducer 17A and a second ultrasound transducer 17B, shown in FIG. 2. Ultrasound energy can be emitted from the ultrasound transducers 17A and 17B in the form of an ultrasound signal and projected into the space surrounding the access system 10 during use. The ultrasound energy reflected from the surrounding tissue and space may then be received by the transducers 17A and 17B and converted into information, such as imaging information, that may then be used for positioning the access system 10, or any other suitable use. Information such as the tissue contour of target tissue, the thickness of the membrane to be penetrated, or the distance to target tissue from the distal tip of the guide catheter 14 or tissue penetration device 16 can be determined and optionally displayed on the display member or video monitor 15B.

In the embodiment shown in FIGS. 1 and 2, the transducers 17A and 17B are configured to project an ultrasound signal in a direction that is substantially radially outward from the stabilizer sheath 12 in the direction of the opening of the side port 22, as shown in FIG. 2 by arrows 17C. Ultrasound transducer 17A may emit an ultrasound signal through the side port 22 without obstruction, so long as the guide catheter is not disposed in the side port 22, as it is located opposite the side port 22. If the guide catheter 14 is disposed within the side port 22, the ultrasound transducers can transmit and receive ultrasound signals through the guide catheter 14. Ultrasound transducer 17B may also transmit and receive ultrasound signals through a side wall of the stabilizer sheath 12. The ultrasound transducers 17A and 17B may also be disposed on an outer surface of the stabilizer sheath 12, and in some embodiments, disposed on an outer surface of the stabilizer sheath 12 adjacent the side port 22. The ultrasound transducers 17A and 17B may be any of a variety of suitable types, including piezoelectric, phased array or the like. Embodiments of ultrasound energy emission members may include ultrasound generating components of devices such as piezoelectric transducers but may also include any suitable type of ultrasound emitting member such as a vibrating member activated by a remote ultrasound energy source, rotating ultrasound mirror or reflective device or the like.

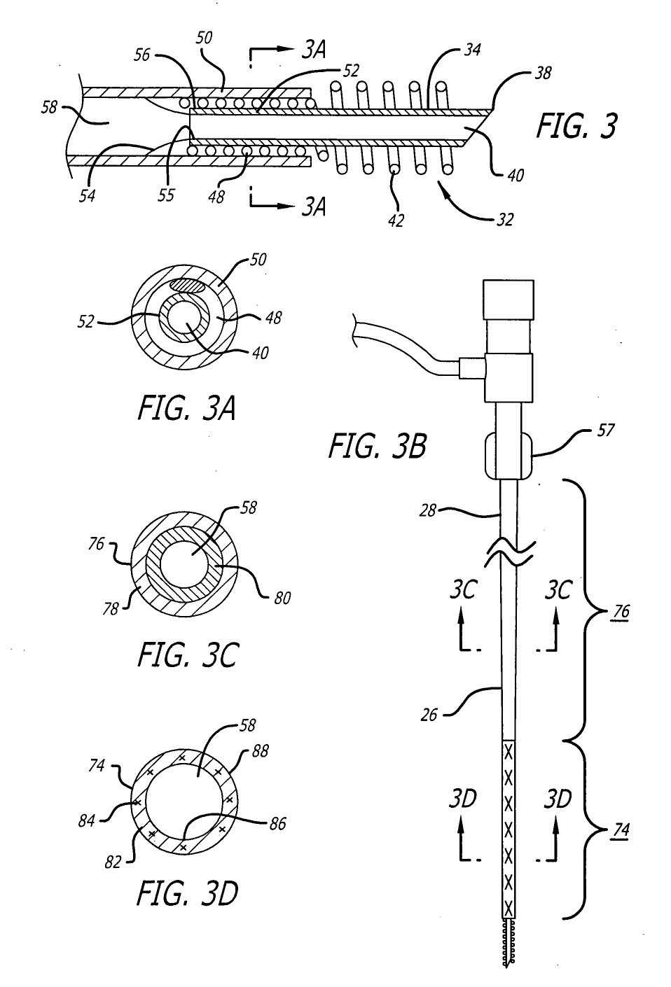

The elongate tissue penetration device 16 includes a tubular flexible, torquable shaft 26 having a proximal end 28, shown in FIG. 1, and a distal end 30. The distal end 30 of the torquable shaft 26 is secured to a tissue penetration member 32, shown in FIG. 2A, which is configured to penetrate tissue upon activation by rotation of the tissue penetration member 32. The tissue penetration member 32 has a tubular needle 34 with a proximal end 36, a sharpened distal end 38 and an inner lumen 40 that extends longitudinally through the tubular needle 34. A helical tissue penetration member 42 has a proximal end 44 and a sharpened distal end 46 and is disposed about the tubular needle 34. The helical tissue penetration member 42 has an inner diameter which is larger than an outer diameter of the tubular needle 34 so as to leave a gap between the tubular needle 34 and the helical tissue penetration member 42 for the portion of the helical tissue penetration 42 that extends distally from the distal end 30 of the torquable shaft 26. Referring to FIG. 3, a proximal portion 48 of a coil of the helical tissue penetration member 42 is secured to a distal portion 50 of the inner lumen of the tubular torquable shaft 26 and a proximal portion 52 of the tubular needle 34 is secured to the proximal portion 48 of the coil of the helical tissue penetration member 42. A conical ramp 54 may be disposed at the proximal end 56 of the tubular needle 34 in order to form a smooth transition from the inner lumen 58 of the tubular torquable shaft 26 to the inner lumen 40 of the tubular needle 34 which facilitates guidewire 18 movement therethrough. The proximal end 56 of the tubular needle 34 may also have a tapered section 55 formed or machined into the inner surface of the tubular needle 34. Guidewire 18 that may be used in conjunction with the tissue penetration device 16 may be an Inoue wire, manufactured by TORAY Company, of JAPAN. This type of guidewire 18, such as the Inoue CMS-1 guidewire, may have a length of about 140 cm to about 260 cm, more specifically, about 160 cm to about 200 cm. The guidewire 18 may have a nominal transverse outer dimension of about 0.6 mm to about 0.8 mm. The distal section 19 of this guidewire 18 embodiment may be

Referring to FIG. 2, an abutment 60 having a radially deflective surface 62 is disposed within the inner lumen 13 of the stabilizer sheath 12 opposite the side port 22 of the sheath 12. In the embodiment shown, the apex 63 of the abutment 60 is disposed towards the distal end of the side port 22 which disposes the deflective surface 62 in a position which is longitudinally centered in the side port 22. This configuration allows for reliable egress of the distal end 66 of the guide catheter 14 from the side port 22 after lateral deflection of the guide catheter 14 by the deflective surface 62. The deflective surface 62 of the abutment 60 serves to deflect the distal end 66 of the guide catheter 14 from a nominal axial path and out of the side port 22 during advancement of the guide catheter 14 through the inner lumen 13 of the stabilizer sheath 12. The abutment 60 may be a fixed mass of material or may be adjustable in size and configuration. In one embodiment the abutment 60 is inflatable and has an inflation lumen extending proximally through the stabilizer sheath 12 from the inflatable abutment to the proximal end 20 of the stabilizer sheath 12. An optional guidewire exit port 68 may be disposed in the wall of the stabilizer sheath 12 in fluid communication with a distal guidewire port 12A of the stabilizer sheath 12 and the inner lumen 13 of the stabilizer sheath 12. The optional guidewire exit port 68 is disposed distally of the side port 22 and proximally of the distal port 12A. Such a configuration allows the stabilizer sheath 12 to be advanced into position over a guidewire (not shown) disposed within the inner lumen 13 between distal port 12A and exit port 68. with the guide catheter 14 and elongate tissue penetration device 16 disposed in the inner lumen 13 of the stabilizer sheath 12 proximally of side port 22. A standard guidewire may also be disposed in the distal guidewire port 12A of the stabilizer sheath 12 and extend proximally in the inner lumen 13 of the stabilizer sheath 12 to the proximal end 20 of the sheath 12.

The elongate tissue penetration device 16, as shown in more detail in FIGS. 3-3D, includes the tubular torquable shaft 26 secured to the tissue penetration member 32 at a distal end of the tubular torquable shaft 26 and a Luer fitting 57 at the proximal end 28 of the shaft 26. FIGS. 3 and 3A illustrate an enlarged view in section of the junction between the tissue penetration member 32 and the tubular torquable shaft 26. As shown, the proximal portion 48 of the coil of the helical tissue penetration member 42 is secured to the distal portion 50 of the inner lumen 58 of the tubular torquable shaft 26 by an adhesive. Adhesives such as epoxy, UV epoxy or polyurethane may be used. Other suitable methods of joining the helical tissue penetration member 42 to the tubular torquable shaft 26 may include soldering, welding or the like. The proximal portion 52 of the tubular needle 34 is secured to the proximal portion 48 of the helical tissue penetration member 42 in a substantially concentric arrangement also by an adhesive that may be the same as or similar to those discussed above. The conical ramp 54 is disposed at the proximal end 56 of the tubular needle 34 in order to form a smooth transition from the inner lumen 58 of the tubular torquable shaft 26 to the inner lumen 40 of the tubular needle 34 and may be formed of a polymer or epoxy material. The distal end 46 of the helical tissue penetration member 42 has a sharpened tip 38 in order to facilitate tissue penetration upon rotation and advancement of the tissue penetration member 32.

The outer transverse dimension or diameter of the helical tissue penetration member 42 may be the same as or similar to an outer transverse dimension or diameter of the tubular torquable shaft 26. Alternatively, the outer transverse dimension or diameter of the helical tissue penetration member 42 may also be greater than the nominal outer transverse dimension of the tubular torquable shaft 26. The outer transverse dimension of an embodiment of the helical tissue penetration member 42 may also taper distally to a larger or smaller transverse dimension.

The helical tissue penetration member 42 can have an exposed length distally beyond the distal end 30 of the torquable shaft 26 of about 4 mm to about 15 mm. The inner transverse diameter of the coil structure of the helical tissue penetration member 42 can be from about 0.5 mm to about 2.5 mm. The pitch of the coil structure may be from about 0.3 mm to about 1.5 mm of separation between axially adjacent coil elements of the helical tissue penetration member 42. In addition, helical tissue penetration member embodiments may include coil structures having multiple elongate wire coil elements 72 that can be wound together. The elongate wire element 72 may have an outer transverse dimension or diameter of about 0.02 mm to about 0.4 mm. The helical tissue penetration member can be made of a high strength material such as stainless steel, nickel titanium alloy, MP35N, Elgiloy or the like. The elongate coiled element 72 may also be formed of a composite of two or more materials or alloys. For example, one embodiment of the elongate coiled element 72 is constructed of drawn filled tubing that has about 70 percent to about 80 percent stainless steel on an outer tubular portion and the remainder a tantalum alloy in the inner portion of the element. Such a composition provides high strength for the helical tissue penetration member 42 is compatible for welding or soldering as the outer layer of material may be the same or similar to the material of the braid of the torquable shaft 26 or the tubular needle 34. Such a drawn filled configuration also provides enhanced radiopacity for imaging during use of the tissue penetration device 16.

The tubular needle 34 of the tissue penetration member 34 may be made from tubular metallic material, such as stainless steel hypodermic needle material. The outer transverse dimension of an embodiment of the tubular needle 34 may be from about 0.25 mm to about 1.5 mm and the inner transverse dimension or diameter of the inner lumen 40 of the tubular needle 34 may be from about 0.2 mm to about 1.2 mm. The wall thickness of the tubular needle 34 may be from about 0.05 mm to about 0.3 mm. The tubular needle 34 may be made from other high strength materials such as stainless steel, nickel titanium alloy, MP35N, monel or the like.

The tubular torquable shaft 26 has a distal section 74 and a proximal section 76 as shown in FIG. 3B. The proximal section 76 of the shaft 26 has a tubular polymer layer 78 disposed about a high strength tubular member 80. The tubular polymer layer 78 may be made from materials such as Pebax, polyurethane, or the like. The material of the tubular polymer layer 78 may have a hardness of about 25D shore hardness to about 75D shore hardness. The high strength tubular member 80 may be made from materials such as stainless steel, nickel titanium alloy, MP35N, monel or the like. The distal section 74 of the tubular torquable shaft 26 may be constructed from a tubular polymer 82 similar to that of the proximal section 76 which is reinforced by a braid 84 of high strength material that provides torquability to the distal section 74 while maintaining the flexibility of the distal section 74. The reinforcing braid 84 may be disposed on an inside surface 86 or outside surface 88 of the tubular polymer material 82 of the distal section 74. Alternatively, the reinforcing braid 84 may also be embedded in the tubular polymer material 82 of the distal section 74 as shown in FIG. 3D. The elongate tissue penetration device 16 may have an overall length of about 3 mm to about 20 mm, more specifically, about 4 mm to about 12 mm. Alternative embodiments of the torquable shaft 26 can be a single composite extrusion of plastic and high strength braid with a varying durometers polymer along its length so that the torquable shaft 26 is flexible at the distal end and rigid at the proximal end of the torquable shaft 26.

Although the access system 10 is shown including tissue penetration device 16 which utilizes rotational energy for activation, other types of tissue penetration devices may also be used with the stabilizer sheath 12 and guide catheter 14 combination. For example, a tissue penetration device, such as the access catheters disclosed in commonly owned U.S. patent application Ser. No. 10/889,319, filed Jul. 12, 2004, titled “Methods and Devices for Transseptal Access”, which is hereby incorporated by reference herein in its entirety. For this example, the access catheters 14 and 110 disclosed in the above incorporated application could be substituted for the tissue penetration device 16 in the present application.

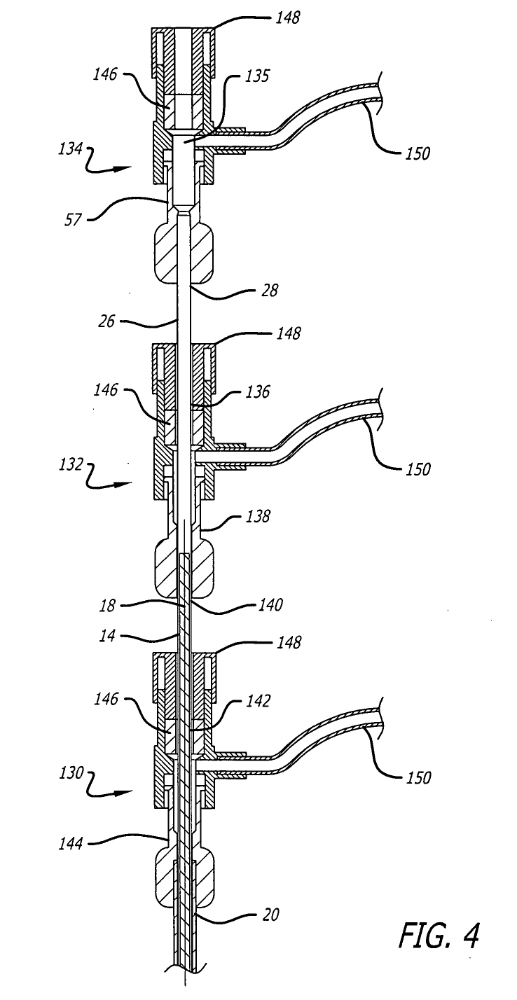

FIG. 4 is an enlarged view in longitudinal section of the proximal adapters 130, 132 and 134 of the proximal portion of the transmembrane access system 10 shown in FIG. 1. The guidewire 18 is not shown for clarity of illustration. Proximal adapter 134, having inner lumen 135, is secured to the Luer fitting 57 on the proximal end 28 of the tubular torquable shaft 26 of the elongate tissue penetration device 16. The elongate tissue penetration device 16 passes through an inner lumen 136 of proximal adapter 132 which is secured to a Luer fitting 138 secured to a proximal end 140 of the guide catheter 14. The guide catheter 14 and elongate tissue penetration device 16 are disposed within an inner lumen 142 of proximal adapter 130 which is secured to a Luer fitting 144 secured to the proximal end 20 of the stabilizer sheath 12. The proximal adapters 130, 132 and 134 all have inner lumens 135, 136 and 142 which allow for passage of appropriately sized devices while maintaining a seal between the devices and the inner lumens 135, 136 and 142. Each proximal adapter includes a resilient annular seal 146 that may be compressed by a threaded compression cap 148 so as to constrict the seal and form a seal around an outside surface of a catheter or other device disposed within an inner lumen of the seals 146. Each proximal adapter 130, 132 and 134 is also configured with a side port 150 in fluid communication with the respective inner lumens 135, 136 and 142 of the proximal adapters to allow for aspiration and flushing of the inner lumen, injection of contrast material, measurement of fluid pressure and the like. A proximal adapter embodiment suitable for use with embodiments 130, 132 and 134 of the system 10 can include the Toughy Borst made by Martek Company or commercially available hemostasis valves, including rotating hemostasis valves.

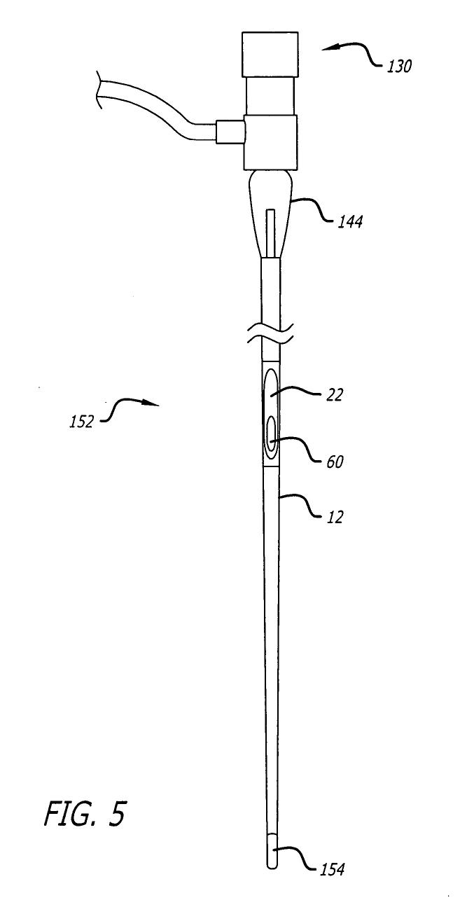

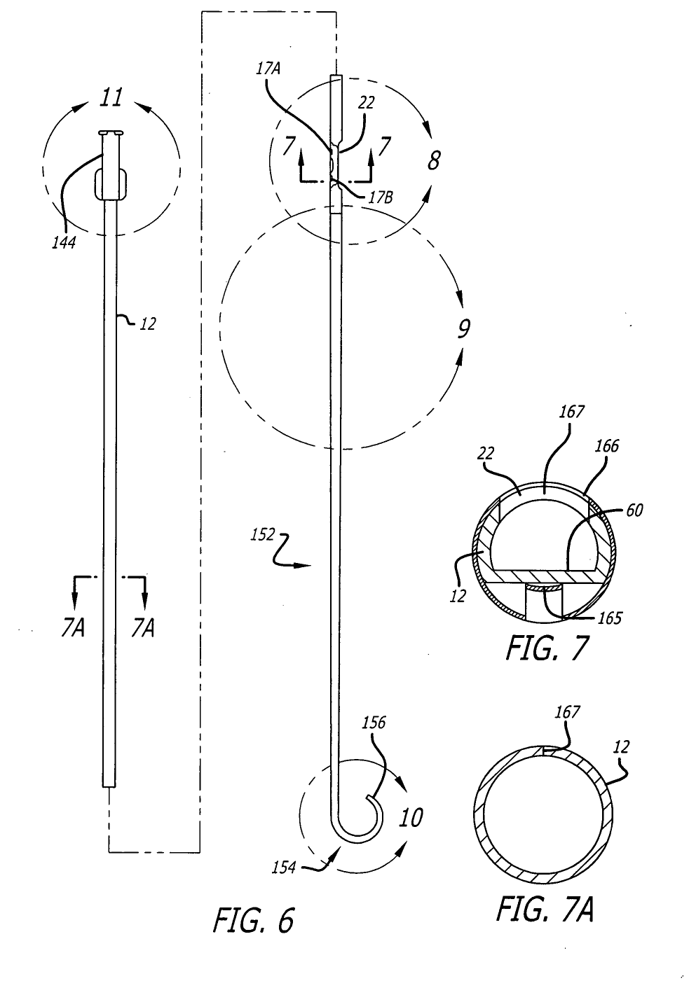

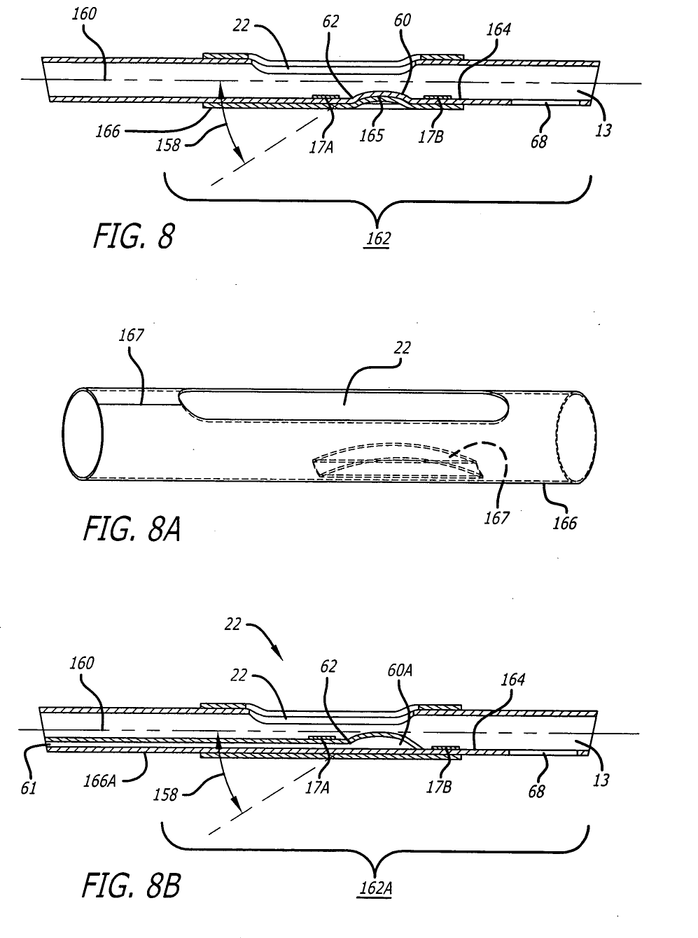

FIGS. 5-11 illustrate the stabilizer sheath 12 in more detail. The stabilizer sheath 12 has a substantially tubular configuration with a distal section 152 that tapers to a reduced transverse dimension or diameter and includes a pigtail or curled section 154 at the distal end 156 of the sheath 12 to avoid undesirable entry into small vessels and reduce vascular trauma. The side port 22, detailed in FIGS. 7 and 8, includes the abutment 60 having the radially deflective surface 62 disposed within the inner lumen 13 of the stabilizer sheath 12 opposite the side port 22 of the sheath 12. The deflective surface 62 forms an approximate angle 158 with the nominal longitudinal axis 160 of the side port section 162 of the stabilizer sheath 12 and extends radially inward from the nominal surface 164 of the inner lumen 13 of the stabilizer sheath 12. The deflective surface 62 of the abutment 60 serves to deflect the distal end 66 of the guide catheter 14 out of the side port 22 during advancement of the guide catheter 14 through the inner lumen 13 of the stabilizer sheath 12. The optional guidewire exit port 68 may be disposed in the wall of the stabilizer sheath 12 distal of the side port 22 and may be in fluid communication with a distal guidewire port 12A of the stabilizer sheath 12.

The side port 22 is configured to allow egress of the distal section 24 of the guide catheter 14 and elongate tissue penetration device 16. The side port 22 may have an axial or longitudinal length of about 10 mm to about 20 mm. The side port 22 may a width of about 1.5 mm to about 4 mm. The side port section 162 of the stabilizer sheath 12 may also include a reinforcement member 166 that strengthens the side port section 162 of the sheath 12 where material of the sheath 12 has been removed in order to create the side port 22. The reinforcement member 166 as well as the stabilizer sheath 12 optionally includes a peel away tear line 167 shown in FIGS. 7 and 7A that extends from the side port 22 of the stabilizer sheath 12 proximally to the proximal Luer fitting 144. The tear line 167 provides a fluid tight but weakened fault line that allows the stabilizer sheath to be removed from the patient's body without removal of the tissue penetration device 16 disposed within the inner lumen of the stabilizer sheath 12 when the tissue penetration device is positioned within the patient's body. The proximal adapter 130 and proximal Luer fitting 144 may also include a peel away tear line (not shown) in order to facilitate peel away removal of the stabilizer sheath 12.

The reinforcement member 166 may have a feature integrated within to collapse a portion of the inner lumen of the stabilizer sheath 12 and create the abutment or ramp 60. In another embodiment, a component, such as a dowel pin section or the like, can be trapped between the inner wall of the reinforcement member 166 and the outer wall of the stabilizer sheath 12 or an adhesive can be placed on the inner wall of the stabilizer sheath 12. The reinforcement member 166 shown in FIGS. 8 and 8A includes a deflected section 167 that displaces the wall of the stabilizer sheath 12 to create the abutment 60. The reinforcement member 166 may be made from a section of high strength tubular material bonded or secured to the outer surface of the stabilizer sheath 12 that is cut to an outline that matches the side port 22 of the sheath 12. The reinforcement member 166 may have a length of about 15 mm to about 30 mm. The reinforcement member 166 may have a wall thickness of about 0.05 mm to about 0.2 mm. The reinforcement member 166 may be made from any suitable high strength material such as stainless steel, nickel titanium alloy, MP35N, Elgiloy, composites such as carbon fiber composites, or the like.

The abutment 60 may be a fixed mass of material or may be adjustable in size and configuration. In one embodiment, the abutment 60 is inflatable and has an inflation lumen extending proximally through the stabilizer sheath 12 from the inflatable abutment 60 to the proximal end 20 of the stabilizer sheath 12. FIG. 8A illustrates the side port section 162 of an embodiment of the stabilizer sheath 12 having an inflatable abutment 60A that may be inflated for varying sizes by injection of an inflation fluid, gas or the like through an inflation lumen 61. The inflatable abutment 60A may be made from a compliant or non-compliant material. For inflatable abutment embodiments made from compliant materials, such as elastomers, the size of the abutment 60A may be adjusted by the amount of expansion or distention of the abutment 60A which could be controlled by the pressure level of the inflation substance. The side port section 162A includes a reinforcement member 166A that does not include a deflected section 167 as shown on the reinforcement member 166 discussed above.

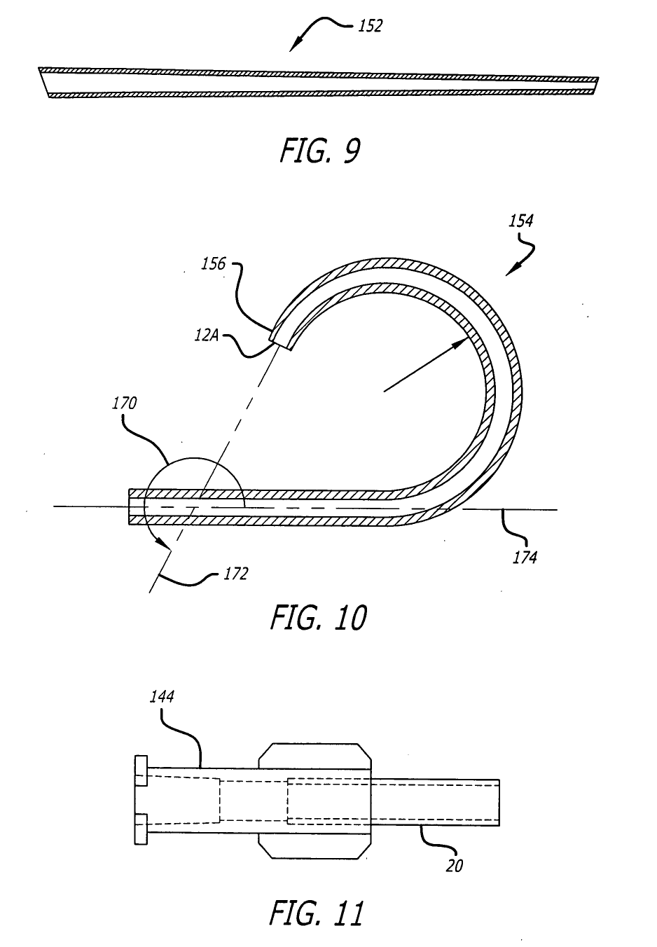

FIG. 9 illustrates the tapered characteristic a distal section 152 of the stabilizer sheath 12 immediately distal of the side port 22. The outer transverse dimension or diameter of the stabilizer sheath 12 may taper continuously from the side port 22 to the distal end 156 of the sheath 12. The inclusive taper angle of the sheath 12 over this distal section may be from about 0.1 degrees to about 5.0 degrees The nominal outer transverse dimension or diameter of the stabilizer sheath 12 may be from about 2.5 mm to about 6.0 mm, specifically, from about 3 mm to about 4 mm. The inner transverse dimension or diameter of the inner lumen 13 of the stabilizer sheath between the side port 22 and the Luer fitting 144, which is sized to accept the outer dimension of the guide catheter 14, may be from about 2.0 mm to about 5.0 mm. The Luer fitting 144 is secured to the proximal end 20 of the sheath by any suitable bonding method such as adhesive bonding, welding or the like. The Luer fitting 144 and joint between the Luer fitting 144 and proximal end 20 of the sheath 12 is shown in FIG. 11.

The distal end 156 of the stabilizer sheath 12 can include the curled section 154 having curvature or a “pig tail” arrangement which produces an atraumatic distal end 156 of the stabilizer sheath 12 while positioned within a patient's anatomy. The curled section 154 may have a radius of curvature of about 3 mm to about 12 mm and may have an angle of curvature 170 between a discharge axis 172 of the distal end 156 of the stabilizer sheath 12 and the nominal longitudinal axis 174 of the stabilizer sheath 12 of about 200 degrees to about 350 degrees. For the embodiment shown in FIG. 1, the curled section 154 is curled laterally in the same direction as the direction of the opening of the side port 22. The inner transverse dimension of the inner lumen 13 of the sheath 12 at the distal end 156 of the sheath 12 may be from about 0.5 mm to about 1.6 mm. The overall length of the stabilizer sheath 12 may be from about 40 cm to about 100 cm. The distance from the side port 22 to the distal end 156 of the sheath 12 may be from about 30 cm to about 65 cm. The stabilizer sheath 12 may be made from any suitable flexible material which is biocompatible, such as Pebax, polyurethane, polyethylene, and the like.

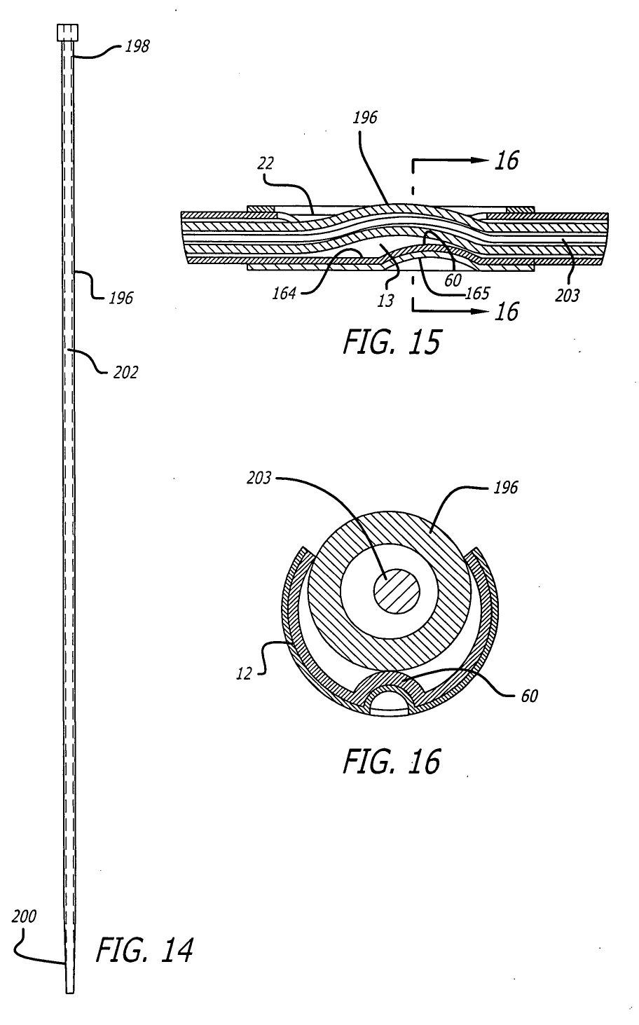

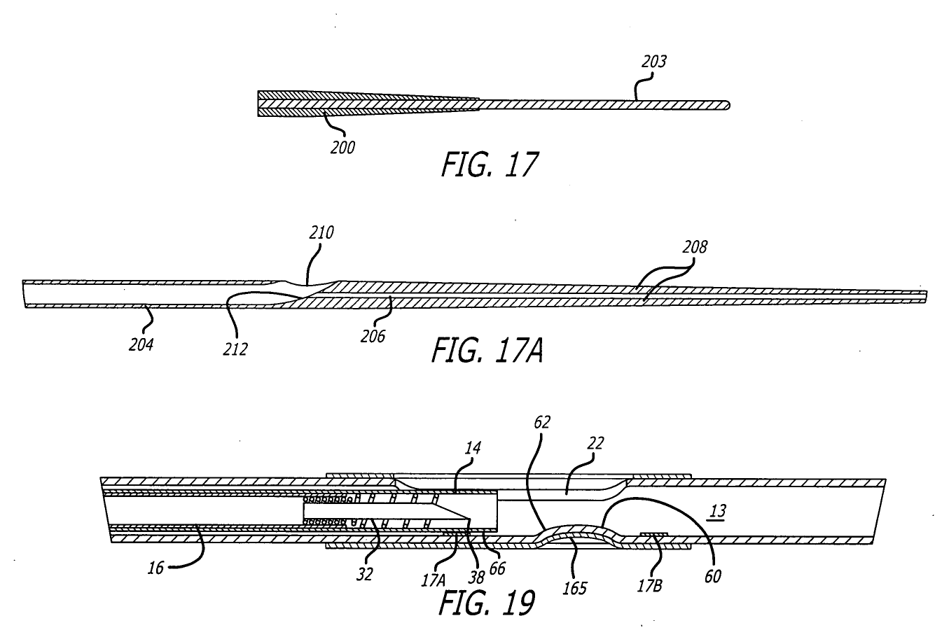

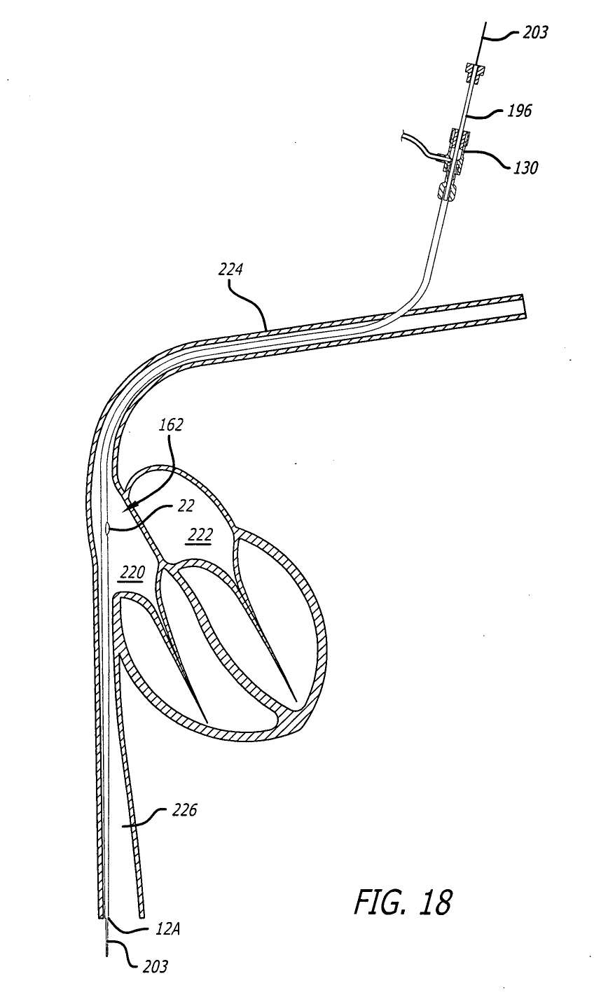

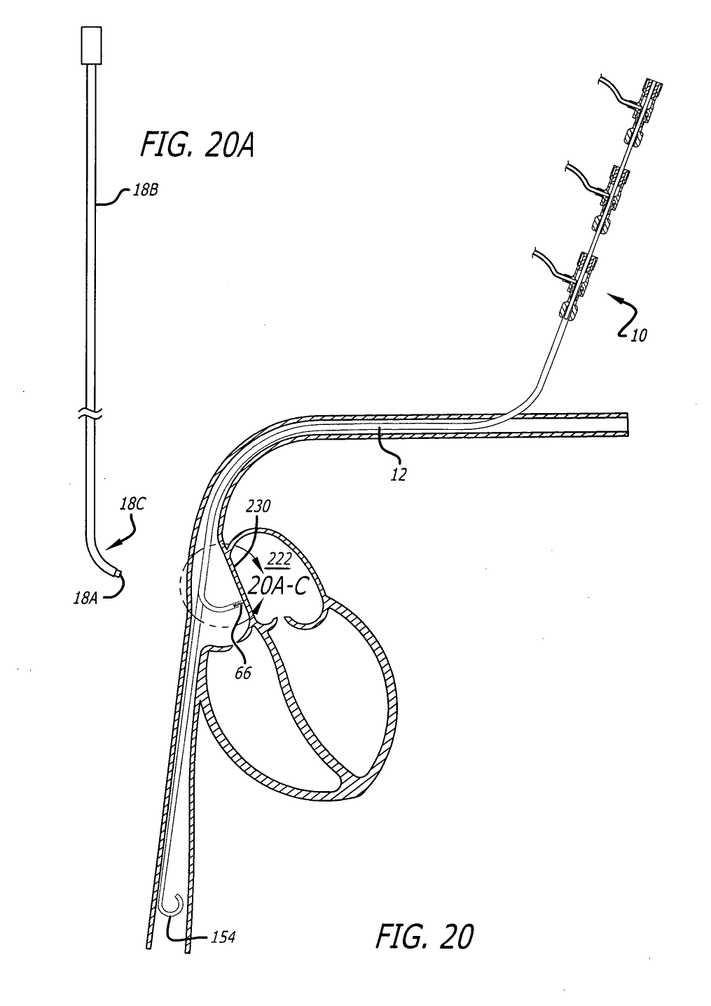

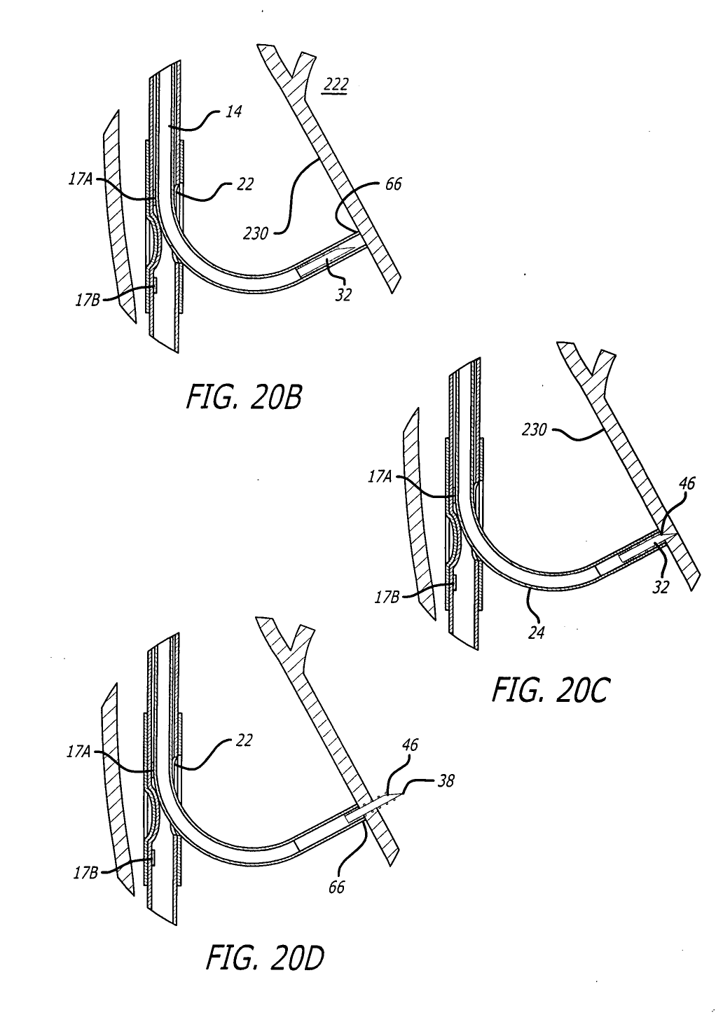

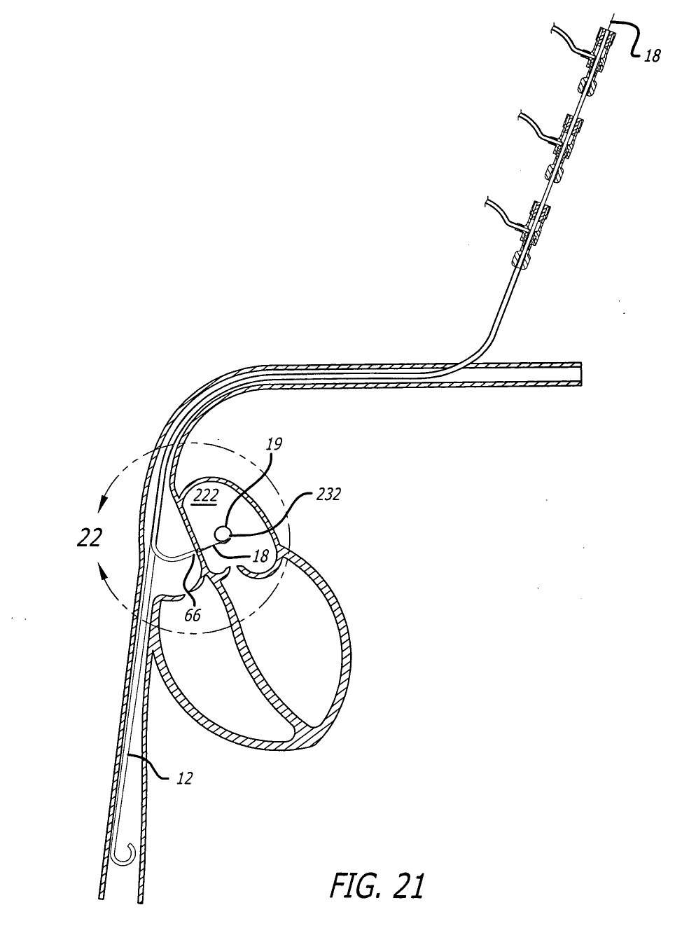

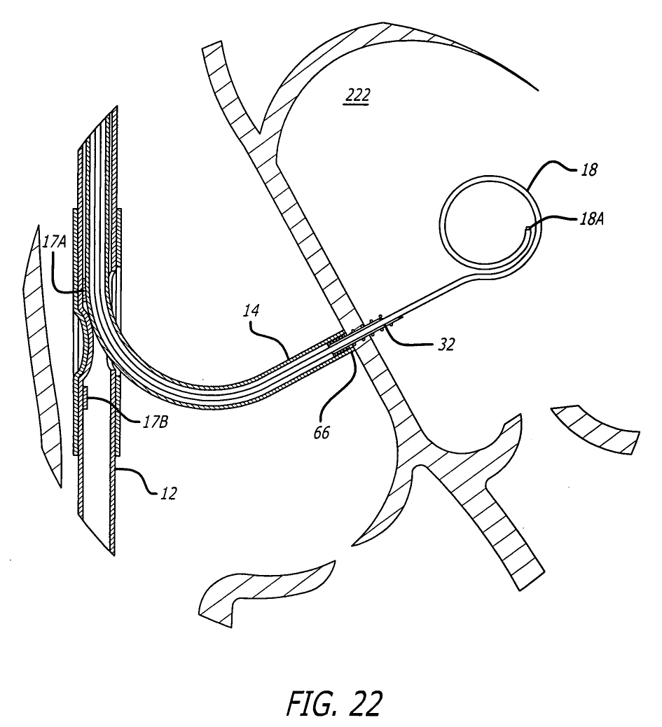

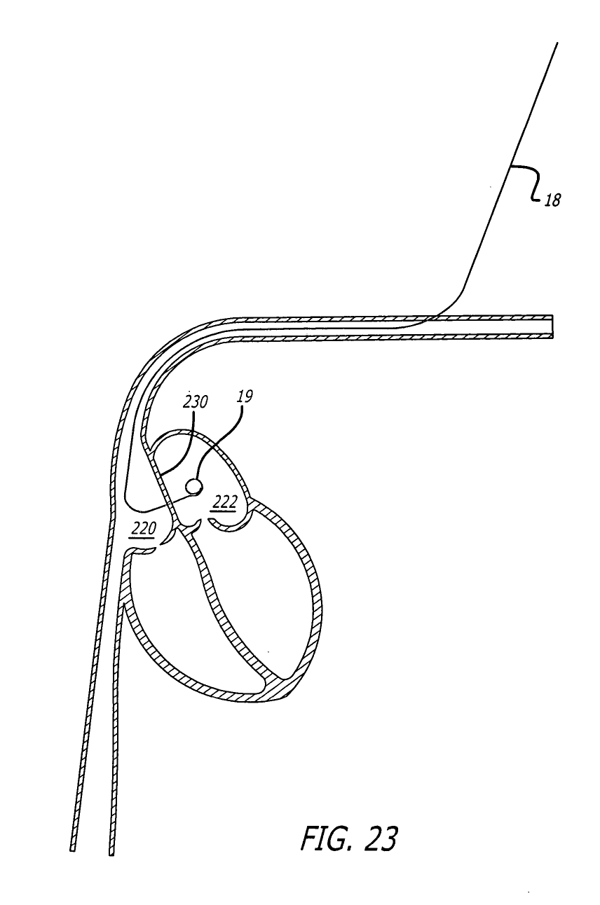

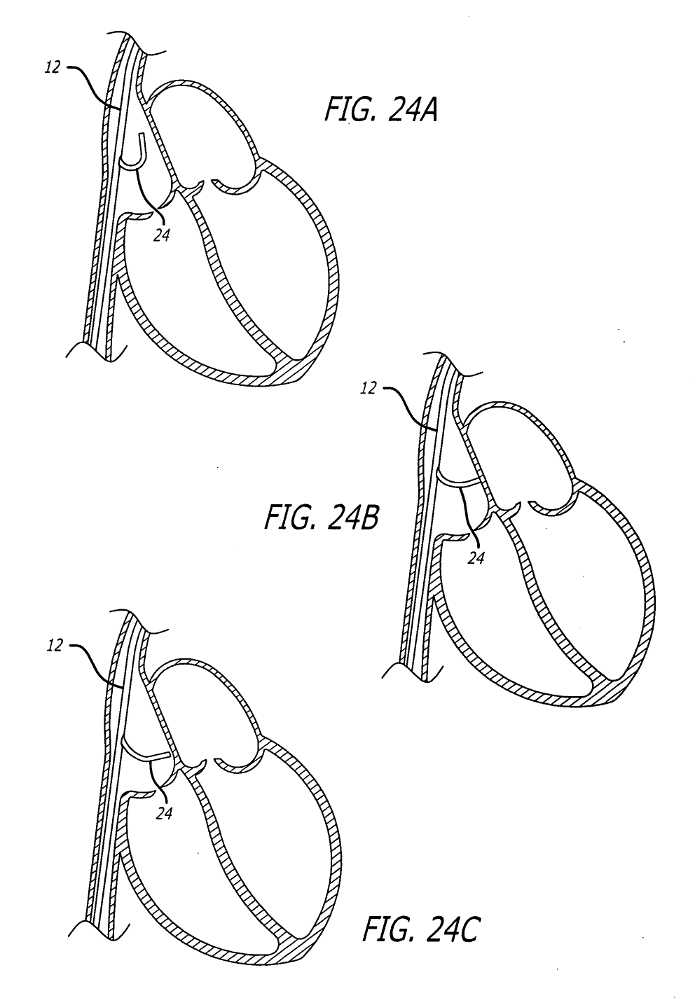

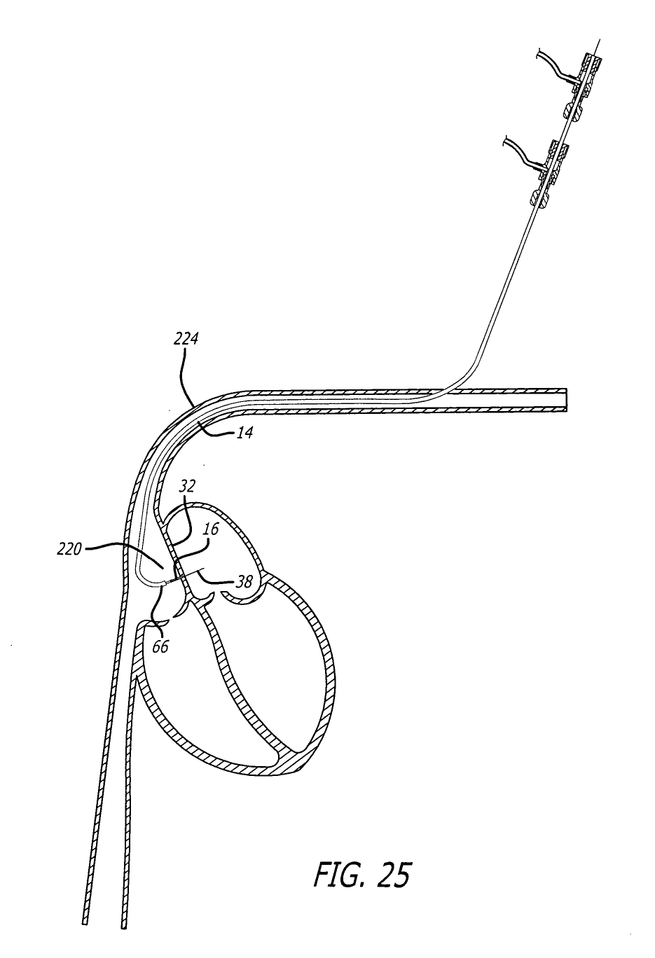

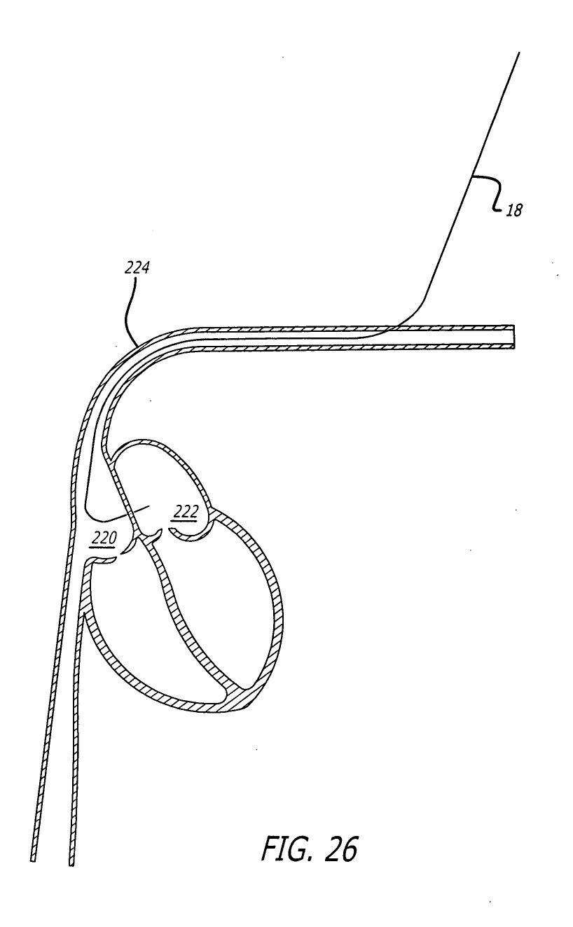

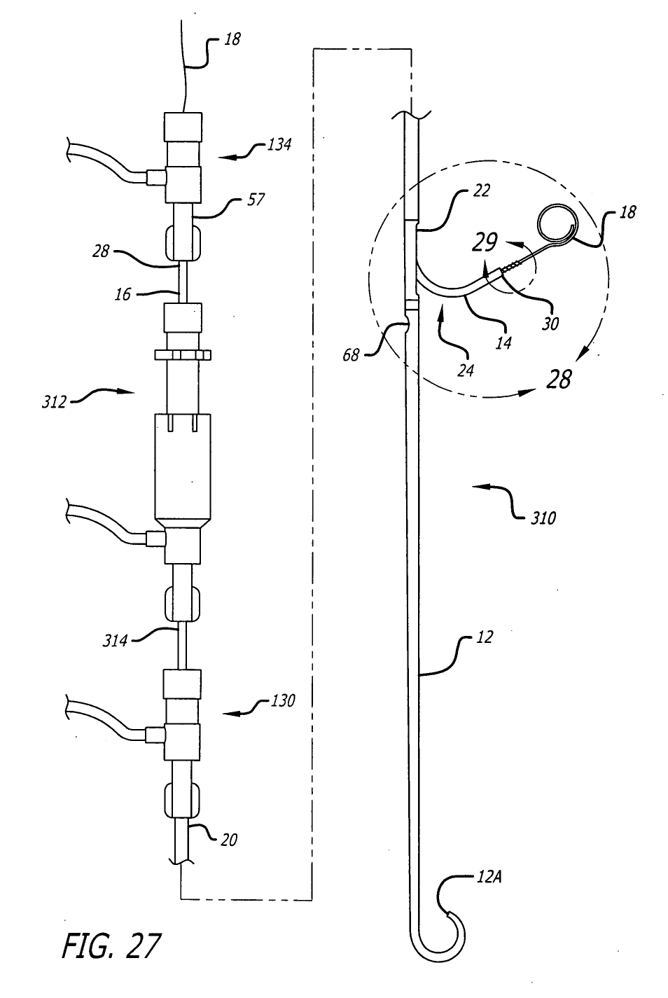

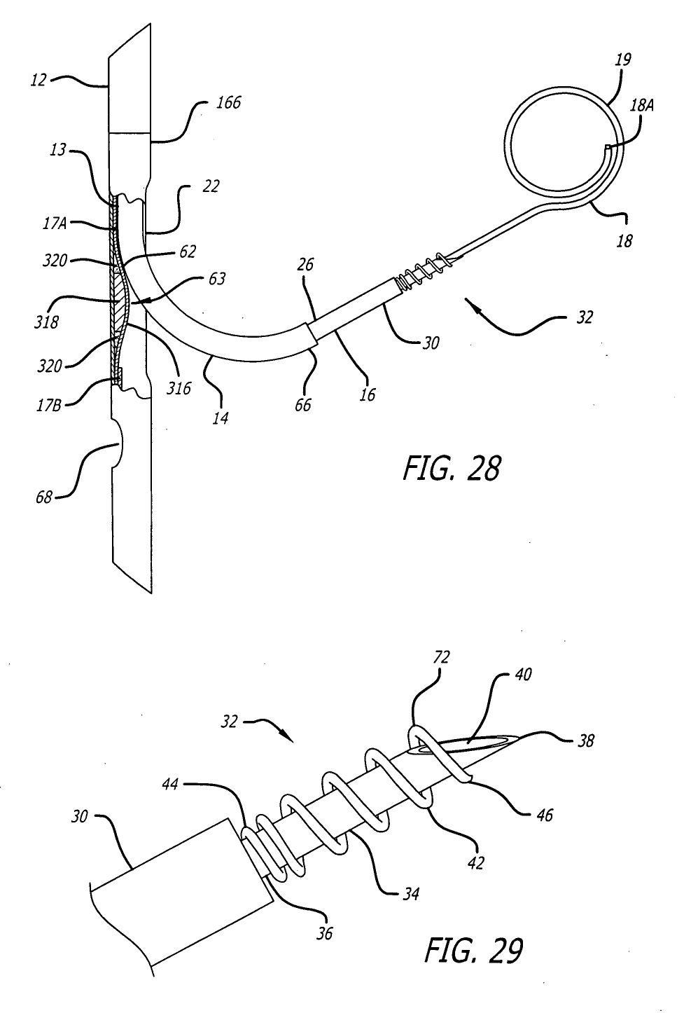

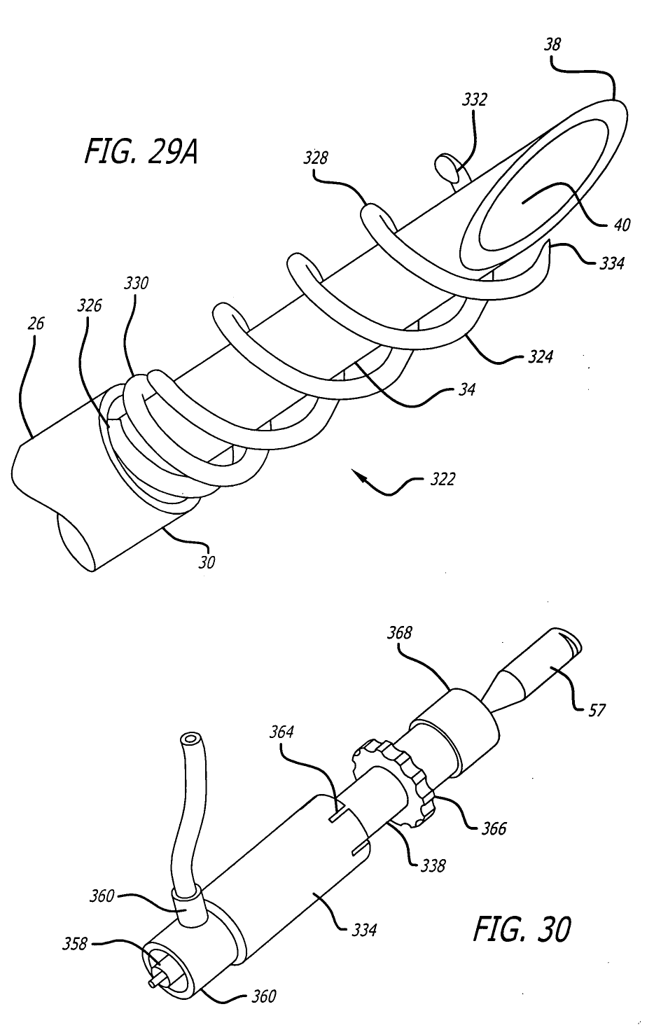

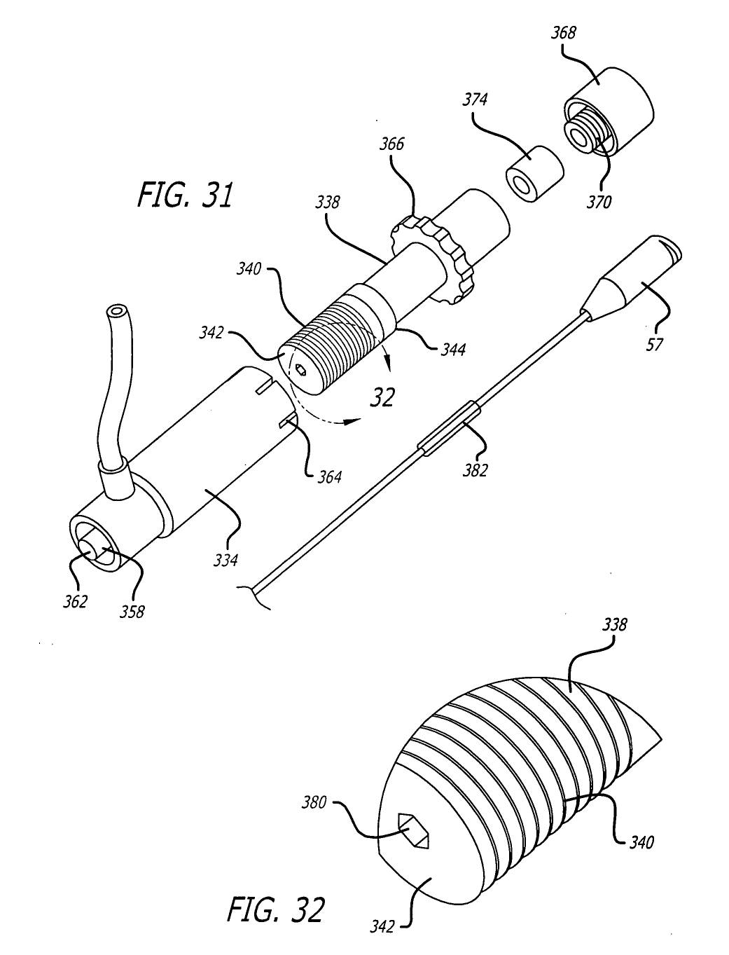

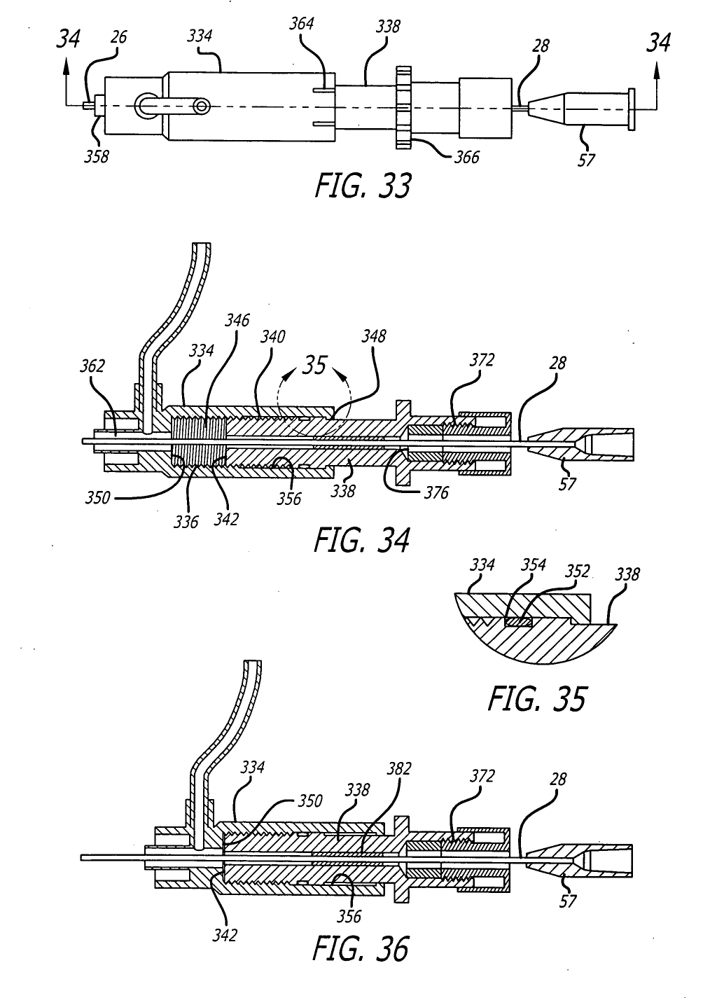

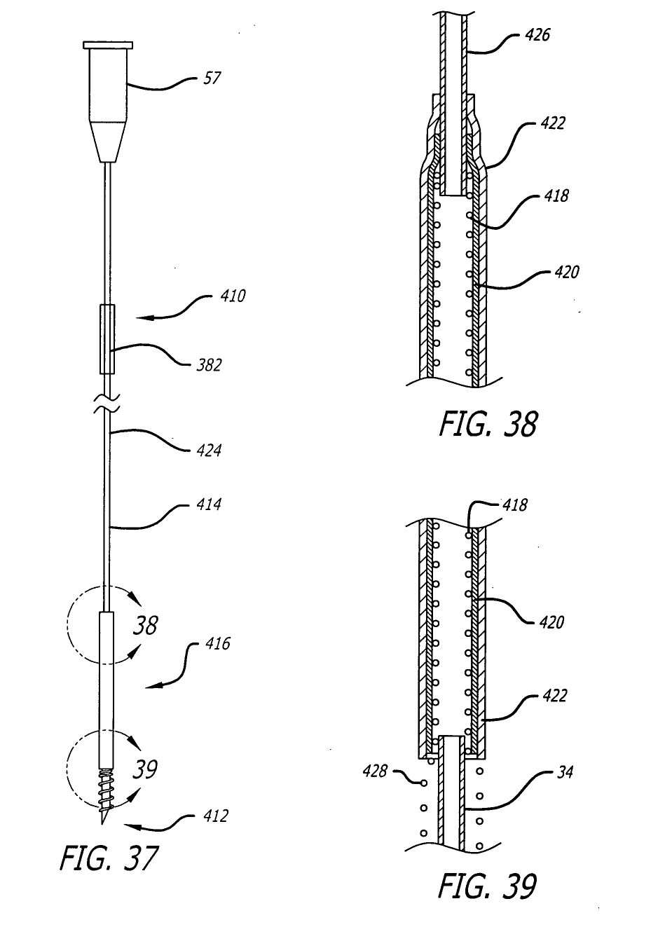

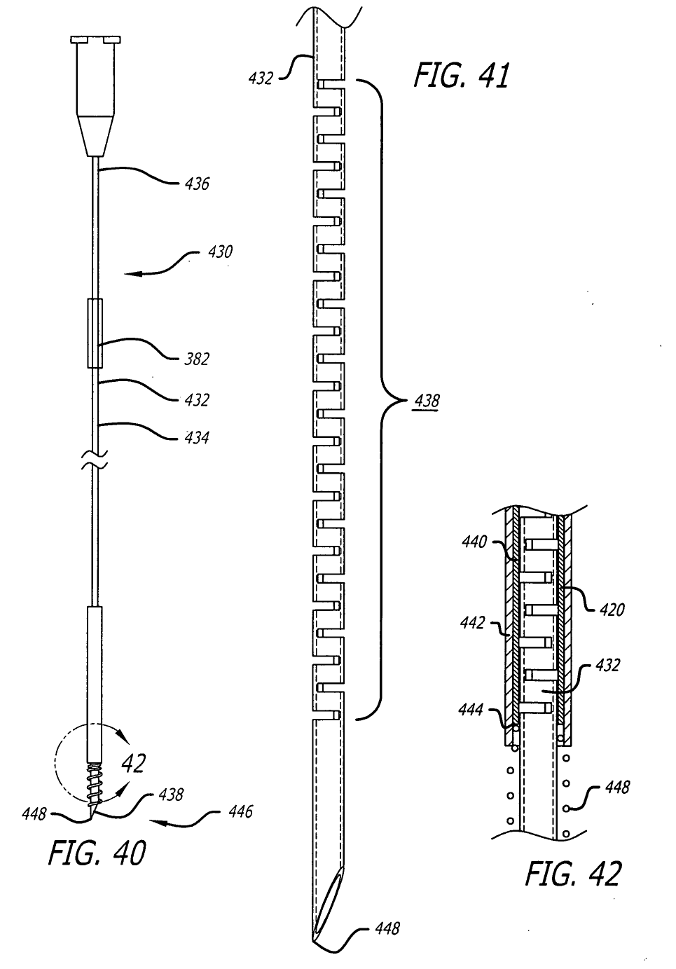

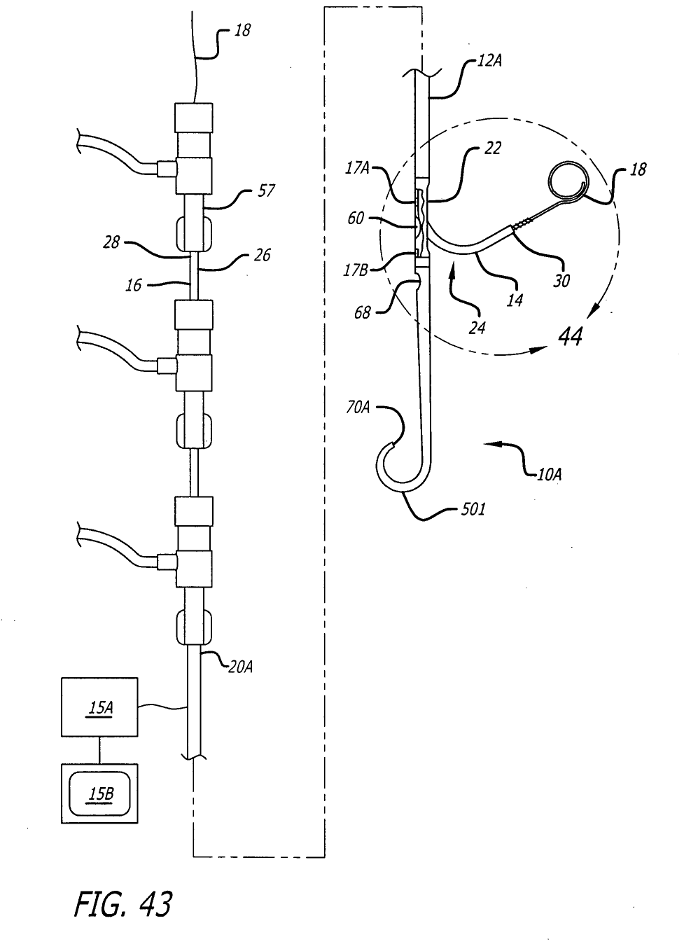

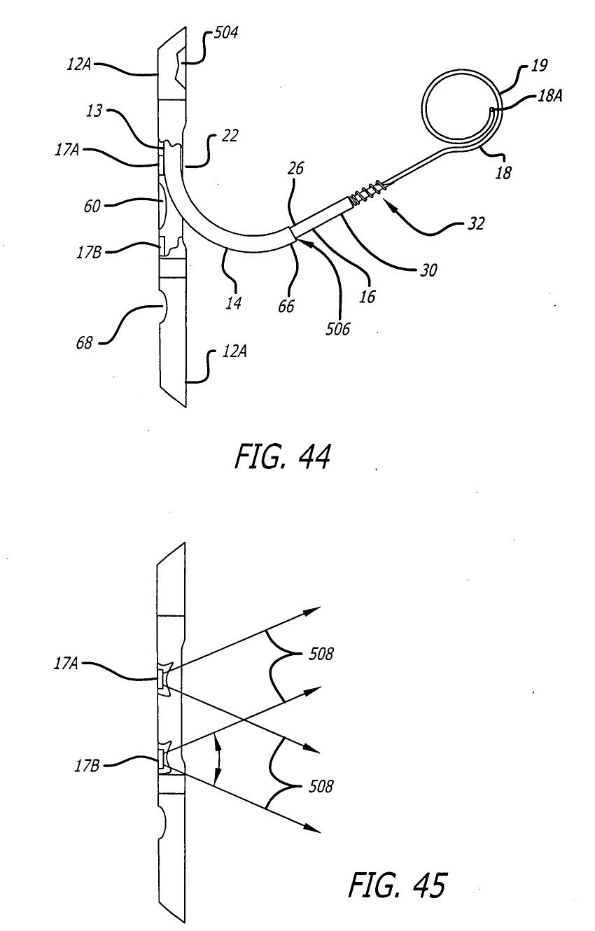

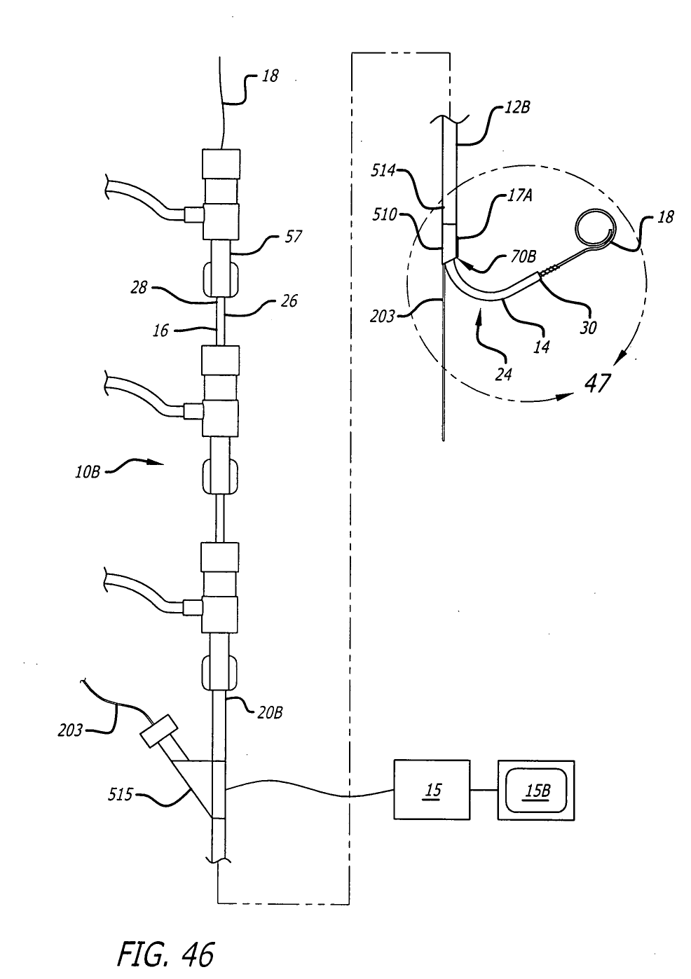

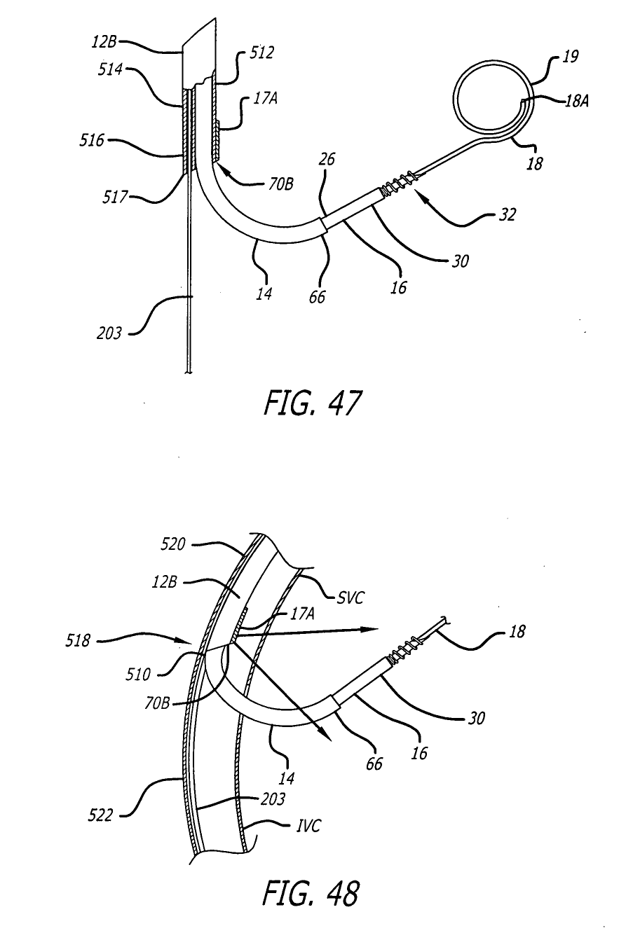

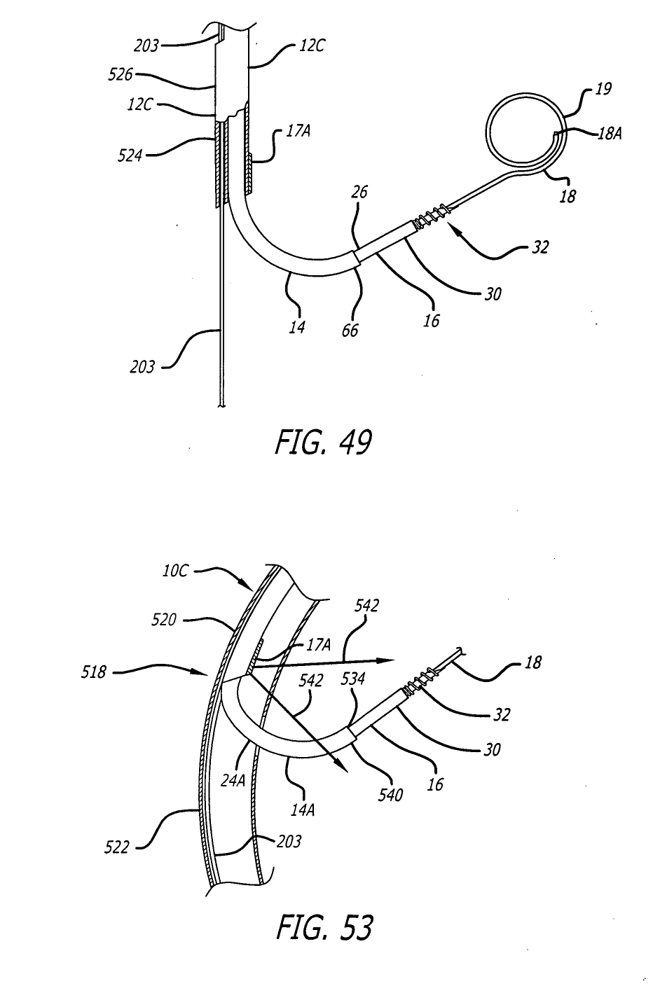

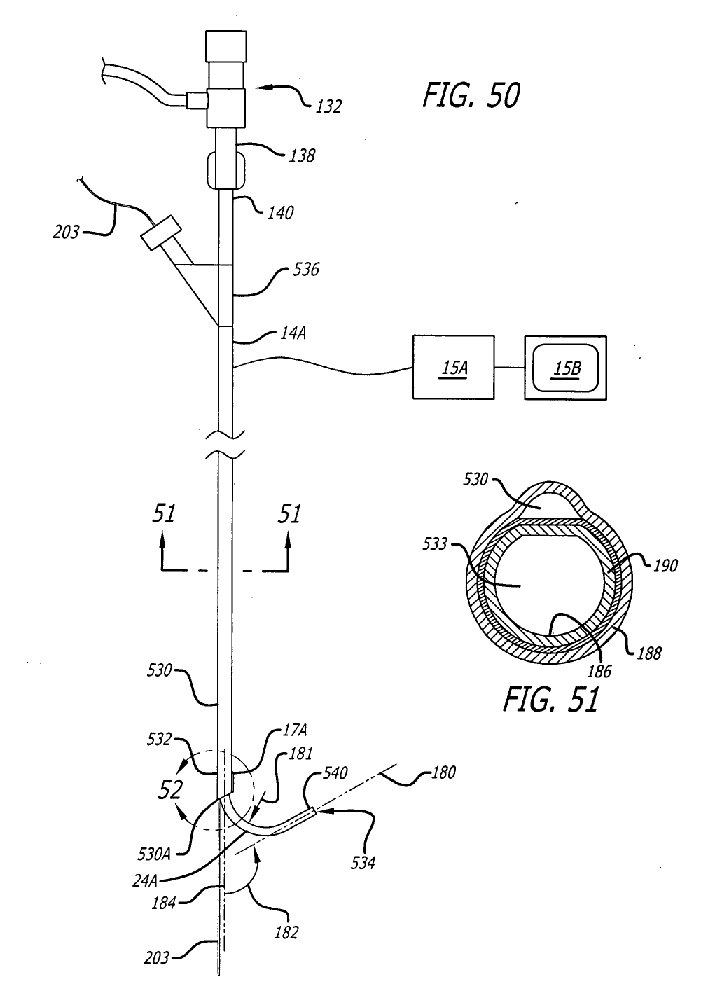

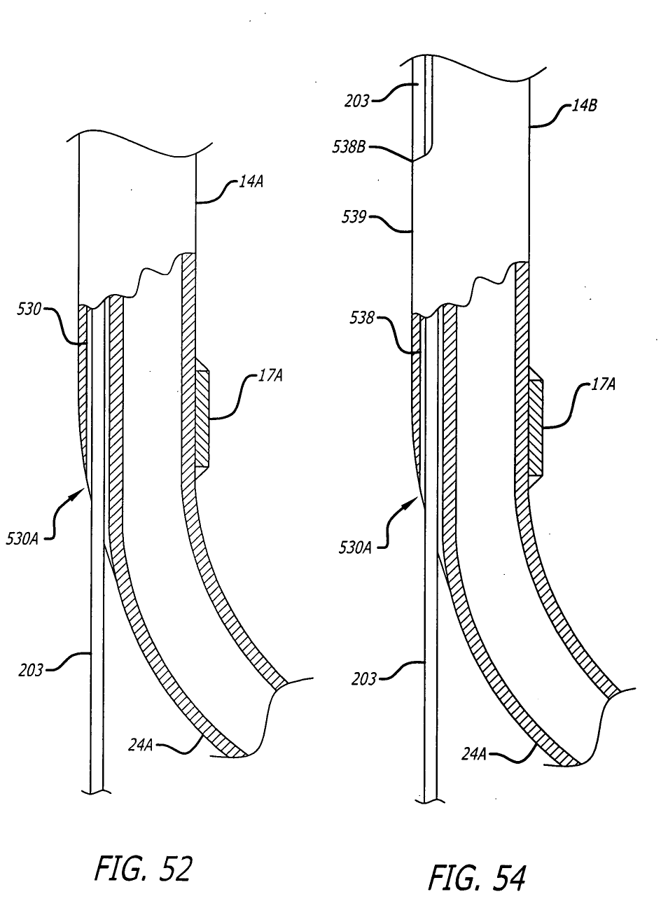

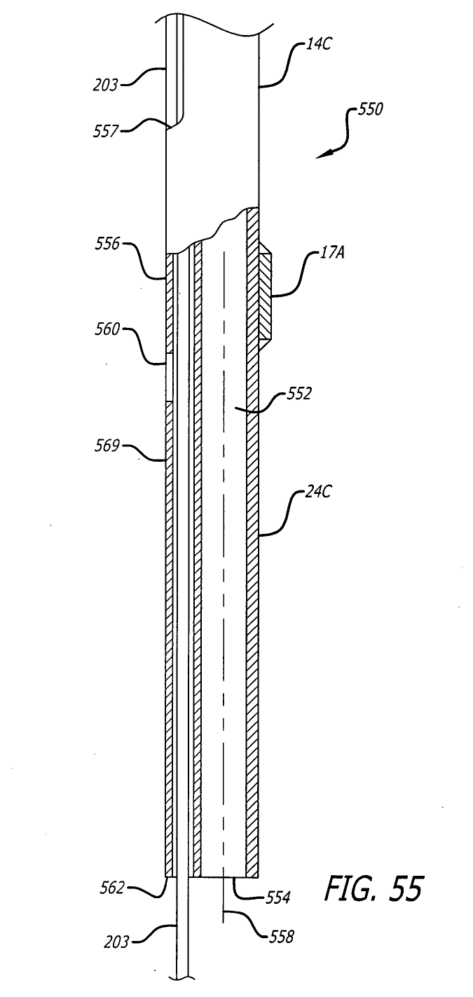

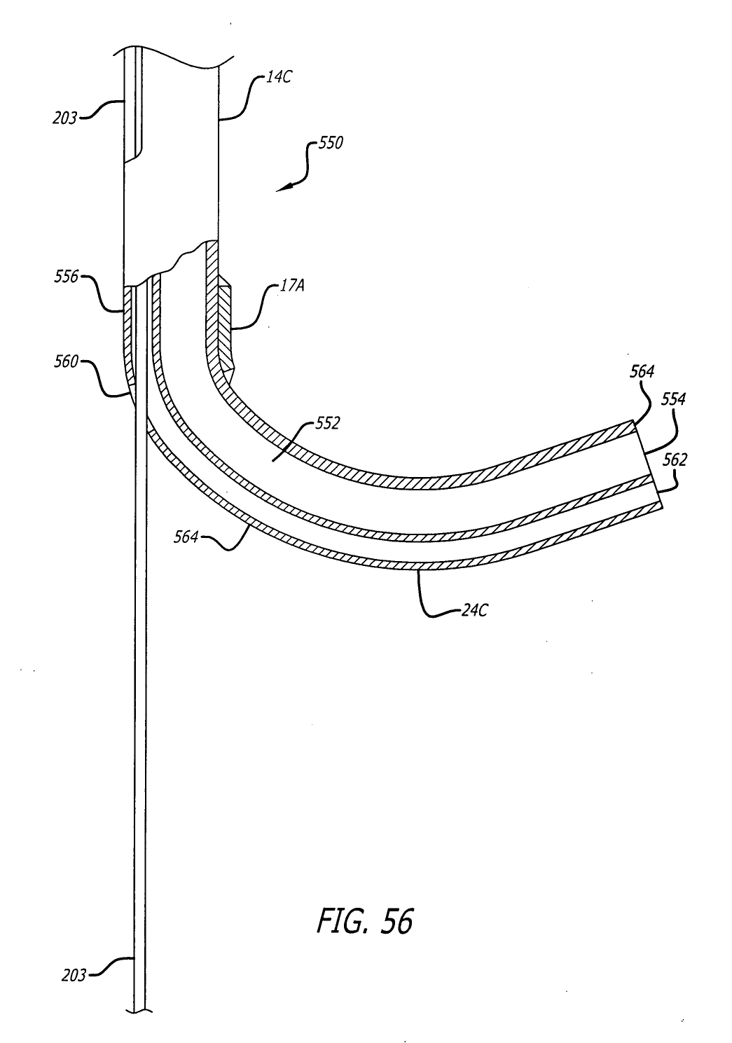

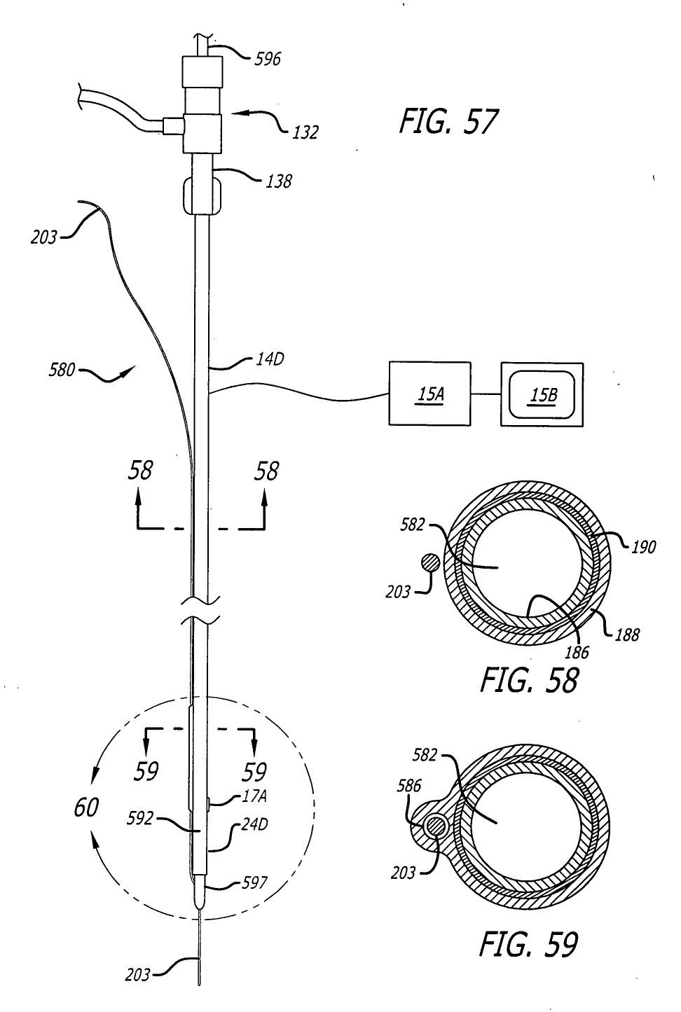

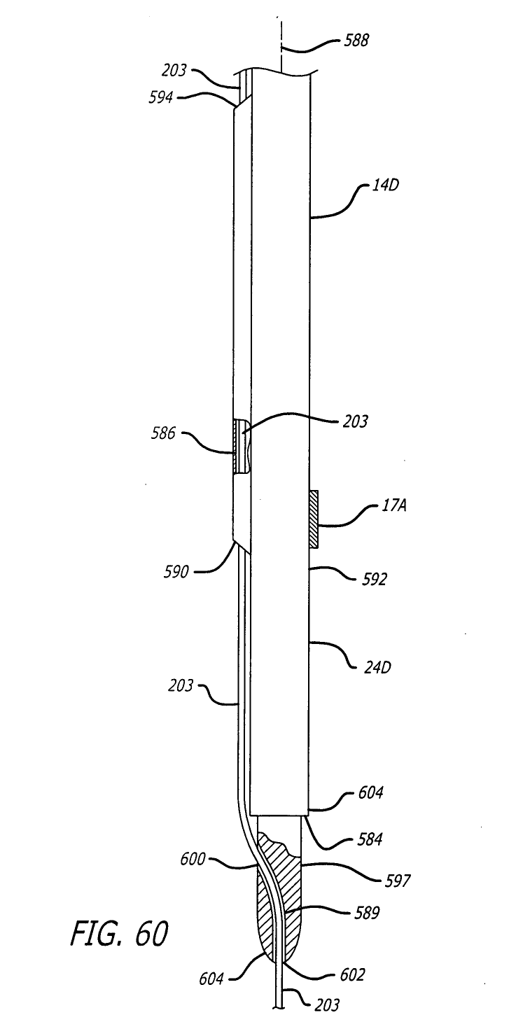

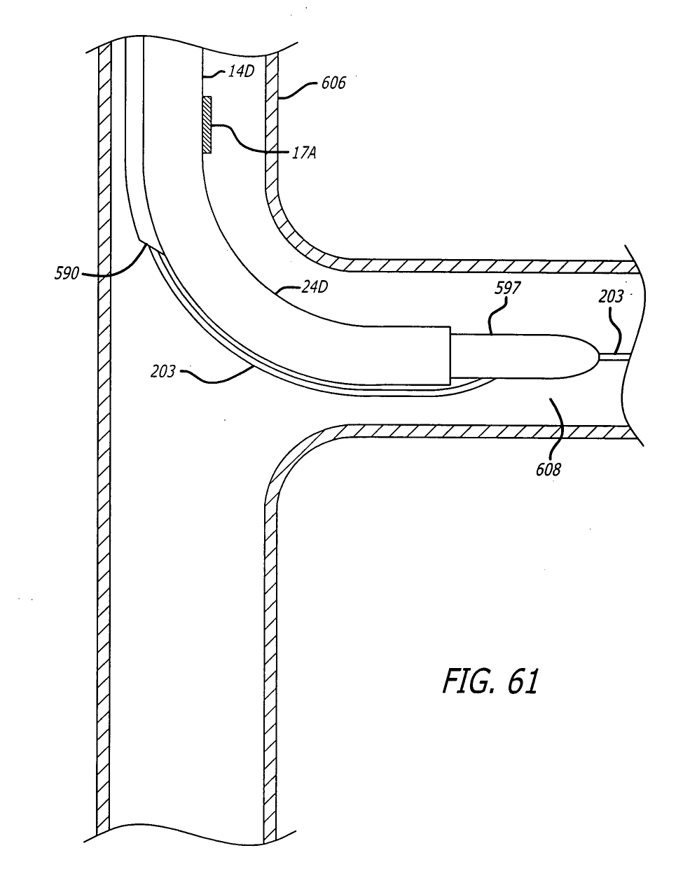

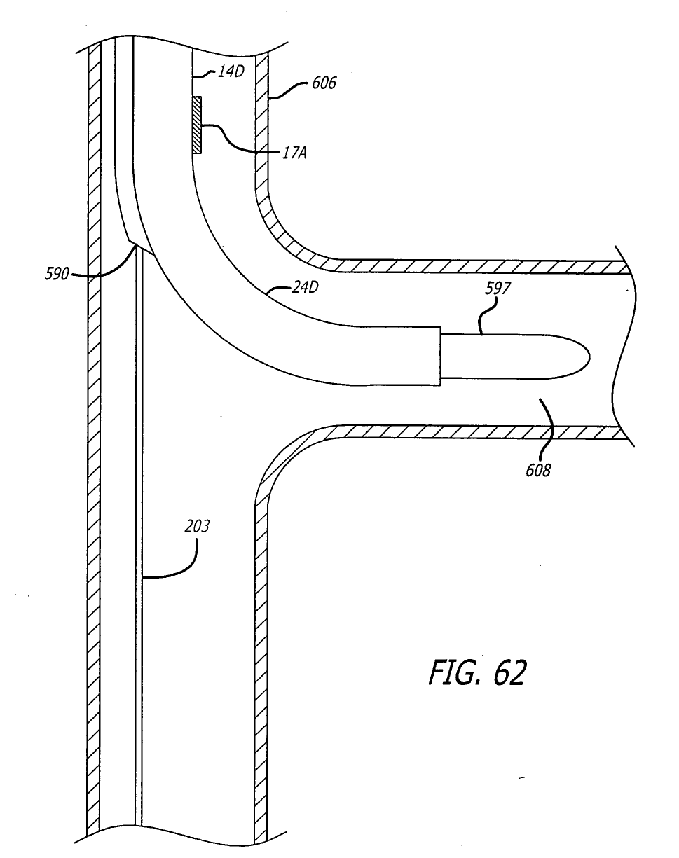

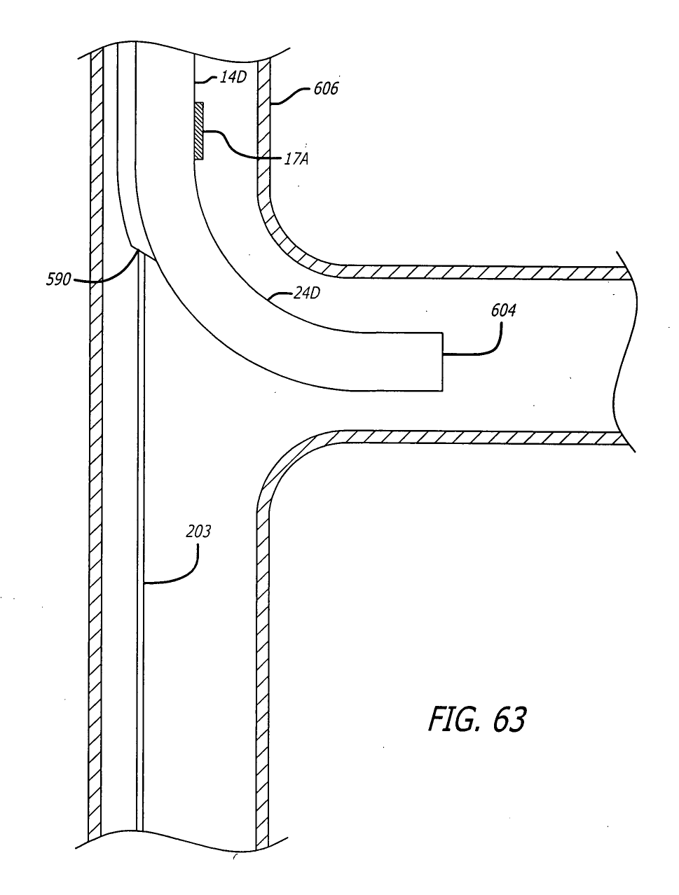

FIGS. 12-13 illustrate the embodiment of the guide catheter 14 of FIG. 1 showing the curved distal section 24 of the guide catheter 14 while the guide catheter 14 is in a relaxed state. The guide catheter 14 has a Luer fitting 138 secured to the proximal end 140 of the guide catheter 14. The curved distal section 24 may have an inner radius of curvature 181 of about 1 cm to about 4 cm. The discharge axis 180 of the guide catheter 14 may form an angle 182 with the nominal longitudinal axis 184 of the guide catheter 14 of about 90 degrees to about 270 degrees. Although many commercially available guide catheters 14 have a soft pliable distal tip for atraumatic advancement into a patient's vasculature, this may not be desirable in some instances for use with embodiments of the access systems discussed herein. More specifically, for some procedures, it may be necessary for the distal end of the guide catheter to have sufficient structural rigidity to maintain the round transverse cross section at the distal tip of the guide catheter so that the wall of the guide catheter at the distal tip does not collapse when pressed against target tissue. Such wall collapse or deformation could cause the tissue penetration device 16 to impinge on the wall of the guide catheter which may impede progress or the procedure generally. It may be desirable for the guide catheter to have a distal tip or distal section that has a wall structure with a nominal flexibility or shore hardness that is substantially similar to or the same as the nominal flexibility or shore hardness of the shaft proximal to the distal tip or section.

The guide catheter 14 may be made from a standard guide catheter construction that includes a plurality of polymer layers 186 and 188 reinforced by a braid 190. The nominal outer transverse dimension or diameter of the guide catheter 14 may be from about 0.04 inches to about 0.10 inches. The overall length of the guide catheter 14 should be sufficiently longer than the overall length of the stabilizer sheath 12 from its proximal end to the side port 22 including the length of its proximal adapter 130 and may be from about 40 cm to about 80 cm. The inner transverse dimension of the inner lumen 192 of the guide catheter 14 may be from about 0.03 inches to about 0.09 inches. It may desirable to select the flexibility of embodiments of the guide catheter 14, and particularly the curved distal section 24 of the guide catheter 14, and the flexibility of the tissue penetration member 32 such that the tissue penetration member 32 does not substantially straighten the curved distal section 24 of the guide catheter 14 when the tissue penetration device 16 is being advanced through the guide catheter 14. Otherwise, the maneuverability of the stabilizer sheath 12 and guide catheter 14 combination could be compromised for some procedures.

Suitable commercially available guide catheters 14 with distal curves such as a “hockey stick”, Amplatz type, XB type, RC type, as well as others, may be useful for procedures involving transseptal access from the right atrium of a patients heart and the left atrium of the patient's heart. Guide catheters 14 have a “torquable” shaft that permits rotation of the shaft. Once the distal tip of the guide catheter has exited the stabilizer sheath side port and extended more or less radially away from the stabilizer sheath, rotation of the guide catheter shaft causes its distal end to swing in an arc around the axis of the stabilizer sheath, providing for lateral adjustment of the guide catheter distal tip for precise positioning with respect to the septum. The variety of distal curve shapes described above and illustrated in FIGS. 12 and 13 are curves lying in a single plane. More complex distal curve shapes involving three dimensional space may also be useful. One such example commonly used in coronary angioplasty is the XB-LAD shape where the most distal portion of the curve is bent in another plane.

FIG. 14 illustrates an embodiment of an obturator sheath 196 configured to be disposed within the inner lumen 13 of the stabilizer sheath 12 and block the side port 22 of the stabilizer sheath 12 to prevent damage to tissue adjacent the stabilizer sheath 12 and stop blood flow into the stabilizer sheath 12 during insertion of the stabilizer sheath 12 in a patient's anatomy. The obturator sheath 196 has a substantially tubular configuration with proximal end 198, a distal end 200 and an inner lumen 202 extending through the obturator sheath 196 that is configured to accept the guidewire 18. The outer transverse dimension or cross sectional area of the obturator sheath 196 is configured to fill the gap between the side port 22 and the inner surface 164 of the inner lumen 13 of the stabilizer sheath 12 opposite the side port 22. Filling of the side port 22 by the obturator sheath 196 is illustrated in FIGS. 15 and 16 where the obturator sheath 196 is shown within the inner lumen 13 of the stabilizer sheath 12 passing over the abutment 60 of the side port 22 which forces a portion of it out of the side port 22 and extending distally within the inner lumen 13 of the stabilizer sheath 12 towards the distal end 156 of the stabilizer sheath 12. Guidewire 203 is shown disposed within the inner lumen 202 of the obturator sheath 196. FIG. 17 shows the distal end 200 of the obturator sheath 196 having a tapered configuration and showing the guidewire 203 disposed within and extending from the inner lumen 202 of the obturator sheath 196. Guidewire 203 may be a standard floppy tip guidewire used for interventional procedures. One embodiment of guidewire 203 is a floppy tip guidewire having a nominal outer diameter of about 0.036 inches to about 0.04 inches and a length of about 150 cm to about 200 cm. In another embodiment, guidewire 203 may be an exchange length guidewire having a length of about 250 cm to about 350 cm.

FIG. 17A illustrates an enlarged view in section of an embodiment of a stabilizer sheath 204 in a configuration that includes a side port 210. The guidewire 203 is shown extending through the inner lumen 206 of the stabilizer sheath and is maintained in a concentric arrangement with the longitudinal axis of the stabilizer sheath 204 by a sleeve portion 208. The sleeve portion 208 is also shaped within the side port 210 to act as a deflective surface 212.

Referring to FIG. 18, embodiments of the transmembrane access system 10 may be used for a transseptal access procedure from the right atrium 220 of a patient's heart to the left atrium 222. In one embodiment, this procedure begins by placing the guidewire 203 into the patient's superior vena cava 224 through a needle inserted at a vascular access point such as a subclavian vein near the shoulder or a jugular vein on the neck, similar to a standard technique for placing pacemaker leads. Thereafter, the distal port 12A of the stabilizer sheath 12 is fed over the proximal end of the guidewire 203 which extends from the patient's body. The guidewire 203 is then advanced proximally through the inner lumen of the stabilizer sheath 12 until the proximal end of the guidewire 203 extends from the proximal end of the stabilizer sheath 12 or exits the optional guidewire port 68. The distal end of the obturator sheath 196 is then tracked over the proximal end of the guidewire 203 into the stabilizer sheath 12 until the obturator sheath 196 seats and comes to a stop. The distal end or pigtail portion 154 of the stabilizer sheath 12, which is maintained in a substantially straightened configuration by the stiffness of the guidewire 203, is tapered and thinned, as shown in FIG. 10, so that is serves as a dilator during insertion through the skin and into the vein. The stabilizer sheath 12 and obturator sheath 196 are then advanced distally together over the guidewire 18 into the superior vena cava of the patient. The stabilizer sheath 12 is advanced distally until the distal end 156 of the stabilizer sheath 12 is disposed within the inferior vena cava 226 and the side port 22 is within or adjacent the right atrium 220 of the patient as shown in FIG. 18. Thereafter, the guidewire 203 and obturator sheath 196 are withdrawn from the inner lumen 13 of the stabilizer sheath 12, allowing the distal portion 154 of the stabilizer sheath 12 to assume its relaxed pigtail configuration, and allowing the guide catheter 14 and elongate tissue penetration device 16 to be advanced through the proximal adapter 130 of the stabilizer sheath 12 and through the inner lumen 13 of the stabilizer sheath 12 towards the side port 22.

This procedure may also be initiated from an access point from the patient's inferior vena cava 226 beginning by placing a guidewire into the patient's inferior vena cava through a needle inserted at a vascular access point such as a femoral vein near the groin, well known to skilled artisans. In the same manner described above for the superior vena cava approach, the proximal end of the guidewire 203 is backloaded into the stabilizer sheath 12, the obturator sheath 196 is advanced over the guide wire 203 into the stabilizer sheath until its distal end seats at the side port. The stabilizer sheath 12 and obturator sheath 196 are then inserted together over the guidewire 18 through the skin and into the vein, and then advanced distally together over the guidewire 18 through the inferior vena cava 226 of the patient until the distal end 156 of the stabilizer sheath 12 is disposed within the superior vena cava 224 and the side port 22 is disposed within or adjacent to the right atrium 220 of the patient.

FIG. 18 illustrates the stabilizer sheath 12 positioned through a chamber in the form of the right atrium 220 with the side port 22 of the stabilizer sheath 12 positioned in the chamber 220. The side port section 162 of the stabilizer sheath 12 spans the chamber 220 between a first orifice which is the opening of the superior vena cava 224 into the right atrium 220 and a second orifice which is the opening of the inferior vena cava 226 into the right atrium 220. The superior vena cava 224 and inferior vena cava 226 form two tubular structures extending from opposite sides of the chamber 220 which provide lateral support to the side port section 162 of the stabilizer sheath 12. The lateral support of the tubular structures 224 and 226 and respective orifices adjacent the side port section 162 of the stabilizer sheath 12 provides a stable platform from which the guide catheter 14 may be extended for performing procedures within the chamber 220. The lateral stability of the side port section provides back up support for the guide catheter 14 to be pushed or extended distally from the side port 22 and exert distal force against structures within the chamber 220 while maintaining positional control over the distal end of the guide catheter 14. This configuration provides the necessary stability and support for performing procedures within the chamber and beyond regardless of the size and shape of the chamber 220 which can vary greatly due to dilation or distortion caused by disease or other factors. This configuration contemplates lateral stabilization of the side port section 162 as a result of confinement of the stabilizer sheath portions adjacent the side port section 162 in respective tubular structures. However, a similar result could be achieved with a stabilizer sheath embodiment similar to stabilizer sheath 12 having a short distal section or no distal section extending distally from the side port section 22. For such an embodiment, stabilization of the side port section could be achieved by lateral or transverse confinement of a section of the stabilizer sheath proximal of the side port section in a tubular structure and lateral confinement of a guidewire or other stabilizer member extending distally from the inner lumen of the stabilizer sheath in a similar tubular structure.

Although the embodiment of the method illustrated in FIG. 18 is directed to a transseptal cardiac procedure, the stabilizer sheath 12 and guide catheter 14 arrangement could also be used for a variety of other indications depending on the shape of the guide or access catheter 14 used in conjunction with the stabilizer sheath 12. If the optional peel away tear line 167 is incorporated into the stabilizer sheath 12 and reinforcement member 166, applicable procedures could include deployment of pacing leads, e.g. into the coronary sinus for cardiac resynchronization therapy or biventricular pacing, placement of a prosthesis for mitral valve repair annulus repair as well as others. The usefulness of the various embodiments is not limited to the venous circulation: many other anatomical areas that may be accessed by catheter are accessed by making use of the added support and control provided by the side port stabilizer sheath and shaped guide catheter. A few additional examples include, but are not limited to: the coronary arteries via a stabilizer sheath with its side port very near its distal end as described above, or with a distal section designed with a “pig-tail” designed to pass through the aortic valve and into the left ventricle; retrograde access to the mitral valve and left atrium via the left ventricle using a stabilizer sheath with a short pigtail distal segment as described for the coronary arteries, but with its side port located more distally so that it may be placed in the mid left ventricle; and other areas, such as the renal arteries, where acute angles limit the control provided by conventional catheters.

Once in place, the stabilizer sheath 12 can be rotated within the chamber 220 to direct the side port 22 to any lateral direction within the chamber 220. The rotational freedom of the stabilizer sheath 12 within the chamber 220 can be combined with axial translation of the stabilizer sheath 12, in either a distal direction or proximal direction, to allow the side port 22 of the stabilizer sheath to be directed to most any portion of the chamber 220. When these features of the stabilizer sheath 12 are combined with a guide catheter 14 having a curved distal section extending from the side port 22, a subselective catheter configuration results whereby rotation, axial translation or both can be applied to the stabilizer sheath 12 and guide catheter 14 in order to access any portion of the interior of the chamber 220 from a variety of approach angles. The selectivity of the configuration is also discussed below with regard to FIGS. 24A-24C.

During insertion of the guide catheter 14 and elongate tissue penetration device 16, the tissue penetration member 32 of the elongate tissue penetration device 16 is disposed within the inner lumen of the distal portion 24 of the guide catheter 14 to prevent contact of the tissue penetration member 32 with the inner lumen 13 of the stabilizer sheath 12 during advancement. FIG. 19 shows an enlarged elevational view of the side port section 162 of the stabilizer sheath 12 with the distal end 66 of the guide catheter 14 and the distal end 38 of the tissue penetration device 32, disposed within the distal end 66 of the guide catheter, being advanced distally through the inner lumen 13 of the stabilizer sheath 12. As the guide catheter 14 and elongate tissue penetration device 16 continue to be advanced distally, the distal end 66 of the guide catheter 14 impinges on the deflective surface 62 of the abutment 60 opposite the side port 22. The distal section 24 of the guide catheter 14 then emerges from the side port 22 and begins to assume the pre-shaped configuration of the guide catheter 14. The pre-shaped configuration of the distal section 24 curves the distal end 66 of the guide catheter 14 away from the longitudinal axis 160 of the side port section 162 of the stabilizer sheath 12 and extends the distal end 66 of the guide catheter 14 radially from the side port 22 and against the septal wall 230 as shown in FIG. 20.

The distal end 66 of the guide catheter 44 is advanced until it is positioned adjacent a desired area of the patient's septum 230 for transseptal access. In this arrangement, the orientation and angle of penetration or approach of the distal end 66 of the guide catheter 14 and elongate tissue penetration device 16 can be manipulated by axially advancing and retracting the stabilizer sheath 12 in combination with advancing and retracting the guide catheter 14 from the side port 22 of the stabilizer sheath 12. This procedure allows for access to a substantial portion of the patient's right atrial surface and allows for transmembrane procedures in areas other than the septum 230, and more specifically, the fossa ovalis of the septum 230. At this stage of the procedure, it may be desirable to determine the distance from the distal tip of the tissue penetration device 16 or the tissue penetration member 32 to the tissue adjacent the tissue penetration device 16. It may also be desirable to determine other characteristics of the tissue adjacent the distal tip of the tissue penetration device 16 or tissue penetration member 32, such as the thickness, density or electrical characteristics of the tissue. In order to accomplish this, a guidewire 18 or other elongate member having properties similar to or the same as those of a guidewire 18, may include a sensor 18A on a distal end thereof. Such a sensor 18A, as shown in FIG. 22, may include an ultrasound transducer, an electrode, such as a pacing electrode, or the like. If sensor 18A includes an ultrasound transducer, properties such as tissue distance, thickness, density and the like may be determined prior to, during or after activation of the tissue penetration device 16 or tissue penetration member 32. If sensor 18A includes an electrode, electrical activity of the tissue may be monitored prior to, during or after activation of the tissue penetration device. Such a sensor 18A may be used with any of the embodiments disclosed herein for the same or similar purposes.

During a tissue penetration process by the tissue penetration member 32 or other suitable tissue penetration member, it may also be desirable to provide mechanical support or shaping characteristics to the distal portions of the guide catheter 14 and tissue penetration device 16. In some embodiments, an elongate member in the form of a stylet 18B having a shaped distal section 18C may be used within the inner lumen 58 of the tissue penetration device 16. Such a stylet is shown in FIG. 20A and includes an optional sensor 18A which may be used for the purposes discussed above, as well as others. Stylet 18B may be used with any of the systems discussed herein and may have materials, dimensions and features which are the same as or similar to those of guidewire 18. Stylet 18B may be used to provide column strength or shape reinforcement to the distal section 24 of the guide catheter 14 or the distal portion of the tissue penetration device 16, including the tissue penetration member 32.

FIGS. 20B-20D illustrate a tissue penetration sequence by the tissue penetration member 32 through the septum of the patient. FIG. 20B shows an enlarged view of the distal end 66 of the guide catheter 14 disposed adjacent target tissue of the septal wall 230 with the tissue penetration member 32 withdrawn into the distal portion 24 of the guide catheter 14. FIG. 20C shows the tissue penetration member 32 during activation with the rotation of the tissue penetration member 32 causing the sharpened tip 38 of the tubular needle 34 to cut into and penetrate the septal wall 230 and allow advancement of the tubular needle 34. The sharpened distal end 46 of the helical tissue penetration member 42 penetrates tissue helically due to the rotational motive force of the tissue penetration member 32. The helical tissue penetration member 42 may also help pull the tubular needle 34 into the target tissue 230 as it advances. FIG. 20D shows the distal tip 38 of the tubular needle 34 having penetrated the septal wall 230 and in communication with the left atrium 222.

Once the distal end 66 of the guide catheter 14 is disposed adjacent a desired area of target tissue, the tissue penetration member 32 of the elongate tissue penetration device 16 is advanced distally until contact is made between the sharpened tip 38 of the tubular needle 34 and the target tissue. The tissue penetration member 32 is then activated by rotation, axial movement or both, of the torquable shaft 26 of the elongate tissue penetration device 16. As the tissue penetration member 32 is rotated, the sharpened tip 38 of the tubular needle 34 begins to cut into the target tissue 230 and the sharpened distal end 46 of the helical tissue penetration member 42 begins to penetrate into target tissue in a helical motion. As the sharpened tip 38 of the tubular needle 34 penetrates the target tissue, the tubular needle 34 provides lateral stabilization to the tissue penetration member 32 and particularly the helical tissue penetration member 42 during penetration. The rotation continues until the distal tip 38 of the tubular needle 34 perforates the septal membrane 230 and gains access to the left atrium 222 as shown in FIG. 21 and in an enlarged view in FIG. 22. Confirmation of access to the left atrium 222 can be achieved visually by injection of contrast media under fluoroscopy through the inner lumen 58 of the elongate tissue penetration device 16 from the side port 150 of the proximal adapter 134 of the elongate tissue penetration device 16. Confirmation can also be carried out by monitoring the internal pressure within the inner lumen of the elongate tissue penetration device 16 at the side port 150 of the proximal adapter 134 of the elongate tissue penetration device 16 during the rotation of the tissue penetration member 32.

Once the tubular needle 34 has perforated the septal wall 230 and gained access to the left atrium 222, the guidewire 18 can then be advanced through the inner lumen 58 of the elongate tissue penetration device 16 and into the left atrium 222 opposite the membrane of the septum 230 of the right atrium 220. An embodiment of a guidewire 18 that may be useful for this type of transseptal procedure may be an Inoue wire, manufactured by TORAY Company, of JAPAN. This type of guidewire 18 may have a length of about 140 cm to about 180 cm, and a nominal transverse outer dimension of about 0.6 mm to about 0.8 mm. The distal section 19 of this guidewire 18 embodiment may be configured to be self coiling which produces an anchoring structure in the left atrium 222 after emerging from the distal port 40 of the tubular needle 34. The anchoring structure helps prevent inadvertent withdrawal of the guidewire 18 during removal of the guide catheter 14 and elongate tissue penetration device 16 once access across the tissue membrane 230 has been achieved. The guidewire 18 is shown in position across the septal wall 230 in FIGS. 22 and 23 with the distal end 232 of the guidewire 18 in position in the left atrium 222 after the stabilizer sheath 12, guide catheter 14 and elongate tissue penetration device 16 have been withdrawn proximally over the guidewire 18.

FIGS. 24A-24C illustrate how the orientation of the distal section 24 of the guide catheter 14 can be controlled by advancing and retracting the guide catheter 14 within the side port of the stabilizer sheath 12, and axial movement of the stabilizer sheath 12 relative to the right atrium 220. This arrangement and orientation technique can also be adapted to accessing other portions of the patient's anatomy. The tip angle and radius of curvature of the guide catheter 14 can be also be manipulated by pushing it against the surrounding anatomy.

FIGS. 25 and 26 illustrate a method of transmembrane access across a patient's septal wall 230 by using an embodiment of a guide catheter 14 and elongate tissue penetration device 16 having a tissue penetration member 32 activated by rotation without the use of a stabilizer sheath 12. In this embodiment of use, the guide catheter 14 is advanced distally through the superior vena cava 224 of a patient and into the right atrium 220 over a guidewire 18. The guide catheter 14 is maneuvered until the distal end 66 of the guide catheter 14 is oriented towards a target area of the septum 230. The elongate tissue penetration device is then advanced distally from the distal end of the guide catheter until the sharpened distal tip 38 of the tissue penetration member 32 is in contact with the target tissue. The tissue penetration member 32 is then activated with rotational movement which causes the sharpened distal tip 38 of the tubular needle 34 and sharpened tip 46 of the helical tissue penetration member 42 of the tissue penetration member 32 to advance into the target tissue. Once the distal end 38 of the tubular needle 34 has penetrated the septum 230, as confirmed by either of the methods discussed above, the guidewire 18 is advanced distally through the inner lumen of the elongate tissue penetration device 16 and out of the distal end 40 of the tubular needle 34 and into the left atrial space 222. Thereafter, the elongate tissue penetration device 16 may be withdrawn proximally leaving the guidewire 18 in place across the septum 230 as shown in FIG. 26.

FIG. 27 is an elevational view of another embodiment of a transmembrane access system 310 that includes a proximal activation modulator 312 secured to a proximal end 314 of the guide catheter 14. Embodiments of the proximal activation modulator 312 may be configured to apply axial force while simultaneously advancing the device at an appropriate rate on the torquable shaft, limit the number of rotations of the proximal end of the torquable shaft 26 which controls the axial penetration of the tissue penetration member 32, or both of these functions as well as others. The system 310 shown in FIG. 27 includes a stabilizer sheath 12, a guide catheter 14, an elongate tissue penetration device 16 and a guidewire 18 disposed within an inner lumen of the elongate tissue penetration device 16. The stabilizer sheath 12 has a tubular configuration with an inner lumen 13 extending from a proximal end 20 of the stabilizer sheath 12 to a side port 22 disposed in the sheath 12. In one embodiment, the inner lumen 13 extends to the distal port 12A of the stabilizer sheath 12, and is open to one or more side ports 22 at one or more locations between the proximal end and distal end of the stabilizer sheath 12. The guide catheter 14 has a tubular configuration and is configured with an outer surface profile which allows the guide catheter 14 to be moved axially within the inner lumen of the stabilizer sheath 12. The guide catheter 14 has a shaped distal section 24 with a curved configuration in a relaxed state which can be straightened and advanced through the inner lumen of the stabilizer sheath 12 until it exits the side port 22 of the stabilizer sheath 12 as shown in more detail in FIG. 28.

The elongate tissue penetration device 16 includes a tubular flexible, torquable shaft 26 having a proximal end 28, shown in FIG. 27, and a distal end 30. The distal end 30 of the torquable shaft 26 is secured to a tissue penetration member 32, shown in more detail in FIG. 29, which is configured to penetrate tissue upon activation by rotation of the tissue penetration member 32. The tissue penetration member 32 has a tubular needle 34 with a proximal end 36, a sharpened distal end 38 and an inner lumen 40 that extends longitudinally through the tubular needle 34. A helical tissue penetration member 42 has a proximal end 44 and a sharpened distal end 46 and is disposed about the tubular needle 34. The helical tissue penetration member 42 has an inner diameter which is larger than an outer diameter of the tubular needle 34 so as to leave a gap between the tubular needle 34 and the helical tissue penetration member 42 for the portion of the helical tissue penetration 42 that extends distally from the distal end 30 of the torquable shaft 26.

Referring to FIG. 28, an abutment 316 having a radially deflective surface 62 is disposed within the inner lumen 13 of the stabilizer sheath 12 opposite the side port 22 of the sheath 12. In the embodiment shown, the apex 63 of the abutment 316 is disposed towards the distal end of the side port 22 which disposes the deflective surface 62 in a position which is longitudinally centered, or substantially longitudinally centered, in the side port 22. This configuration allows for reliable egress of the distal end 66 of the guide catheter 14 from the side port 22 after lateral deflection of the guide catheter 14 by the deflective surface 62. The deflective surface 62 of the abutment 316 serves to deflect the distal end 66 of the guide catheter 14 from a nominal axial path and out of the side port 22 during advancement of the guide catheter 14 through the inner lumen 13 of the stabilizer sheath 12. The abutment 316 is formed from a section of solid dowel pin 318 disposed between an inner surface of the tubular reinforcement member 166 and an outer surface of the stabilizer sheath 12. The solid dowel pin 318 is secured in place by epoxy potting material 320, but may be secured in place by a variety of other methods including mechanical capture or solvent bonding.

FIG. 29A is an enlarged view of an alternative embodiment of a tissue penetration member 322 having two helical tissue penetration members. The tissue penetration member has a tubular needle 34 secured to a distal end 30 of the torquable shaft 26. A first helical tissue penetration member 324 has a proximal end 326 secured to the tubular needle 34 and distal end 30 of the torquable shaft 26. A second helical tissue penetration member 328 has a proximal end 330 secured to the tubular needle 34 and distal end 30 of the torquable shaft 26. The first helical tissue penetration member 324 has a sharpened distal tip 332 configured to penetrate tissue upon rotation of the tissue penetration member 322. The second helical tissue penetration member 328 has a sharpened distal tip 334 configured to penetrate tissue upon rotation of the tissue penetration member 322. The first and second helical tissue penetration members 324 and 328 provide opposing forces which cancel each other to a certain extent and minimize the lateral deflection of the tissue penetration member 322 during rotation and tissue penetration member 322. Sharpened distal tips 332 and 334 of the helical tissue penetration members 324 and 328 are disposed opposite the tubular needle 34 180 degrees apart and oriented such that the sharpened tips 332 and 334 are disposed about 90 degrees from the distal extremity of the sharpened tip 38 of the tubular needle 34.

FIGS. 30-36 illustrate the activation modulator 312 for applying controlled axial movement and rotation to the tissue penetration member 32 and limiting the rotational movement of the tissue penetration member 32. The activation modulator 312 has a fixed member in the form of an outer barrel 334 which has a threaded portion 336 shown if FIG. 34. A rotating member in the form of an inner barrel 338 has a threaded portion 340 that is engaged with the threaded portion 336 of the outer barrel 334. The inner barrel 338 has a distal surface 342 and annular flange 344 which are axially captured within a cavity 346 of the outer barrel 334 shown in FIG. 34. FIG. 34 shows the threaded inner barrel 340 disposed at a proximal limit of axial movement wherein a proximal surface of the annular flange 344 is engaged with a distal surface of an annular flange 348 of the outer barrel 334. FIG. 36 shows the threaded inner barrel 338 disposed at a distal limit of axial movement with the distal surface 342 engaged with a distal cavity surface 350 of the outer barrel 334. The distance from distal surface 342 to distal cavity surface 350 controls or limits the depth of penetration of the tissue penetration member 32.

FIG. 35 is an enlarged view of the rotation seal 352 of the inner barrel 338 disposed within an annular groove 354 of the threaded inner barrel. The rotation seal 352 may be an annular seal such as an o-ring type seal that is secured within the annular groove 354 and is sized to seal against an inner surface 356 of the proximal portion of the cavity 346 of the outer barrel 334. The rotation seal 352 provides a fluid seal between the outer surface of the inner barrel 338 and the cavity 346 while allowing relative rotational movement between the inner barrel 338 and outer barrel 334.

The outer barrel 334 has a substantially tubular configuration with a Luer type fitting 358 at the distal end 360 of the outer barrel 334. The Luer fitting 358 can be used to secure the activation modulator 312 in a fluid tight arrangement to a standard guide catheter 14 having a mating Luer connector arrangement on a distal end thereof. The outer barrel 334 also has a side port 360 which is in fluid communication with an inner lumen 362 disposed within the distal end of the outer barrel 334. The side port 360 can be used to access the space between the outer surface of the torquable shaft 26 and inner surface of the guide catheter lumen for injection of contrast media and the like. The outer barrel 334 has a series of longitudinal slots 364 that allow the annular flange 348 portion of the outer barrel 334 to expand radially for assembly of the inner barrel 338 into the cavity 346 of the outer barrel 334.

The inner barrel 338 has a knurled ring 366 that may be useful for gripping by a user in order to manually apply torque to the inner barrel 338 relative to the outer barrel 334. A threaded compression cap 368 having a threaded portion 370 is configured to engage a threaded portion 372 of the inner barrel 338, as shown in FIG. 34. The compression cap 368 has an inner lumen to accept the torquable shaft 26 of the tissue penetration device. A sealing gland 374 having a substantially tubular configuration and an inner lumen configured to accept the torquable shaft 26 is disposed within a proximal cavity 376 of the inner barrel 338 and can be compressed by the compression cap 368 within the proximal cavity 376 such that the sealing gland 374 forms a seal between an inner surface of the proximal cavity 376 and an outer surface of the torquable shaft 26. The compressed sealing gland 374 also provides mechanical coupling between the inner barrel 338 and the torquable shaft 26 so as to prevent relative axial movement between the torquable shaft 26 and the inner barrel 338. The sealing gland may be made from any suitable elastomeric material that is sufficiently deformable to provide a seal between the proximal cavity 376 and the torquable shaft 26. A distal inner lumen 380 of the inner barrel 338 is keyed with a hexagonal shape for the transverse cross section of the inner lumen 380 which mates with a hexagonal member 382 secured to the outer surface of a proximal portion of the torquable shaft 26 so as to allow relative axial movement between the hexagonal member 382 and the inner lumen 380 but preventing relative rotational movement. This arrangement prevents rotational and axial slippage between the inner barrel 338 and the torquable shaft 26 during rotational activation of the activation modulator 312.

Axial movement or force on the tissue penetration member is generated by the activation modulator 312 upon relative rotation of the inner barrel 338 relative to the outer barrel 334. The axial movement and force is then transferred to the tissue penetration member 32 by the torquable shaft 26. The pitch of the threaded portions may be matched to the pitch of the helical tissue penetration member 42 so that the tissue penetration member 32 is forced distally at a rate or velocity consistent with the rotational velocity and pitch of the helical tissue penetration member 42.

For use of the transmembrane access system 310, the distal end of the guide catheter 14 is positioned adjacent a desired target tissue site in a manner similar to or the same as discussed above with regard to the transmembrane access system 10. The tissue penetration member 32 of the tissue penetration device is then advanced until the distal tip 38 of the tissue penetration member 32 is disposed adjacent target tissue. The torquable shaft 26 is then secured to the inner barrel 338 of the activation modulator 312 by the sealing gland 374 with the inner barrel disposed at a proximal position within the cavity 346 of the outer barrel 334. The user then grasps the knurled ring 366 and rotates the ring 366 relative to the outer barrel 334 which both rotates and advances both the inner barrel 338 relative to the outer barrel 334. This activation also rotates and distally advances the torquable shaft 26 and tissue penetration member 32 relative to the guide catheter 14. The rotational activation of the activation modulator can be continued until the distal surface 342 of the inner barrel 338 comes into contact with the surface 350 of the outer barrel 334. The axial length of the cavity 346 can be selected to provide the desired number of maximum rotations and axial advancement of the torquable shaft 26 and tissue penetration member 32. In one embodiment, the maximum number of rotations of the inner barrel 338 relative to the outer barrel 334 can be from about 4 rotations to about 10 rotations.

The tissue penetration device 16 discussed above may have a variety of configurations and constructions. FIGS. 37-39 illustrate another embodiment of a tissue penetration device 410. The tissue penetration device 410 has a construction and configuration that is similar in some ways to the tissue penetration device 16 discussed above. The tissue penetration device 410 has a tissue penetration member 412 secured to a distal end of a torquable shaft 414. A keyed hexagonal member 382 is secured to a proximal portion of the torquable shaft 414 for coupling with the activation modulator 312 discussed above. The distal portion 416 of the tissue penetration device 410 has a flexible construction that includes a helical coil member 418 disposed within a braided tubular member 420, both of which are covered by a polymer sheath 422 that provides a fluid tight lumen to contain fluids passing therethrough. The proximal portion of the torquable shaft 414 is made from a tubular member 424 of high strength material, such as a hypodermic tubing of stainless steel. The distal end 426 of the tubular member is secured to the proximal ends of the helical coil member 416 and braided tubular member 420 by any suitable method such as soldering, brazing, welding, adhesive bonding or the like.

A tubular needle 34 forms the center of the tissue penetration member 412 along with the distal portion 428 of the helical coil member 418 which is configured as a helical tissue penetration member disposed about the tubular needle 34. The proximal end 430 of the tubular needle 34 is secured to the helical coil member 418 and braided tubular member 420 by any suitable method such as soldering, brazing, welding, adhesive bonding or the like. The polymer sheath 422 may be bonded to the outer surface of the braided tubular member 420 or mechanically secured to the braided tubular member by methods such as heat shrinking the polymer sheath material over the braided tubular member 420. The flexible distal section 416 can have any suitable length. In one embodiment, the flexible distal section has a length of about 15 cm to about 40 cm. The configuration, dimensions and materials of the tissue penetration member 412 can be the same as or similar to the configuration, dimensions and materials of the tissue penetration members 32 and 322 discussed above.

FIGS. 40-42 illustrate another embodiment of a tissue penetration device 430 having a construction similar to that of the tissue penetration device 410 except that the tubular member 432 of the torquable shaft 434 extends continuously from the proximal end 436 of the device 430 to the distal end 438 and the helical coil member 418 of the tissue penetration device 410 has been replaced with a flexible section 438 of the tubular member 432. The flexible section 438 is made by producing a series of adjacent alternating partial transverse cuts into the tubular member 432 so as to allow improved longitudinal flexibility of the tubular member 432 in the flexible section 438 while maintaining the radial strength of the tubular member 432. The flexible section 438 is covered by a braided tubular member 440 and a polymer sheath 442. The braided tubular member 440 may be secured at its proximal end and distal end 444 by soldering, brazing, welding, adhesive bonding or the like. The polymer sheath 442 may be secured by adhesive bonding, thermal shrinking or the like. The tissue penetration member 446 includes a helical tissue penetration member 448 secured at its proximal end to the tubular member 432 which terminates distally with a sharpened tip 448 in a configuration similar to the tissue penetration members discussed above. The configuration, dimensions and materials of the tissue penetration member 446 can be the same as or similar to the configuration, dimensions and materials of the tissue penetration members 32 and 322 discussed above. In addition, tissue penetration devices 410 and 430 both have inner lumen extending the length thereof for passage of the guidewire 18 or other elongate member having properties similar to or the same as a guidewire 18.

FIG. 43 shows an embodiment of a transmembrane access system 10A that is similar to the transmembrane access system 10 discussed above and includes some of the same components. Transmembrane access system 10A includes a stabilizer sheath 12A that has a “pigtail” curled distal tip 501 laterally curling away from and extending opposite the side port 22. This configuration allows the curled distal tip 501 to brace against supporting tissue of a patient and further stabilize the side port 22 of the stabilizer sheath 12A in the radial orientation of the side port 22. The stabilizer sheath 12A has an inner lumen 504 extending through the length of the stabilizer sheath 12A and side port 22 disposed on a distal section thereof which is in fluid communication with the inner lumen 504. The curled “pigtail” section or tip 501 terminates distally at a distal end of the elongate tubular shaft with port 70A. In some embodiments, port 70A may have a discharge axis that is greater than 180 degrees from a longitudinal axis of the elongate tubular shaft 12A proximal of the curled section 501. As noted above, the curled section 501 is directed substantially opposite the side port 22 with respect to circumferential orientation about the stabilizer sheath 12A.

The tubular guide catheter 14 has a shaped distal section 24 with a curved configuration in a relaxed state and an outer surface which is configured to move axially within a portion of the inner lumen 504 of the stabilizer sheath 12A that extends from the proximal end of the stabilizer sheath 20A to the side port 22. The tissue penetration device 16 is configured to move axially within an inner lumen 506 of the tubular guide catheter 14 and is axially extendable from the guide catheter 14 for membrane penetration. Although rotationally actuated tissue penetration device 16 is illustrated with the access system 10A, other tissue penetration devices, such as those discussed above with regard to copending application Ser. No. 10/889,319, could also be used.

Ultrasound imaging may also be used with the access system 10A in order to facilitate positioning of the guide catheter 14 during a procedure. FIGS. 43-45 show first ultrasound transducer 17A and second ultrasound transducer 17B in communication with or electrically coupled to ultrasound signal controller 15A and display member 15B. This allows ultrasound energy or signals to be emitted from the ultrasound transducers 17A and 17B into the space and tissue surrounding the access device 10A during a procedure in a substantially radial direction, or any other desired direction, towards the side port 22, as shown by arrows 508 in FIG. 45. The transducers 17A and 17B may be secured to the stabilizer sheath 12A, or any other suitable portion of the access system 10A in order to project an ultrasound signal in a desired direction. The configuration shown allows imaging of space and tissue in the direction of the side port 22 which may be used to confirm the location of a portion of the access system 10A, such as the distal tip of the guide catheter 14, relative to a desired site or structure of surrounding tissue, such as the atrial septum. If a scanning phased array type transducer is used, a two dimensional image of tissue adjacent the side port 22 or guide catheter 14 may be obtained. Features or information such as tissue type, depth, tissue surface distance from the side port, guide catheter 14 orientation, tissue penetration device orientation and the like may be obtained from the reflected ultrasound signal or energy. As with the systems discussed above, guidewire 18 may include an optional sensor 18A disposed at a distal end of the guidewire 18. The sensor 18A may be an ultrasound transducer, electrode or the like. The sensor 18A may be used to gather information about the space or tissue adjacent the distal end of the guidewire 18 as discussed above. Transmembrane access system 10A may be used in a manner which is similar to or the same as the methods and procedures discussed above with regard to transmembrane access system 10.

FIG. 46 shows another embodiment of a transmembrane access system 10B having a stabilizer sheath 12B with a stabilizer member or guidewire 203 extending from the distal end 510 of the stabilizer sheath 12B for lateral support of the stabilizer sheath. The guide catheter 14 extends distally from a distal port 70B of the stabilizer sheath 12B. The stabilizer sheath 12B has an inner work lumen 512 extending the length thereof from the distal end 510 of the stabilizer sheath 12B to a proximal end 20B of the stabilizer sheath 12B. The port 70B is disposed at the distal end 510 on a distal section 514 of the stabilizer sheath and is in fluid communication with the inner lumen 512. A stabilizer member lumen 516, shown in FIG. 47, is disposed substantially parallel to a nominal longitudinal axis of the stabilizer sheath 12B. The stabilizer member lumen 516 extends proximally from a distal port 517 of the stabilizer member lumen 516 to a Y-adapter 515 at a proximal end 20B of the stabilizer sheath 12B. In the embodiment shown, the distal port 70A of the stabilizer sheath 12B and distal port 517 of the stabilizer member lumen 516 are substantially coextensive with respect to the longitudinal axis of the stabilizer sheath 12B. An elongate stabilizer member in the form of a guidewire 203 is configured to extend from the distal port 517 of the stabilizer member lumen 516 and provide lateral support to the distal end 510 of the stabilizer sheath 12B. Guidewire 203 is shown extending from the Y-adapter 515 to the distal end 510 of the stabilizer sheath 12B.

Guide catheter 14 has a shaped distal section 24 with a curved configuration in a relaxed state and an outer surface which is configured to move axially within a portion of the inner work lumen 512 of the stabilizer sheath 12B that extends from the proximal end of the stabilizer sheath 12B to the port 70B. Tissue penetration device 16 is configured to move axially within the inner lumen of the tubular guide catheter 14 and is axially extendable from the guide catheter 14 for membrane penetration. A first ultrasound transducer 17A is disposed on a distal portion 514 of the stabilizer sheath 12B and is electrically coupled to the ultrasound signal controller 15A which is electrically coupled to the display member in the form of a video monitor 15B. Guidewire 18 may also include an optional sensor 18A as discussed above with regard to other embodiments.

FIG. 48 illustrates the transmembrane access system 10B of FIG. 46 with a distal end 510 of the stabilizer sheath 12B disposed within a vena cava 518 of a patient. Guide catheter 14 and tissue penetration device 16 of the system 10B extend from the vena cava 518 and into the right atrium of the patient (not shown). The distal end 510 of the stabilizer sheath 12B is shown disposed in the superior vena cava 520 and the elongate stabilizer member in the form of guidewire 203 extends from the distal end 510 of the stabilizer sheath and down into the inferior vena cava 522 in order to provide lateral support to and stabilize the position of the port 70B of the stabilizer sheath 12B. In one embodiment of use, the stabilizer sheath 12B is advanced through the superior vena cava 520 of the patient and positioned with the elongate stabilizer member 203 within the inferior vena cava 522. The port 70B of the stabilizer sheath 12B is positioned adjacent the right atrium (not shown) or other desired location within the patient's body. The distal end 66 of the guide catheter 14 is then advanced through the inner work lumen 512 of the stabilizer sheath 12B until the distal end 66 of the guide catheter 14 is positioned adjacent a desired site of the septum of the patient's heart (not shown). Positioning of the port 70B or guide catheter 14 may be facilitated by use of the ultrasound imaging system wherein ultrasound energy is emitted from the first ultrasound transducer 17A in the direction of a desired location adjacent the access system 10B. A reflected ultrasound signal or energy is then received by the transducer 17A and converted into an image or other useful information by the ultrasound signal controller or processor 15A which is displayed on the display member 15B. The tissue penetration member 32 of the tissue penetration device 16 is then advanced from the distal end 66 of the guide catheter 14. The tissue penetration member 32 is then actuated and advanced distally through the septum. Activation may include rotational movement with optional axial advancement for a tissue penetration sequence similar to or the same as those discussed above.

FIG. 49 shows another embodiment of a stabilizer sheath 12C of the transmembrane access system 10B of FIG. 46. In this embodiment, a stabilizer member or guidewire 203 used to stabilize the distal portion 526 of the stabilizer sheath 12C is slidingly disposed within a short lumen 524 at the distal portion 526 of the stabilizer sheath 12C. This configuration allows the stabilizer sheath 12C to be advanced over a standard length guidewire 203 that is already positioned within a patient's body. The length of the short lumen 524 may be less than about one half the overall length of the stabilizer sheath 12C, but may also be less than about 10 cm.

FIGS. 50-53 illustrate an embodiment of a transmembrane access system 10C that includes a stabilized guide catheter 14A having a stabilizer member lumen 530 that extends proximally from a distal portion 532 of the guide catheter 14A. An elongate stabilizer member that may be in the form of guidewire 203 for stabilizing the distal portion 532 of the guide catheter 14A is disposed within the stabilizer member lumen 530 and is free to slide in an axial direction within the stabilizer member lumen 530. The stabilized guide catheter 14A has a shaped distal section 24A that includes a curved configuration in a relaxed state that may also have a configuration of curvature that is the same as, or similar to, the curvature of the guide catheter embodiments discussed above. The guide catheter 14A has an inner work lumen 533 extending therein. A distal port 534 of the inner work lumen 533 is disposed at a distal end 540 of the stabilized guide catheter 14A and is in fluid communication with the inner work lumen 533. The inner work lumen 533 is configured to allow passage of a tissue penetration device such as tissue penetration device 16.

The stabilizer member lumen 530 is substantially parallel to a nominal longitudinal axis of the stabilized guide catheter 14A proximal of the shaped distal section 24A. The stabilizer member lumen 530 has a distal port 530A that is disposed immediately proximal of the shaped distal section 24A of the guide catheter 14A with the stabilizer member lumen extending proximally to a Y-adapter 536. The elongate stabilizer member 203 extends distally from the distal port 530A of the stabilizer member lumen 530 of the guide catheter 14A and provides lateral support to the distal portion 532 of the guide catheter 14A, and particularly of the shaped distal section 24A of the distal portion 532. The position of the distal port 530A just proximal to the shaped distal section 24A allows the shaped distal section 24A to assume its curved configuration while being stabilized by the stabilization member 203. Tissue penetration device 16 is configured to move axially within the inner work lumen 533 and is axially extendable from a distal port 534 of the inner work lumen 533 of the stabilized guide catheter 14A for membrane penetration. The materials, dimensions and features of the stabilized guide catheter 14A may be the same as or similar to those of guide catheter 14 discussed above. Transmembrane access can be carried out with the stabilized guide catheter 14A and tissue penetration device 16 disposed within the stabilized guide catheter 14A without the use of a separate stabilizer sheath 12.

FIG. 53 shows the transmembrane access system 10C disposed within a vena cava 518 of a patient with the distal end 540 of the stabilized guide catheter 14A extending from the vena cava 518 and into the right atrium (not shown). A distal portion of the tissue penetration device 16 is shown extending from the distal port 534 of the guide catheter 14A. In one embodiment of use, the stabilized guide catheter 14A is advanced through the superior vena cava 520 of the patient over a guidewire (not shown) disposed within the inner work lumen 533 of the guide catheter 14A. This guidewire is then removed and the distal end 540 of the guide catheter 14A is then positioned adjacent a desired site of the septum of the patient's heart (not shown). Positioning of the distal port 534 or of the stabilized guide catheter 14A may be facilitated by use of the ultrasound imaging system wherein ultrasound energy is emitted from the first ultrasound transducer 17A in the direction of a desired location adjacent the system 10C as indicated by arrows 542 in FIG. 53. A reflected ultrasound signal or energy is then received by the transducer 17A and converted into an image or other useful information by the ultrasound signal controller or processor 15A which is displayed on the display member 15B. The elongate stabilizer member in the form of guidewire 203 is then advanced distally through the stabilizer member lumen 530 until the elongate stabilizer member 203 extends out of the distal port 530A of the stabilizer member lumen and into the inferior vena cava 522 to provide support to the distal portion 532, shaped distal section 24A and the distal tip 540 of the stabilized guide catheter 14A. The tissue penetration member 32 of the tissue penetration device 16 is then advanced from the distal of the stabilized guide catheter 14A. The tissue penetration member 32 is then actuated and advanced distally through the septum.

FIG. 54 shows an enlarged view of a distal portion 539 of another embodiment of a stabilized guide catheter 14B. The stabilized guide catheter 14B has an optional shortened stabilizer member lumen 538. The stabilizer member in the form of guidewire 203 is slidingly disposed within the short stabilizer member lumen 538 at a distal portion 539 of the guide catheter 14B. The stabilizer member lumen 538 has a distal port 538A and extends proximally from the distal port 538A to a proximal port 538B. For some embodiments, the distal port 538A may be disposed just proximal of the shaped distal section 24A of the stabilized guide catheter 14B. The length of the short stabilizer member lumen 538 can be from about 5 cm to about 20 cm. In some embodiments, the length of the short lumen 538 may be less than about one half the overall length of the guide catheter 14B, but may also be less than about 10 cm. This configuration allows the guide catheter 14B to be advanced over a standard length guidewire that is already positioned within a patient's body without disturbing the position of the guidewire. This is carried out by inserting the proximal end of the stabilizer member 203 into the distal port 538A of the stabilizer member lumen 538 outside the patient's body. The stabilized guide catheter can then be advanced distally over the stabilizer member 203 into the patient's body while holding the proximal portion of the stabilizer member in a fixed longitudinal position. Other than the shortened stabilizer member lumen, the features and methods of use of the stabilized guide catheter 14B may be the same as or similar to those of stabilized guide catheter 14A.

FIGS. 55-56 illustrate a distal portion of a stabilized guide catheter system 550 that includes a stabilized guide catheter 14C having an inner work lumen 552 and a distal port 554 disposed in fluid communication with the inner work lumen 552. The inner work lumen may be configured to accept a tissue penetration device, such as tissue penetration device 16. The stabilized guide catheter includes a shaped distal section 24C that has a curved configuration in a relaxed state. A stabilizer member lumen 556 is disposed substantially parallel to a longitudinal axis 558 of the guide catheter 14C and extends proximally from an intermediate port 560 of the stabilizer member lumen 556 to a proximal port 557 of the stabilizer member lumen. The intermediate port 560 is disposed just proximal to the shaped distal section 24C of the guide catheter 14C. The stabilizer member lumen 556 also extends distally from the intermediate port 560 to a distal port 562 of the stabilizer member lumen which is disposed in the shaped distal section 24C of the guide catheter 14C. In the embodiment shown, the distal port 562 of the stabilizer member lumen 556 is axially coextensive with a distal port 554 of the inner work lumen 552 and the distal end 564 of the stabilized guide catheter 14C. The materials, dimensions and features of the stabilized guide catheter 14C may be the same as or similar to those of guide catheter 14B discussed above.

An elongate stabilizer member 203 in the form of a guidewire is configured to extend distally from the intermediate port 560 to provide lateral support to a distal portion 564 of the guide catheter. The stabilizer member 203 is also configured to extend distally from the distal port 562 of the stabilizer member lumen 556 where the stabilizer member 203 may serve to straighten the shaped distal section 24C of the guide catheter during delivery of the system to a desired site in a patient's body. The stabilizer member 203 may have a longitudinal stiffness in a distal portion thereof that is selected to have sufficient flexibility to allow delivery of the member 203 and guide catheter 14C into a desired site within a patient's body, but still retain sufficient stiffness to force the shaped distal section 24C to conform, at least partially, to the straight configuration of the stabilizer member 203. During delivery of the system, the stabilizer member 203 may also serve a guiding function as a guidewire when exiting the distal port 562.

The intermediate port 560 in the embodiment shown is disposed just proximal to a proximal boundary of the shaped distal section 24C of the guide catheter 14C, however, the intermediate port 560 could be disposed slightly distal of the proximal boundary of the shaped distal section 24C or proximal of the proximal boundary of the shaped distal section by an amount that will still provide lateral support to the distal portion of the guide catheter 14C when the stabilizer member 203 is deployed. In the embodiment shown, the stabilizer member lumen 556 is a short lumen extending proximally from the distal port 562 of the stabilizer member lumen to the proximal port 557 over a length less than about one half the overall length of the guide catheter 14C. In other embodiments, the stabilizer member lumen extends proximally from the distal port 562 a length less than about 10 cm.

In use, the stabilized guide catheter 14C is advanced into a patient with the stabilized guide catheter 14C tracking over the stabilizer member 203 which is disposed within the stabilizer member lumen 556 from the proximal port 557 to the distal port 562. During this advancement, the shaped distal section 24C of the guide catheter is held in a substantially straightened configuration by the longitudinal stiffness of the stabilizer member 203 disposed within the stabilizer member lumen portion from the intermediate port 560 to the distal port 562. When the distal end of the guide catheter is disposed appropriately for allowing the curvature of the shaped distal section 24C to deploy, the stabilizer member 203 is withdrawal proximally until the distal end of the stabilizer member 203 is proximal of the intermediate port 560. At this point, the shaped distal section 24C can assume or approximately assume the curvature of the shaped distal section 24C in a relaxed state and deflect laterally a predetermined angular displacement. The stabilizer member 203 can then be advanced distally in the stabilizer member lumen 556 until the distal end of the stabilizer member 203 exits the intermediate port 560. The stabilizer member can then be further advanced distally from the intermediate port 560, as shown in FIG. 56, until positioned to provide lateral stabilization to the shaped distal section 24C and distal portion 564 of the guide catheter 14C. Once the distal portion 564 of the guide catheter 14C is stabilized, a tissue penetration device 16 may be advanced through the inner work lumen 556 and extended beyond the distal port 554 of the inner work lumen to tissue to perform tissue or membrane penetration.

FIGS. 57-63 illustrate a stabilized guide catheter system 580 and method of using the system. The stabilized guide catheter system 580 includes a stabilized guide catheter 14D which may have materials, dimensions and features which are the same as or similar to those of stabilized guide catheter 14B. The stabilized guide catheter 14D includes an inner work lumen 582 and a distal port 584 disposed at a distal end of the stabilized guide catheter 14D and in fluid communication with the inner work lumen 582. The stabilized guide catheter 14D has a shaped distal section 24D that includes a curved configuration in a relaxed state. A stabilizer member lumen 586 is disposed substantially parallel to a nominal longitudinal axis 588 of the stabilized guide catheter 14D and extends proximally from a distal port 590 of the stabilizer member lumen 586 to a proximal port 594 of the stabilizer member lumen 586. The distal port 590 is disposed just proximal to the shaped distal section 24D of the guide catheter 14D. The distal port 590 in the embodiment shown is disposed just proximal to the proximal boundary of the shaped distal section 24C of the guide catheter 14C, however, the distal port 590 could be disposed slightly distal of the proximal boundary of the shaped distal section 24C or proximal of the proximal boundary of the shaped distal section by an amount that will still provide lateral support to the distal portion 592 of the guide catheter 14C when the stabilizer member 203 is deployed. Also, in the embodiment shown, the stabilizer member lumen 586 is a short lumen extending proximally from the distal port 590 of the stabilizer member lumen 586 to a proximal port 594 over a length less than about one half the overall length of the guide catheter 14D. In other embodiments, the stabilizer member lumen extends proximally from the distal port 590 a length less than about 10 cm.

The elongate stabilizer member 203 is configured to extend from the distal port 590 of the stabilizer member lumen 586 and provide lateral support to a distal portion 592 of the stabilized guide catheter 14D. In addition, the stabilized guide catheter system 580 may also include an elongate dilator 596 configured to slide axially within the working lumen 582 of the guide catheter 14D. The elongate dilator has a distal portion 597 that includes a distal stabilizer member lumen 598, as shown in FIG. 60, which is configured to allow axial passage of the elongate stabilizer member 203. The distal stabilizer member lumen 598 includes a proximal port 600 and distal port 602 both of which are configured and positioned on the dilator 596 to extend beyond a distal end 604 of the stabilized guide catheter 14D such that the proximal port 600 and distal port 602 of the distal stabilizer member lumen 598 are accessible for loading of a stabilizer member 203 therethrough. In the embodiment shown, the proximal port 600 of the distal stabilizer member lumen 598 of the dilator 596 opens to the side of the dilator 596 and the distal port 602 of the distal stabilizer member lumen 598 opens in a distal direction from a distal tip 604 of the elongate dilator 596. Also in the embodiment shown, the stabilizer member lumen 586 is a short lumen extending proximally from the distal port 590 of the stabilizer member lumen 586 to the proximal port 594 for a length less than about one half the overall length of the guide catheter 14D. In other embodiments, the length of the stabilizer member lumen 586 is less than about 10 cm.

In use, the stabilizer member 203 is first loaded into the stabilizer member lumen 586 and distal stabilizer member lumen 598 of the elongate dilator 596 with the stabilizer member 203 extending distally from the distal port 602 of the distal stabilizer member lumen 598 as shown in FIG. 60. The distal end 604 of the guide catheter 14D and distal portion 597 of the elongate dilator 596 can be advanced into a patient's vasculature 606, as shown in FIG. 61, and steered over the stabilizer member 203 which performs a guidewire function during the initial portion of the procedure. Once the distal tip 604 of the guide catheter has been positioned in a desired area 608 of the patient's vasculature, the stabilizer member 203 can be withdrawn proximally until it is removed from the distal stabilizer member lumen 598 of the dilator. The stabilizer member 203 can be further retracted proximally until the distal end of the stabilizer member 203 is directed into a portion of the patient's vasculature 606 substantially in line with the stabilizer member lumen 586. The stabilizer member may then be advanced again so as to perform a stabilization function for the distal portion 592 of the guide catheter 14D, as shown in FIG. 62. Once this positioning has been achieved, the dilator 596 may be retracted proximally, as shown in FIG. 63, and a tissue penetration device 16 or other device advanced through the inner work lumen 584 of the guide catheter 14D and used for tissue or membrane penetration and access to the other side of the tissue or membrane (not shown). Use of the ultrasound imaging system which includes ultrasound transducer 17A as well as the components 15A and 15B that are used to generate, process and display the ultrasound signal, may be incorporated into the procedure prior to final positioning of the distal end 604 of the guide catheter 14D, advancement of the tissue penetration device, or at any other suitable time during the procedure.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A transmembrane access system, comprising:
a stabilizer sheath having an inner lumen extending therein and having a side port disposed on a distal section of the stabilizer sheath and in communication with the inner lumen;
a guide catheter having a shaped distal section that has a curved configuration in a relaxed state and an outer surface which is configured to move axially within a portion of the inner lumen of the stabilizer sheath that extends from the proximal end of the stabilizer sheath to the side port;
a tissue penetration member which is configured to move axially within an inner lumen of the guide catheter and which is axially extendable from the guide catheter for membrane penetration; and
an ultrasound emission member and an ultrasound receiver disposed at the distal section of the stabilizer sheath.

2. The system of claim 1 wherein the ultrasound emission member and ultrasound receiver comprise an ultrasound transducer disposed on the distal section of the stabilizer sheath.

3. The system of claim 2 further comprising an ultrasound signal controller in communication with the ultrasound transducer and a display member in communication with the ultrasound signal controller.

4. The system of claim 1 wherein the tissue penetration member is configured to penetrate tissue upon rotation and the system further comprises an elongate torquable shaft coupled to the tissue penetration member.

5. A stabilizer sheath, comprising
an elongate shaft having an inner lumen;
a side port disposed in a distal section of the elongate tubular shaft in fluid communication with the inner lumen;
a deflecting surface disposed in the inner lumen opposite the side port; and
an ultrasound emission member and an ultrasound receiver disposed at the distal section of the stabilizer sheath system.

6. The stabilizer sheath of claim 5 wherein the ultrasound energy generator and ultrasound energy receiver comprise an ultrasound transducer disposed on the distal section of the stabilizer sheath.

7. The stabilizer sheath of claim 5 wherein a distal portion of the distal section comprises a curled section wherein the discharge axis of the distal end of the elongate tubular shaft is greater than 180 degrees from the longitudinal axis of the elongate tubular shaft proximal of the curled section.

8. The stabilizer sheath of claim 7 wherein the curled section is disposed opposite the side port with respect to a circumferential orientation about the stabilizer sheath.

9. A transmembrane access system, comprising:
a guide catheter having a shaped distal section that has a curved configuration in a relaxed state;
a tissue penetration member which is axially extendable from a distal end of the guide catheter for membrane penetration; and
an ultrasound emission member and an ultrasound receiver disposed adjacent the shaped distal section of the guide catheter.

10. The system of claim 9 wherein the ultrasound emission member and ultrasound receiver comprise an ultrasound transducer.

11. The system of claim 9 wherein the tissue penetration member comprises a tubular needle with a sharpened distal end and helical tissue penetration member disposed about the hypodermic needle.

12. A method of accessing the left atrium of a patient's heart from the right atrium of the patient's heart, comprising
providing a transmembrane access system, including:
a stabilizer sheath having an inner lumen extending therein and having a side port disposed on a distal section of the sheath and in communication with the inner lumen,
a guide catheter having a shaped distal section that has a curved configuration in a relaxed state and an outer surface which is configured to move axially within a portion of the inner lumen of the stabilizer sheath that extends from the proximal end of the stabilizer sheath to the side port,
a tissue penetration member which is configured to move axially within an inner lumen of the guide catheter and which is axially extendable from a distal end of the guiding catheter for membrane penetration, and
an ultrasound emission member and an ultrasound receiver disposed at the distal section of the stabilizer sheath;
advancing the stabilizer sheath through a superior vena cava of the patient and positioning the stabilizer sheath with the distal end of the sheath within the inferior vena cava with the side port of the stabilizer sheath facing the right atrium of the patient's heart;
advancing the distal end of the guide catheter through the inner lumen of the stabilizer sheath and out of the side port until the distal end of the guide catheter is positioned adjacent a desired site of the septum of the patient's heart;
advancing the tissue penetration member from the distal end of the guide catheter;
emitting ultrasound energy from the ultrasound emission member directed towards the desired site;
receiving reflected ultrasound energy with the ultrasound receiver;
generating information about the desired site from the reflected ultrasound energy; and
activating the tissue penetration member and advancing the tissue penetration member distally through the septum.

13. A method of accessing a second side of a tissue membrane from a first side of a tissue membrane, comprising
providing a transmembrane access system, having
a guide catheter with a shaped distal section that has a curved configuration in a relaxed state,
a tissue penetration member which is disposed within a distal end of the guide catheter and which is axially extendable from the distal end of the guide catheter for membrane penetration, and
an ultrasound emission member and ultrasound receiver disposed at a distal portion of the guide catheter;
positioning the distal end of the guide catheter until the distal end of the guide catheter is adjacent a desired site on the first side of the tissue membrane;
advancing the tissue penetration member distally from the guide catheter until the distal end of the tissue penetration member is adjacent the tissue membrane;
emitting ultrasound energy from the ultrasound emission member directed to the desired site;
receiving reflected ultrasound energy from the desired site with the ultrasound receiver and generating information about the tissue membrane from the reflected ultrasound energy; and
activating the tissue penetration member so as to penetrate distally through the tissue membrane.

14. The method of claim 13 further comprising advancing a guidewire through the guidewire lumen of the tissue penetration member until a distal end of the guidewire is disposed on the second side of the tissue membrane.

15. The method of claim 13 wherein the ultrasound emission member and ultrasound receiver comprise an ultrasound transducer directed substantially toward the distal end of the guide catheter and wherein the system further comprises an ultrasound signal controller in communication with the ultrasound transducer and a display member in communication with the ultrasound signal controller and wherein the ultrasound energy directed toward the desired site is generated by the transducer and the reflected ultrasound energy is received by the transducer and the information generated about the tissue membrane is displayed on the display member.

16. A method of positioning an access catheter within a chamber of a patient's body, comprising
providing an access system, including:
a stabilizer sheath having a tubular configuration with an inner lumen extending therein and having a side port disposed on a distal section of the stabilizer sheath and in communication with the inner lumen,
a guide catheter having a shaped distal section that has a curved configuration in a relaxed state and an outer surface which is configured to move axially within a portion of the inner lumen of the stabilizer sheath that extends from the proximal end of the stabilizer sheath to the side port, and
an ultrasound emission member and an ultrasound receiver disposed at the distal section of the stabilizer sheath;
advancing the stabilizer sheath through a first tubular structure of the patient which is in fluid communication with the chamber and positioning the stabilizer sheath with the side port of the stabilizer sheath adjacent the chamber of the patient's body and with a portion of the stabilizer sheath distal of the side port into a second tubular structure which is also in fluid communication with the chamber;
advancing the distal end of the guide catheter through the inner lumen of the stabilizer sheath until the distal end of the guide catheter exits the side port of the stabilizer sheath;
emitting ultrasound energy from the ultrasound emission member directed towards a desired site within the chamber;
receiving reflected ultrasound energy with the ultrasound receiver;
generating information about the desired site from the reflected ultrasound energy;
positioning the distal end of the guide catheter is positioned adjacent the desired site of the chamber.

17. A transmembrane access system, comprising:
a stabilizer sheath having an inner lumen extending therein, having a side port disposed on a distal section of the sheath and in communication with the inner lumen and having a curled section on a distal portion of the distal section wherein the discharge axis of the distal end of the elongate tubular shaft is greater than 180 degrees from the longitudinal axis of the stabilizer sheath proximal of the curled section and wherein the curled section is directed opposite the side port with respect to circumferential orientation about the stabilizer sheath;
a guide catheter having a shaped distal section that has a curved configuration in a relaxed state and an outer surface which is configured to move axially within a portion of the inner lumen of the stabilizer sheath that extends from the proximal end of the stabilizer sheath to the side port; and
a tissue penetration member which is configured to move axially within an inner lumen of the tubular guide catheter and which is axially extendable from a distal end of the guide catheter for membrane penetration.

18. The system of claim 17 wherein the tissue penetration member is configured to penetrate tissue upon rotation.

19. The system of claim 17 further comprising an ultrasound emission member and an ultrasound receiver disposed at the distal section of the stabilizer sheath.

20. The system of claim 19 wherein the ultrasound energy generator and ultrasound energy receiver comprise an ultrasound transducer.

21. A transmembrane access system, comprising:
a stabilizer sheath having an inner work lumen extending therein, having a port disposed on a distal end of the sheath and in communication with the inner lumen and having a stabilizer member lumen substantially parallel to a longitudinal axis of the stabilizer sheath disposed at the distal section;
an elongate stabilizer member that is configured to extend distally from the stabilizer member lumen and provide lateral support to the distal end of the stabilizer sheath;
a guide catheter having a shaped distal section that has a curved configuration in a relaxed state and an outer surface which is configured to move axially within a portion of the inner work lumen of the stabilizer sheath that extends from the proximal end of the stabilizer sheath to the port; and
a tissue penetration member which is configured to move axially within an inner lumen of the tubular guide catheter and which is axially extendable from a distal end of the guide catheter for membrane penetration.

22. The system of claim 21 wherein the stabilizer member lumen comprises a guidewire lumen and the stabilizer member comprises a guidewire configured to translate axially within the guidewire lumen.

23. The system of claim 21 wherein a distal end of the stabilizer member lumen is substantially axially coextensive with a distal end and work lumen port of the stabilizer sheath.

24. The system of claim 21 wherein the tissue penetration member is configured to penetrate tissue upon rotation.

25. The system of claim 24 wherein the tissue penetration member comprises a tubular needle with a sharpened distal end, a helical tissue penetration member disposed about the tubular needle and a torquable shaft having a distal end secured to a proximal portion of the tubular needle and a proximal portion of the helical tissue penetration member.

26. The system of claim 21 further comprising an ultrasound emission member and an ultrasound receiver disposed at the distal section of the stabilizer sheath.

27. A method of accessing the left atrium of a patient's heart from the right atrium of the patient's heart, comprising
providing a transmembrane access system, including:
a stabilizer sheath having an inner work lumen extending therein, having a port disposed on a distal end of the sheath and in communication with the inner lumen and having a stabilizer member lumen substantially parallel to a longitudinal axis of the stabilizer sheath disposed at the distal section,
an elongate stabilizer member that is configured to extend from the stabilizer member lumen and provide lateral support to the distal end of the stabilizer sheath,
a guide catheter having a shaped distal section that has a curved configuration in a relaxed state and an outer surface which is configured to move axially within a portion of the inner lumen of the stabilizer sheath that extends from the proximal end of the stabilizer sheath to the port; and
a tissue penetration member which is configured to move axially within an inner lumen of the guide catheter and which is axially extendable from a distal end of the guide catheter for membrane penetration;
advancing the stabilizer sheath through a superior vena cava of the patient and positioning the stabilizer sheath with the stabilizer member within the inferior vena cava and with the port of the stabilizer sheath adjacent the right atrium of the patient's heart;
advancing the distal end of the guide catheter through the inner work lumen of the stabilizer sheath until the distal end of the guide catheter is positioned adjacent a desired site of the septum of the patient's heart;
advancing the tissue penetration member from the distal end of the guide catheter; and
activating the tissue penetration actuator and advancing the tissue penetration member distally through the septum.

28. The method of claim 27 wherein the tissue penetration member is configured to penetrate tissue upon rotation and wherein the activation of the tissue penetration member comprises rotating the tissue penetration member.

29. The method of claim 27 wherein the system further comprising an ultrasound emission member and an ultrasound receiver disposed at the distal section of the stabilizer sheath and further comprising emitting ultrasound energy from the ultrasound emission member directed towards the desired site, receiving reflected ultrasound energy with the ultrasound energy receiver and generating information about the desired site from the reflected ultrasound energy.

30. A transmembrane access system, comprising:
a guide catheter having a shaped distal section that includes a curved configuration in a relaxed state, having an inner work lumen extending within a length thereof, having a port disposed on a distal end of the guide catheter and in communication with the inner work lumen and having a stabilizer member lumen which is substantially parallel to a nominal longitudinal axis of the guide catheter and which has a distal port disposed proximal of the shaped distal section;
an elongate stabilizer member that is configured to extend distally from the distal port of the stabilizer member lumen of the guide catheter and provide lateral support to the distal section of the guide catheter; and
a tissue penetration member which is configured to move axially within the inner work lumen of the guide catheter and which is axially extendable from a distal end of the guide catheter for membrane penetration.

31. The system of claim 30 wherein the tissue penetration member is configured to penetrate tissue upon rotation.

32. The system of claim 30 wherein the stabilizer member lumen extends proximally from the distal port thereof for a length less than about one half the overall length of the guide catheter.

33. The system of claim 32 wherein the length of the stabilizer member lumen is less than about 10 cm.

34. The system of claim 30 further comprising an ultrasound emission member and an ultrasound receiver disposed on a distal portion of the guide catheter.

35. The system of claim 34 wherein the ultrasound energy generator and ultrasound energy receiver comprise an ultrasound transducer.

36. The system of claim 30 further comprising an elongate dilator configured to slide axially within the working lumen of the guide catheter and having a distal stabilizer member lumen configured to allow axial passage of the elongate stabilizer member, the distal stabilizer member lumen including a proximal port and distal port configured to extend beyond a distal end of the guide catheter such that the distal stabilizer member lumen may extend distally beyond the distal end of the guide catheter.

37. The system of claim 36 wherein the proximal port of the distal stabilizer member lumen of the dilator opens to the side of the dilator and the distal port of the distal stabilizer member lumen opens in a distal direction from a distal tip of the elongate dilator.

38. A transmembrane access system, comprising
an elongate guide catheter having an inner work lumen and a distal port disposed in fluid communication with the inner work lumen, a shaped distal section that includes a curved configuration in a relaxed state, and a stabilizer member lumen which is substantially parallel to a longitudinal axis of the guide catheter, which has an intermediate port disposed proximal to the shaped distal section of the guide catheter and a distal port which is disposed in the shaped distal section of the guide catheter;
an elongate stabilizer member that is configured to extend from the intermediate port and distal port of the stabilizer member lumen and provide lateral support to a distal portion of the guide catheter; and
a tissue penetration member which is configured to move axially within the inner work lumen of the guide catheter and which is axially extendable from a distal end of the guide catheter for membrane penetration.

39. The system of claim 38 wherein the distal port of the stabilizer member lumen is axially coextensive with a distal end of the guide catheter.

40. The system of claim 38 wherein the stabilizer member lumen extends proximally from the intermediate port of the stabilizer member lumen for a length less than about one half the overall length of the guide catheter.

41. The system of claim 40 wherein the stabilizer member lumen extends proximally from the intermediate port a length less than about 10 cm.

42. A method of accessing the left atrium of a patient's heart from the right atrium of the patient's heart, comprising
providing a transmembrane access system, including:
a guide catheter having a shaped distal section that includes a curved configuration in a relaxed state, having an inner work lumen extending therein, having a port disposed on a distal end of the guide catheter and in communication with the inner work lumen and having a stabilizer member lumen substantially parallel to a nominal longitudinal axis of the guide catheter proximal of the shaped distal section;
an elongate stabilizer member that is configured to extend from a distal port of the stabilizer member lumen and provide lateral support to the distal end of the stabilizer sheath, and
a tissue penetration member which is configured to move axially within the inner work lumen of the guide catheter and which is axially extendable from the distal end of the guide catheter for membrane penetration;
advancing the guide catheter through a superior vena cava of the patient and positioning the guide catheter with the stabilizer member within the inferior vena cava and with the port of the guide catheter positioned adjacent a desired site of the septum of the patient's heart;
advancing the tissue penetration member from the port on the distal end of the guide catheter; and
activating the tissue penetration actuator and advancing the tissue penetration member distally through the septum.

43. The method of claim 42 wherein the system further comprises an ultrasound emission member and an ultrasound receiver disposed at the distal section of the guide catheter and further comprising emitting ultrasound energy from the ultrasound emission member directed towards a desired site, receiving reflected ultrasound energy with the ultrasound energy receiver and generating information about the desired site from the reflected ultrasound energy.
